(19) United States
(12) Patent Application Publication
(10) Pub. No.: US 2014/0128380 A1
(43) Pub. Date: May 8, 2014

(54) METHODS OF MODULATING THE ACTIVITY OF THE MC1 RECEPTOR AND TREATMENT OF CONDITIONS RELATED TO THIS RECEPTOR

(71) Applicant: Mimetica Pty Ltd, Milton (AU)

(72) Inventors: Mark Arnold Thomas Blaskovich, Bardon (AU); Peter Joseph Cassidy, Ashgrove (AU)

(73) Assignee: Mimetica Pty Ltd, Milton (AU)

(21) Appl. No.: 14/153,504

(22) Filed: Jan. 13, 2014

Related U.S. Application Data
(63) Continuation of application No. 13/203,674, filed on Feb. 21, 2012, now abandoned.

(51) Int. Cl. C07D 243/08; C07D 487/04; C07D 403/12; A61Q 19/04; C07D 405/12; C07D 413/12; C07D 417/12; A61Q 19/02; A61K 8/49; C07D 403/14

(52) U.S. Cl. CPC C07D 243/08; A61K 8/494; C07D 487/04; C07D 403/12; C07D 403/14; C07D 405/12; A61K 8/4973; C07D 413/12; C07D 417/12; A61Q 19/02; A61Q 19/04
USPC 514/221; 514/218; 435/375

(57) ABSTRACT

The present invention provides compounds of Formula (I) that are useful for binding and/or modulating the biological activity of the melanocortin-1 receptor (MC1R). Compounds of this invention can be used to treat diseases and/or conditions in which modulation of MC1R is beneficial. Such diseases and/or conditions include, but are not limited to, hyperpigmentation (including melasma), hypopigmentation (including vitiligo), melanoma, basal cell carcinoma, squamous cell carcinoma, erythropoietic protoporphyria, polymorphous light eruption, solar urticaria, photosensitivity, sunburn, inflammatory diseases, aberrant fibroblast activity and pain.

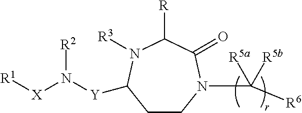

### CROSS-REFERENCE TO RELATED APPLICATIONS

This application is a continuation of U.S. application Ser. No. 13/203,674, filed on Feb. 21, 2012, which claims the benefit and priority to and is a U.S. National Phase Application of PCT International Application Number PCT/AU2009/000228, filed on Feb. 27, 2009, designating the United States of America and published in the English language. The disclosures of the above-referenced applications are hereby expressly incorporated by reference in their entireties.

### FIELD OF THE INVENTION

The present invention relates to methods of using compounds that bind to MC1R for modulation and binding of this receptor as well as in methods of treatment and diagnosis that utilise the binding activity of the compounds. The invention further relates to methods of modulating the activity of the melanocortin-1 receptor that rely on this binding activity of the compounds. In particular the present invention relates to the use of a family of 1,4-diazepan-2-ones and derivatives thereof to modulate the activity of the melanocortin-1 receptor. The invention also relates to methods and uses of the compounds in the diagnosis and treatment of conditions in which the activity or presence of melanocortin-1 receptor is implicated.

### BACKGROUND OF THE INVENTION

The melanocortin-1 receptor (MC1R) is a G-protein coupled receptor (GPCR) belonging to the family of melanocortin receptors. There are five melanocortin receptors that have been isolated and cloned to date: MC1R, MC2R, MC3R, MC4R and MC1R. The melanocortin receptors participate in a variety of physiologic functions, providing a number of opportunities for therapeutic intervention in physiologic processes through alteration (i.e., a statistically significant increase or decrease) or modulation (e.g., up-regulation or down-regulation) of melanocortin receptor signalling activity.

Reviews of the melanocortin receptors and their potential as therapeutic targets have been published (Wikberg 2000, Wikberg 2001, Voisey 2003, BOihm 2006). The melanocortin receptor family members are regulated by natural peptide agonists such as adrenocorticotropic hormone (ACTH) and the melanocyte-stimulating hormones (α-, β-, γ-MSH) derived from pro-opiomelanocortin (POMC), and by peptide antagonists such as Agouti signal protein (ASP) and Agouti-related peptide (AGRP). The MC1R is widely expressed and is associated with pigmentation in melanocytes and with inflammation responses in many cells involved in the immune system. The MC2R differs from the other melanocortin receptors in that it binds only ACTH but not MSH ligands. It is highly expressed in the adenal gland and controls corticosteroid synthesis. The MC3R is found in the brain, but also elsewhere in the body, and appears to play a role in the regulation of energy homeostasis, and possibly sexual dysfunction. The MC4R is found almost exclusively in the brain, with some reports of its presence elsewhere. It has been strongly associated with feeding control, and also implicated with sexual desire. The MC5R is widely expressed in peripheral tissues, particularly in the exocrine glands, with some receptor also expressed in the brain.

The MC1R was first cloned and expressed from humans and mice in 1992 (Chhajlani 2002, Mountjoy 2002). MC1R structure and functional regulation was reviewed in 2005 (García-Barrón 2005). The presence of human MC1R has been reported in a variety of cell lines and tissues, using a number of techniques (see summary in Roberts 2006). However, while analysis for MC1R mRNA in melanocytes and a variety of non-melanocytic cells using semiquantitative reverse transcriptase-polymerase chain reaction (RT-PCR) showed its presence in all cell types examined, quantitative real-time PCR revealed high levels only in melanocytic cells. Western immunoblotting revealed detectable MC1R protein in an MC1R-overexpressing HEK cell line and a melanoma cell line, but not in normal melanocytes or other cell lines. Endogenous MC1R protein in melanocytes could only be detected using ¹²⁵I-labeled NDP-MSH, which did not detect any surface protein in the other cell lines tested. Thus functionally significant levels of MC1R, particularly in the skin, may be restricted primarily to melanocytes (Roberts 2006, Roberts 2007).

The MC1R plays an essential role in regulating skin pigmentation (Slominski 2004, García-Barrón 2005, Böhm 2006, Lin 2007). Alpha-melanocyte stimulating hormone (α-MSH) signals via the MC1R in melanocytes to stimulate eumelanogenesis (the formation of the black pigment eumelanin) via upregulation of the enzyme tyrosinase and via melanocyte proliferation (Slominski 2004). Agouti protein and ASP (but not AGRP) antagonize this stimulation, shifting pigment production to the yellow pigment pheomelanin, while ACTH is another agonist. It has been shown in dogs that another peptide, β-defensin 103, binds to MC1R without agonism, but prevents inhibition by agouti protein and enables the production of eumelanin, resulting in black hair (Candille 2007). Mice with a loss of function MC1R gene mutation (Mc1r^{e}) are yellow, while mice with a constitutively active MC1R mutation (Mc1r^{som}) are black (Robbins 1993, Miller 1997). A number of studies have associated human MC1R gene polymorphs with diminished MC1R activity, resulting in fair skin color, red hair, and reduced tanning ability (Rees 2000, Naysmith 2004, Newton 2007, Pharoah 2008).

A more potent and stable analog of α-MSH, [Nle⁴-D-Phe⁷]-α-MSH (NDP-MSH, Melanotan I), causes a significant increase in eumelanin (but not pheomelanin) in human skin when dosed subcutaneously (Levine 1991, Dorr 2000, Dorr 2004, Barnetson 2006, Hadley 2006). This effect is also evident in humans with MC1R variant alleles (FitzGerald 2006). A tripeptide α-MSH antagonist causes depigmentation when injected or applied topically to the skin of the frog Xenopus laevis (Quillan 1995).

A variety of peptides (Holder 2002; Holder 2003, Bonetto 2005, Abdel-Malek 2006, Bednarek 2008), peptide derivatives (Mutulis 2005), peptidomimetics (Mazur 2003, Verdie 2007, Haskell-Luevano 1999) and small molecules (Mutulis 2007, Joseph 2008) that bind to and activate or inhibit the MC1R have been reported.

Pigmentary disorders are the third most common dermatologic disorder (Halder 2003) affecting patients and contribute to significant psychosocial impairment. The ability to alter skin pigmentation by activation or inhibition of MC1R has a variety of potential therapeutic applications. Agonists that activate MC1R and promote pigmentation hold the potential to reduce UV-induced skin damage and carcinogenesis (Brown 2001). These agonists might also be useful in the treatment of hypopigmentation disorders, such as vitiligo, certain forms of albinism, piebaldism, Waardenburg syndrome, Griscelli syndrome, and pigmentary mosaicism (Schaffer 2006). Vitiligo is the most common disorder leading to depigmented areas of the skin, resulting in white patches that usually increase in size with time. Existing treatment regimes consist of cosmetic camouflage, attempts at repigmentation (e.g topical steroids, topical vitamin D analogs, topical calcineurin inhibitors, UV treatment, topical psoralen or khellin with UV treatment, oral immunosuppressant treatments such as corticosteroids, cyclosporin, surgical skin grafts), or attempts at depigmentation of surrounding skin (e.g using a variety of topical agents, such as p-(benzyloxy)phenol, hydroquinones, phenols or mercuric iodide) (Gawkrodger 2008). These treatments are often ineffective and potentially harmful (Gawkrodger 2008, Olumide 2007). Reduced expression of MC1R (2.1 fold) in lesional skin, and increased expression of MC1R (1.6 fold) in non-lesional skin relative to healthy controls has been measured (Kingo 2007). An MC1R agonist that stimulated pigmentation in lesional skin or an MC1R antagonist that inhibited pigmentation in non-lesional skin could be a useful treatment for vitiligo.

In contrast, antagonists that inhibit MC1R and decrease pigmentation may prove useful for the treatment of hyperpigmentation disorders. Hyperpigmentation is a cosmetically important condition seen most often in middle-aged and elderly individuals as a result of exposure to ultraviolet light (melasma, solar lentigines, ephelides), certain drugs (eg, estrogens, tetracyclines, amiodarone, phenyloin, phenothiazines, sulfonamides) or chemicals (photosensitizing agents, bergamot oil, furocoumarins), or the existence of disease (erythromelanosis follicularis, linea fusca, poikiloderma of civatte, Riehl's melanosis, Addison's disease, hemochromatosis, liver disease, pituitary tumors) (Stulberg 2003a, Stulberg 2003b). Hyperpigmentation may also be a postinflammatory response to trauma, chemical peels, laser therapy, or acne. Treatment of hyperpigmentation can be frustrating because many agents cause skin irritation and require months of use before the results are apparent. Some are only partly effective. All require dedicated patient compliance with sunscreens to prevent reversal of the skin lightening effect. Skin-whitening, lightening or hypopigmentary agents such as those described for vitiligo treatment are often employed (Rendon 2005). An MC1R antagonist that inhibited pigmentation could be a useful treatment for these hyperpigmentation disorders.

Increasing skin pigmentation by activation of MC1R has a variety of potential therapeutic applications not directly related to pigmentation disorders. The photoprotective effect of increased pigmentation (“tanning”) is well known, and the ability to increase pigmentation without exposure to UV light provides a prophylactic treatment to reduce UV-related skin damage, especially that related to skin cancer, such as actinic keratosis, melanoma, basal cell carcinoma, and squamous cell carcinoma. MC1R gene polymorphisms are associated with an increased risk of melanoma (Stratigos 2006, Pharoah 2008, de-Misa 2008) and both basal and squamous cell carcinoma (Box 2001, Pharoah 2008). Activation of the tanning pathway by α-MSH shields DNA from UV damage via pigment formation capping cell nuclei, and also appears to initiate DNA repair and reduce hydrogen peroxide generation, providing a pigmentation-independent route for reduction of skin cancer (Wickelgren 2007, Abdel-Malek 2008). A tetrapeptide MC1R agonist protected human melanocyte cells from UV-induced DNA damage and cytotoxicity, an effect absent in melanocytes expressing inactive MC1R (Abdel-Malek 2006). Subcutaneous dosing with the MC1R agonist [Nle⁴-D-Phe⁷]-α-MSH in human volunteers led to reduced sunburn damage (reduction in apoptotic cells in the epidermis) and reduced DNA damage (reduction of thymine dimers in the epidermis) (Barnetson 2006).

Other photoprotective uses for an MC1R agonist include, but are no limited to, treatment in patients who are intolerant of sunlight, such as those with erythropoietic protoporphyria, polymorphous light eruption, solar urticaria, or those undergoing photodynamic therapy.

α-MSH shows immunosuppressive effects in humans, suppressing a variety of inflammation responses, and the MC1R has been implicated in these immunomodulating activities (Catania 2004). MC1R mRNA is expressed in inflammatory cell such as macrophages, lymphocytes, neutrophils, mast cells, dendritic cells, and mononuclear cells. Activation of MC1R in inflammatory cells by MC1R agonists reduced the inflammatory responses in cells treated with tumor necrosis factor α, such as inhibition of NF-κB-mediated transcription (Getting 2002, Catania 2004). An MC1R agonist might be expected to be useful as a treatment for both acute and chronic inflammatory reactions, such as allergic inflammation, autoimmunity, rheumatoid arthritis, inflammatory bowel disease, vasculitis, infections, septic shock, acute respiratory distress syndrome, hemorrhagic shock, ischemia and reperfusion injury, and organ transplantation (Catania 2004).

α-MSH appears to play a role in collagen regulation, with anti-fibrogenic activity. Human dermal fibroblasts express MC1R, providing possible therapeutic opportunities in skin disorders with aberrant fibroblast activity (Bohm 2006).

MC1R has also been associated with analgesia, with MC1R non functional gene variants (Mc1r^{e/e} mice and human red-heads) resulting in reduced sensitivity to painful stimuli and increased sensitivity to μ-opioid and κ-opioid analgesics (Mogil 2003, Mogil 2005). MC1R agonists or antagonists could be useful for moderating analgesic effects.

MC1R is over expressed in most murine and human melanoma metastases. (Siegrist 1989, Siegrist 1994). Various α-MSH peptide derivatives that can recognize the MC1R in vitro or in vivo have been radiolabeled (such as with ¹⁸F (Vaidyanathan 1997), ⁹⁹ mTc (Chen 1999, Chen 2000), ¹¹¹In (Cheng 2002, Chen 2001, Froidevaux 2002, Froidevaux 2005, Bagutti 1994, Bard 1995), ¹²⁵I (Cheng 2004), ⁶⁷Ga (Froidevaux 2004), ⁸⁶Y (McQuade 2005), or ⁶⁴Cu (McQuade 2005, Cheng 2005, Cheng 2007)) and used to detect melanoma cells and malignant growths. Moreover, an α-MSH peptide, ReCCMSH (Arg¹¹) radiolabeled with a therapeutic radionuclide (either ¹⁸⁸Re or ²¹²Pb) has provided initial experimental evidence of efficacy for the treatment of tumours in mice bearing either B16F1 murine or TXM13 human xenografted melanoma (Miao 2005a, Miao 2005b). These results highlight the potential of using molecules that target MC1R which can be labelled with a detectable label for use in diagnostic or monitoring applications, or which may be used as molecular targeting agents to deliver active agents such as radionuclides to the receptor for use as targeted therapeutics.

For the reasons described above it would be desirable to provide molecules that bind to and/or modulate MC1R for potential use in a number of therapeutic areas. Therapeutic regulation of biological signal transduction includes modulation of MC1R-mediated cellular events including, inter alia, inhibition or potentiation of interactions among MC1R-binding and activating or deactivating molecules, or of other agents that regulate MC1R activities. An increased ability to so bind and/or regulate MC1R may facilitate the development of methods for modulating melanin production or other biological processes, and for treating conditions associated with such pathways such as hyperpigmentation, hypopigmentation photosensitivity, melanoma, carcinoma, inflammation and analgesia as described above.

Accordingly there is still the need to develop improved methods of binding to and/or modulating the activity of MC1R which would facilitate the diagnosis, monitoring and treatment of MC1R related conditions.

### SUMMARY OF THE INVENTION

The present invention provides a method of modulating the activity of MC1R or a fragment, analogue or functional equivalent thereof comprising exposing the MC1R or a fragment or analogue or functional equivalent thereof to a compound of the formula (I):

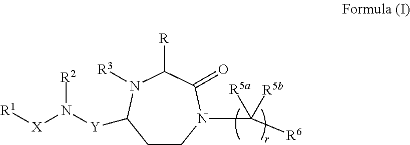

wherein

Y is a group of formula —(CR⁹R¹⁰)ₙ—;

X is selected from the group consisting of —C(═O)—, —OC(═O)—, —NHC(═O)—, —(CR¹¹R¹²)ₛ, and —S(═O)₂—;

R is an amino acid side chain group;

R¹ is selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂heterocycloalkyl, optionally substituted C₆-Cisaryl, and optionally substituted C₁-C₁₈heteroaryl;

R² and R³ are each independently selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂heterocycloalkyl, optionally substituted C₆-C₁₈aryl, and optionally substituted C₁-C₁₈heteroaryl, or

R² and R³ may be joined to form a linker between the two nitrogen atoms to which they are attached, wherein the linker is selected from the group consisting of —C(═O)—, —CH₂—, —C(═O)CH₂ and —CH₂C(═O)—;

R^{5a}, R^{5b} and R⁶ are each independently selected from the group consisting of H, halogen, hydroxy, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₁-C₁₀heteroalkenyl, optionally substituted C₃-C₁₂cycloalkyl, optionally substituted C₂-C₁₂heterocycloalkyl, optionally substituted C₆-C₁₈aryl, optionally substituted C₁-C₁₈heteroaryl, optionally substituted amino, optionally substituted carboxy, optionally substituted carboxamide, optionally substituted C₁-C₁₂alkyloxy, and optionally substituted thio;

each R⁹ and R¹⁰ is independently selected from the group consisting of H, optionally substituted C₁-C₁₂alkyl, optionally substituted C₆-C₁₈aryl, and optionally substituted C₁-C₁₈heteroaryl;

each R¹¹ and R¹² is independently selected from the group consisting of H, and optionally substituted C₁-C₁₂alkyl;

n is an integer selected from the group consisting of 1, 2, 3 and 4;

r is an integer selected from the group consisting of 0, 1, 2, 3, and 4;

s is an integer selected from the group consisting of 0, 1, 2, 3, and 4;

or a pharmaceutically acceptable salt or prodrug thereof.

In one embodiment the MC1R or fragment or analogue or functional equivalent thereof is in a cell and the method comprises exposing the cell to a compound of formula (I). In one embodiment the invention provides a method of modulating the activity of MC1R or fragment or analogue or functional equivalent thereof in a mammal comprising administering a MC1R-modulating amount of a compound of formula (I) to the mammal.

In yet a further aspect the invention provides the use of a compound of the formula (I) in modulating the activity of MC1R or a fragment, analogue or functional equivalent thereof.

In yet a further aspect the invention provides the use of a compound of formula (I) in the preparation of a medicament for modulating the activity of MC1R or fragment or analogue or functional equivalent thereof in a mammal.

In yet an even further aspect the invention provides a method of binding a compound of formula (I) or labelled form thereof to MC1R or a fragment, analogue or functional equivalent thereof, the method comprising exposing the MC1R or a fragment, analogue or functional equivalent thereof to a compound of formula (I) or a labelled form thereof. In the method of binding of the invention the compounds of formula (I) may inherently contain a label such as where they contain an internal label such as a radioisotope of one or more of the atoms contained in the compound. The exact isotope chosen will depend upon the mode of detection desired and will be chosen by a skilled addressee in the art. Alternatively the compounds of formula (I) may be labelled by addition of a separate label (such as a fluorescent label or the like to the compound of formula (I)). The incorporation of labels of this type is well known in the art and a skilled addressee would be readily able to determine a suitable label depending upon the desired use of the label.

In general the MC1R or a fragment, analogue or functional equivalent thereof is labelled for diagnostic or monitoring purposes and the method further comprises detecting the presence of the compound of formula (I) or labelled form thereof. The mode of detection will depend upon the exact form of label chosen and the type of label will determine the means of detection used.

The ability of the compounds of formula (I) to bind to MC1R or a fragment, analogue or derivative thereof may be used to deliver one or more active agents to the receptor. Accordingly in a further aspect the invention provides a method of delivering an active agent to MC1R or a fragment, analogue or functional equivalent thereof in a mammal, the method comprising administering a compound of formula (I) as described in claim 1 substituted with or attached to an active agent to the mammal. The binding of the compound to the receptor therefore effectively delivers the active agent to the receptor and this can be used in therapeutic applications such as chemotherapy.

In yet an even further aspect the invention provides a composition for inducing UV-independent pigmentation of human skin and/or for enhancing UV-dependent pigmentation of human skin, comprising a compound of formula (I) and a dermatologically acceptable carrier, excipient or diluent, wherein the composition is formulated to penetrate the human skin to the stratum basale.

In one embodiment the composition further comprises at least one UVA-stabilizing and/or UVB-stabilizing screening agent. In another embodiment the composition comprises at least one photo-protective agent. In another embodiment the composition comprises at least one compound selected from the group consisting of: physical sunblocks, sunscreens and free-radical scavengers. In yet an even further embodiment the composition further comprises at least one compound selected from the group consisting of: an anti-inflammatory agents, an anti-acne agents, anti-wrinkle agent, an anti-scarring agent, an anti-psoriatic agents, an anti-proliferative agent, an antifungal agent, an anti-viral agent, an anti-septic agent, a local anaesthetic, a keratolytic agents, a hair growth stimulant, and a hair growth inhibitor.

In yet an even further aspect the invention provides a composition for reducing pigmentation of human skin, comprising a compound of formula (I) and a dermatologically acceptable carrier, excipient or diluent, wherein the composition is formulated to penetrate the human skin to the stratum basale.

In another embodiment the composition comprises at least one photo-protective agent. In another embodiment the composition comprises at least one compound selected from the group consisting of: physical sunblocks, sunscreens and free-radical scavengers.

In another aspect the invention provides a composition for inducing UV-independent pigmentation of human skin and/or for enhancing UV-dependent pigmentation of human skin, comprising a compound of formula (I), formulated to penetrate the human skin to the stratum basale, and provided in an amount sufficient to cause macroscopically observable pigmentation when applied to human skin.

In another aspect the invention provides a dermatological or cosmetological composition for an external topical administration to human skin, comprising together with pharmaceutically and/or cosmetologically acceptable excipients: at least one UVA-stabilizing and/or UVB-stabilizing screening agent, and a compound of formula (I), formulated to penetrate the human skin to the stratum basale, and provided in an amount to cause macroscopically observable pigmentation when applied to human skin.

In another aspect the invention provides a composition for inducing UV-independent pigmentation of human skin, comprising a compound of formula (I), formulated for oral administration, which acts systemically on melanocytes in the skin to induce melanogenesis, and provided in an amount to cause macroscopically observable pigmentation.

In certain embodiments, the subject compositions are provided in the form of a gel, a cream or a lotion. In certain embodiments, the composition is less irritating when applied to skin than a compound of formula (I) applied to skin alone.

In another aspect the invention provides a method for inducing UV-independent pigmentation of human skin, comprising of administering any of the subject compositions in an amount to cause macroscopically observable pigmentation when applied to human skin.

In another aspect the invention provides a method for protecting human skin from ultraviolet radiation, comprising of administering any of the subject compositions in an amount to cause macroscopically observable pigmentation when applied to human skin.

In another aspect the invention provides a method for reducing the rate of formation of solar erythema, solar allergies or solar elastosis, comprising of administering any of the subject compositions in an amount to cause macroscopically observable pigmentation when applied to human skin.

In another aspect the invention provides a method for preventing or delaying actinic ageing of human skin, comprising of administering any of the subject compositions in an amount to cause macroscopically observable pigmentation when applied to human skin.

In another aspect the invention provides a method for treating or preventing a disease or disorder in a mammal caused by ultraviolet radiation, comprising of administering any of the subject compositions in an amount to cause macroscopically observable pigmentation when applied to human skin.

In another aspect of the invention provides a composition for reducing pigmentation of human skin, comprising a compound of formula (I), formulated to penetrate the human skin to the stratum basale, and provided in an amount sufficient to reduce pigmentation when applied to human skin.

In another aspect the invention provides a dermatological or cosmetological composition for an external topical administration to human skin, comprising together with pharmaceutically and/or cosmetologically acceptable excipients: at least one UVA-stabilizing and/or UVB-stabilizing screening agent, and a compound of formula (I), formulated to penetrate the human skin to the stratum basale, and provided in an amount to reduce pigmentation when applied to human skin.

In another aspect the invention provides a composition for reducing pigmentation of human skin, comprising a compound of formula (I), formulated for oral administration, which acts systemically on melanocytes in the skin to reduce melanogenesis, and provided in an amount sufficient to reduce pigmentation when delivered orally.

In yet an even further aspect the invention provides a method of preventing or treating a condition in a mammal wherein the condition is selected from the group consisting of (i) conditions associated with the activity or presence of MC1R or a fragment, analogue or functional equivalent thereof in a mammal and (ii) conditions that may be prevented or treated by modification of skin pigmentation in the mammal, the method comprising administering a therapeutically effective amount of a compound of formula (I) as described above to the mammal.

In yet a further aspect the invention provides a method of modifying the level of pigmentation in the skin of a mammal, the method comprising administering a MC1R-modulating amount of a compound of formula (I) as described above to the mammal.

The compound may be administered in any way known in the art although in one aspect the compound is administered topically. In another aspect the compound is administered orally. In another aspect the compound is administered parenterally.

In one embodiment of the methods and uses of the invention the activity of MC1R is up-regulated. In one embodiment the activity of MC1R or a fragment, analogue or functional equivalent thereof is up regulated in a mammal leading to an increase in pigmentation of the skin of the mammal.

In one embodiment of the methods and uses of the invention the activity of MC1R is down-regulated. In one embodiment the activity of MC1R or a fragment, analogue or functional equivalent thereof is down regulated in the mammal leading to a decrease in pigmentation of the skin of the mammal.

In one embodiment of the invention the condition is a condition that may be prevented or treated by modification of skin pigmentation in the mammal. In one embodiment the condition is selected from the group consisting of skin damage caused by UV radiation, solar erythema, solar allergies, solar elastosis, actinic ageing of the skin and disorders associated with ultraviolet radiation.

In one embodiment of the method the condition is selected from the group consisting of hyperpigmentation (including melasma), hypopigmentation (including vitiligo), melanoma, basal cell carcinoma, squamous cell carcinoma, erythropoietic protoporphyria, polymorphous light eruption, solar urticaria, photosensitivity, sunburn, inflammatory diseases, aberrant fibroblast activity and pain. In one embodiment the compound of formula (I) is administered in combination with a second active agent.

In some embodiments of the methods involving administration of the compound of formula (I) the compound is administered in the form of a composition, the composition comprising a compound of formula (I) and a dermatologically acceptable carrier, excipient or diluent, wherein the composition is formulated to penetrate the human skin to the stratum basale.

In some embodiments the composition comprises at least one UVA-stabilizing and/or UVB-stabilizing screening agent. In some embodiments the composition comprises at least one photo-protective agent. In some embodiments the composition comprises at least one agent selected from the group consisting of: physical sunblock agents, sunscreen agents and free-radical scavenging agents. In some embopdiments the composition further comprises at least one agent selected from the group consisting of: an anti-inflammatory agents, an anti-acne agents, anti-wrinkle agent, an anti-scarring agent, an anti-psoriatic agents, an anti-proliferative agent, an antifungal agent, an anti-viral agent, an anti-septic agent, a local anaesthetic, a keratolytic agents, a hair growth stimulant, and a hair growth inhibitor.

In another aspect the invention provides for the use of a compound of formula (I) in the preparation of a medicament for treating a condition in a mammal selected from the group consisting of (i) conditions associated with the activity or presence of MC1R or a fragment, analogue or functional equivalent thereof in the mammal and (ii) conditions that may be prevented or treated by modification of skin pigmentation in the mammal.

In another aspect the invention provides the use of a compound of formula (I) as described above in the preparation of a medicament for modifying the level of pigmentation in the skin of a mammal.

In one aspect the medicament is adapted to be administered topically. In another aspect the medicament is adapted to be administered orally. In another aspect the medicament is adapted to be administered parenterally.

In one embodiment of the use the condition is selected from the group consisting of hyperpigmentation (including melasma), hypopigmentation (including vitiligo), melanoma, basal cell carcinoma, squamous cell carcinoma, erythropoietic protoporphyria, polymorphous light eruption, solar urticaria, photosensitivity, sunburn, inflammatory diseases, aberrant fibroblast activity and pain. In one embodiment the medicament contains a second active agent.

In one embodiment the medicament is formulated to penetrate the human skin to the stratum basale. In one embodiment the medicament comprises at least one UVA-stabilizing and/or UVB-stabilizing screening agent. In one embodiment the medicament comprises at least one photo-protective agent. In one embodiment the medicament comprises at least one agent selected from the group consisting of: physical sunblock agents, sunscreen agents and free-radical scavenging agents.

In one embodiment the medicament further comprises at least one agent selected from the group consisting of: an anti-inflammatory agents, an anti-acne agents, anti-wrinkle agent, an anti-scarring agent, an anti-psoriatic agents, an anti-proliferative agent, an antifungal agent, an anti-viral agent, an anti-septic agent, a local anaesthetic, a keratolytic agents, a hair growth stimulant, and a hair growth inhibitor.

### DETAILED DESCRIPTION OF THE INVENTION

In this specification a number of terms are used which are well known to a skilled addressee. Nevertheless for the purposes of clarity a number of terms will be defined.

As used herein, the term “unsubstituted” means that there is no substituent or that the only substituents are hydrogen.

The term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R^{b}, C(═NOH)R^{a}, C(═NRa)NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NRb)NR^{c}R^{d}, NRaSO₂R^{b}, —SR^{a}, SO₂NR^{a}R^{b}, —OR^{a}, OC(═O)NR^{a}R^{b}, OC(═O)Ra and acyl,

wherein R^{a}, R^{b}, R^{c} and R^{d} are each independently selected from the group consisting of H, C₁-C₁₂ alkyl, C₁-C₁₂ haloalkyl, C₂-C₁₂ alkenyl, C₂-C₁₂ alkynyl, C₁-C₁₀ heteroalkyl, C₃-C₁₂ cycloalkyl, C₃-C₁₂ cycloalkenyl, C₁-C₁₂ heterocycloalkyl, C₁-C₁₂ heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In one embodiment each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, and CN.

The term “amino acid side chain group” represents a natural or unnatural side chain group present in a protein. The term includes side chain moieties present in naturally occurring proteins including the naturally occurring amino acid side chain moieties identified in table 1 below.

TABLE 1
Amino Acid Side Chain Moieties

| Amino Acid Side Chain Moiety | Amino Acid |
|---|---|
| H | Glycine |
| CH₃ | Alanine |
| CH(CH₃)₂ | Valine |
| CH₂CH(CH₃)₂ | Leucine |
| CH(CH₃)CH₂CH₃ | Isoleucine |
| (CH₂)₄NH₃⁺ | Lysine |
| (CH₂)₃NHC(NH₂)NH₂₊ | Arginine |
| CH₂-(imidazol-4-yl) | Histidine |
| CH₂COO⁻ | Aspartic Acid |
| CH₂CH₂COO⁻ | Glutamic acid |
| CH₂CONH₂ | Asparagine |
| CH₂CH₂CONH₂ | Glutamine |
| CH₂Ph | Phenylalanine |
| CH₂C₆H₄OH | Tyrosine |
| CH₂(Indolin-3-yl) | Tryptophan |
| CH₂SH | Cysteine |
| CH₂CH₂SCH₃ | Methionine |
| CH₂OH | Serine |
| CH(OH)CH₃ | Threonine |

In addition to naturally occurring amino acid side chain groups as identified above the term also includes derivatives or analogs thereof. As used herein the term derivative or analogue of an amino acid side chain group includes modifications and variations to naturally occurring side chain groups. With reference to the table above most of the naturally occurring amino acid side chain groups may be classified as alkyl, aryl, arylalkyl or heteroalkyl moieties. As such derivatives of amino acid side chain groups include straight or branched, cyclic or non-cyclic alkyl, aryl, heteroaryl, heteroarylalkyl, arylalkyl or heteroalkyl moieties.

Amino acid side chain groups as discussed above also include optionally substituted derivatives of alkyl, aryl, arylalkyl, heteroaryl, heteroarylalkyl, or heteroalkyl moieties. The optional substituents may be selected from the group defined above. For example, the optional substituents may be selected from but are not limited to OH, Cl, Br, F, COOH, COOR^{Z}, CONH₂, NH₂, NHR^{Z}, NR^{Z}R^{Z}, SH, SR^{Z}, SO₂R^{Z}, SO₂H and SOR^{Z} wherein R^{Z} is an alkyl, aryl or arylalkyl moiety.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

Several terms are prefaced by a modifier indicating the number of carbon atoms present in the moiety. For example, the modifier “C₁-C₆” in front of the term “alkyl” indicates that the alkyl moiety has from 1 to 6 carbon atoms. Further, the modifier “C₁-C₁₈” in front of the term “heteroaryl” indicates that the heteroaromatic ring may have from 1 to 18 carbon atoms as part of the total number of atoms in the ring system.

“Active agent” means a material or compound that has activity against the desired target. For example, in relation to a medical condition an active agent is one which when administered to a subject having the condition leads to a therapeutically benefical result in the subject.

“Acyl” means an R—C(═O)— group in which the R group may be an alkyl, cycloalkyl, heterocycloalkyl, aryl or heteroaryl group as defined herein. Examples of acyl include acetyl and benzoyl. The group may be a terminal group or a bridging group. If the group is a terminal group it is bonded to the remainder of the molecule through the carbonyl carbon.

“Acylamino” means an R—C(═O)—NH— group in which the R group may be an alkyl, cycloalkyl, heterocycloalkyl, aryl or heteroaryl group as defined herein. The group may be a terminal group or a bridging group. If the group is a terminal group it is bonded to the remainder of the molecule through the nitrogen atom.

“Alkenyl” as a group or part of a group denotes an aliphatic hydrocarbon group containing at least one carbon-carbon double bond and which may be straight or branched preferably having 2-14 carbon atoms, more preferably 2-12 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkenyloxy” refers to an alkenyl-O— group in which alkenyl is as defined herein. Preferred alkenyloxy groups are C₁-C₆ alkenyloxy groups. The group may be a terminal group or a bridging group. If the group is a terminal group it is bonded to the remainder of the molecule through the oxygen atom.

“Alkyl” as a group or part of a group refers to a straight or branched aliphatic hydrocarbon group, preferably a C₁-C₁₄ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a Alkyl-NH— group, in which alkyl is as defined herein.

“Dialkylamino” means a (alkyl)₂N— group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkylaminocarbonyl” refers to a group of the formula (Alkyl)ₓ(H)_{y}NC(═O)— in which x is 1 or 2, and the sum of x+y=2. The group may be a terminal group or a bridging group. If the group is a terminal group it is bonded to the remainder of the molecule through the carbonyl carbon.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group.

“Alkyloxyalkyl” refers to an alkyloxy-alkyl-group in which the alkyloxy and alkyl moieties are as defined herein. The group may be a terminal group or a bridging group. If the group is a terminal group it is bonded to the remainder of the molecule through the alkyl group.

“Alkyloxyary” refers to an alkyloxy-aryl-group in which the alkyloxy and aryl moieties are as defined herein. The group may be a terminal group or a bridging group. If the group is a terminal group it is bonded to the remainder of the molecule through the aryl group.

“Alkyloxycarbonyl” refers to an alkyl-O—C(═O)— group in which alkyl is as defined herein. The alkyl group is preferably a C₁-C₆ alkyl group. Examples include, but are not limited to, methoxycarbonyl and ethoxycarbonyl. The group may be a terminal group or a bridging group. If the group is a terminal group it is bonded to the remainder of the molecule through the carbonyl carbon.

“Alkyloxycycloalkyl” refers to an alkyloxy-cycloalkyl-group in which the alkyloxy and cycloalkyl moieties are as defined herein. The group may be a terminal group or a bridging group. If the group is a terminal group it is bonded to the remainder of the molecule through the cycloalkyl group.

“Alkyloxyheteroary” refers to an alkyloxy-heteroaryl-group in which the alkyloxy and heteroaryl moieties are as defined herein. The group may be a terminal group or a bridging group. If the group is a terminal group it is bonded to the remainder of the molecule through the heteroaryl group.

“Alkyloxyheterocycloalkyl” refers to an alkyloxy-heterocycloalkyl-group in which the alkyloxy and heterocycloalkyl moieties are as defined herein. The group may be a terminal group or a bridging group. If the group is a terminal group it is bonded to the remainder of the molecule through the heterocycloalkyl group.

“Alkylsulfinyl” means an alkyl-S-(═O)— group in which alkyl is as defined herein. The alkyl group is preferably a C₁-C₆ alkyl group. Exemplary alkylsulfinyl groups include, but not limited to, methylsulfinyl and ethylsulfinyl. The group may be a terminal group or a bridging group. If the group is a terminal group it is bonded to the remainder of the molecule through the sulfur atom.

“Alkylsulfonyl” refers to an alkyl-S(═O)₂— group in which alkyl is as defined above. The alkyl group is preferably a C₁-C₆ alkyl group. Examples include, but not limited to methylsulfonyl and ethylsulfonyl. The group may be a terminal group or a bridging group. If the group is a terminal group it is bonded to the remainder of the molecule through the sulfur atom.

“Alkynyl” as a group or part of a group means an aliphatic hydrocarbon group containing a carbon-carbon triple bond and which may be straight or branched preferably having from 2-14 carbon atoms, more preferably 2-12 carbon atoms, more preferably 2-6 carbon atoms in the normal chain. Exemplary structures include, but are not limited to, ethynyl and propynyl. The group may be a terminal group or a bridging group.

“Alkynyloxy” refers to an alkynyl-O— group in which alkynyl is as defined herein. Preferred alkynyloxy groups are C₁-C₆ alkynyloxy groups. The group may be a terminal group or a bridging group. If the group is a terminal group it is bonded to the remainder of the molecule through the oxygen atom.

“Aminoalkyl” means an NH₂-alkyl-group in which the alkyl group is as defined herein. The group may be a terminal group or a bridging group. If the group is a terminal group it is bonded to the remainder of the molecule through the alkyl group.

“Aminosulfonyl” means an NH₂—S(═O)₂— group. The group may be a terminal group or a bridging group. If the group is a terminal group it is bonded to the remainder of the molecule through the sulfur atom.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group. Typically an aryl group is a C₆-C₁₈ aryl group.

“Arylalkenyl” means an aryl-alkenyl-group in which the aryl and alkenyl are as defined herein. Exemplary arylalkenyl groups include phenylallyl. The group may be a terminal group or a bridging group. If the group is a terminal group it is bonded to the remainder of the molecule through the alkenyl group.

“Arylalkyl” means an aryl-alkyl-group in which the aryl and alkyl moieties are as defined herein. Preferred arylalkyl groups contain a C₁₋₅alkyl moiety. Exemplary arylalkyl groups include benzyl, phenethyl, 1-naphthalenemethyl and 2-naphthalenemethyl. The group may be a terminal group or a bridging group. If the group is a terminal group it is bonded to the remainder of the molecule through the alkyl group.

“Arylalkyloxy” refers to an aryl-alkyl-O— group in which the alkyl and aryl are as defined herein. The group may be a terminal group or a bridging group. If the group is a terminal group it is bonded to the remainder of the molecule through the oxygen atom.

“Arylamino” includes both mono-arylamino and di-arylamino unless specified. Mono-arylamino means a group of formula arylNH—, in which aryl is as defined herein. di-arylamino means a group of formula (aryl)₂N— where each aryl may be the same or different and are each as defined herein for aryl. The group may be a terminal group or a bridging group. If the group is a terminal group it is bonded to the remainder of the molecule through the nitrogen atom.

“Arylheteroalkyl” means an aryl-heteroalkyl-group in which the aryl and heteroalkyl moieties are as defined herein. The group may be a terminal group or a bridging group. If the group is a terminal group it is bonded to the remainder of the molecule through the heteroalkyl group.

“Aryloxy” refers to an aryl-O— group in which the aryl is as defined herein. Preferably the aryloxy is a C₆-C₁₈aryloxy, more preferably a C₆-C₁₀aryloxy. The group may be a terminal group or a bridging group. If the group is a terminal group it is bonded to the remainder of the molecule through the oxygen atom.

“Arylsulfonyl” means an aryl-S(═OO)₂— group in which the aryl group is as defined herein. The group may be a terminal group or a bridging group. If the group is a terminal group it is bonded to the remainder of the molecule through the sulfur atom.

A “bond” is a linkage between atoms in a compound or molecule. The bond may be a single bond, a double bond, or a triple bond.

“Carboxamide” refers to a group of the formula —C(═O)—NR₂ wherein each R is independently H, alkyl, alkenyl, alkynyl, aryl or heteroaryl as defined herein.

“Cyclic group” refers to saturated, partially unsaturated or fully unsaturated monocyclic, bicyclic or polycyclic ring system. Examples of cyclic groups include cycloalkyl, cycloalkenyl and aryl.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

“Cycloalkyl” refers to a saturated monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

“Cycloalkylalkyl” means a cycloalkyl-alkyl-group in which the cycloalkyl and alkyl moieties are as defined herein. Exemplary monocycloalkylalkyl groups include cyclopropylmethyl, cyclopentylmethyl, cyclohexylmethyl and cycloheptylmethyl. The group may be a terminal group or a bridging group. If the group is a terminal group it is bonded to the remainder of the molecule through the alkyl group.

“Cycloalkylalkenyl” means a cycloalkyl-alkenyl-group in which the cycloalkyl and alkenyl moieties are as defined herein. The group may be a terminal group or a bridging group. If the group is a terminal group it is bonded to the remainder of the molecule through the alkenyl group.

“Cycloalkylheteroalkyl” means a cycloalkyl-heteroalkyl-group in which the cycloalkyl and heteroalkyl moieties are as defined herein. The group may be a terminal group or a bridging group. If the group is a terminal group it is bonded to the remainder of the molecule through the heteroalkyl group.

“Cycloalkyloxy” refers to a cycloalkyl-O— group in which cycloalkyl is as defined herein. Preferably the cycloalkyloxy is a C₁-C₆cycloalkyloxy. Examples include, but are not limited to, cyclopropanoxy and cyclobutanoxy. The group may be a terminal group or a bridging group. If the group is a terminal group it is bonded to the remainder of the molecule through the oxygen atom.

“Cycloalkenyloxy” refers to a cycloalkenyl-O— group in which the cycloalkenyl is as defined herein. Preferably the cycloalkenyloxy is a C₁-C₆cycloalkenyloxy. The group may be a terminal group or a bridging group. If the group is a terminal group it is bonded to the remainder of the molecule through the oxygen atom.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 10, more preferably from 1 to 6, most preferably 1 to 3. m is typically 1 to 6, more preferably 1 to 3. Examples of haloalkyl include fluoromethyl, difluoromethyl and trifluoromethyl.

“Haloalkenyl” refers to an alkenyl group as defined herein in which one or more of the hydrogen atoms has been replaced with a halogen atom independently selected from the group consisting of F, Cl, Br and I.

“Haloalkynyl” refers to an alkynyl group as defined herein in which one or more of the hydrogen atoms has been replaced with a halogen atom independently selected from the group consisting of F, Cl, Br and I.

“Halogen” represents chlorine, fluorine, bromine or iodine.

“Heteroalkyl” refers to a straight- or branched-chain alkyl group preferably having from 2 to 14 carbons, more preferably 2 to 10 carbons in the chain, one or more of which has been replaced by a heteroatom selected from S, O, P and N. Exemplary heteroalkyls include alkyl ethers, secondary and tertiary alkyl amines, amides, alkyl sulfides, and the like. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

“Heteroarylalkyl” means a heteroaryl-alkyl group in which the heteroaryl and alkyl moieties are as defined herein. Preferred heteroarylalkyl groups contain a lower alkyl moiety. Exemplary heteroarylalkyl groups include pyridylmethyl. The group may be a terminal group or a bridging group. If the group is a terminal group it is bonded to the remainder of the molecule through the alkyl group.

“Heteroarylalkenyl” means a heteroaryl-alkenyl-group in which the heteroaryl and alkenyl moieties are as defined herein. The group may be a terminal group or a bridging group. If the group is a terminal group it is bonded to the remainder of the molecule through the alkenyl group.

“Heteroarylheteroalkyl” means a heteroaryl-heteroalkyl-group in which the heteroaryl and heteroalkyl moieties are as defined herein. The group may be a terminal group or a bridging group. If the group is a terminal group it is bonded to the remainder of the molecule through the heteroalkyl group.

“Heteroaryloxy” refers to a heteroaryl-O— group in which the heteroaryl is as defined herein. Preferably the heteroaryloxy is a C₁-C₁₂heteroaryloxy. The group may be a terminal group or a bridging group. If the group is a terminal group it is bonded to the remainder of the molecule through the oxygen atom.

“Heterocyclic” refers to saturated, partially unsaturated or fully unsaturated monocyclic, bicyclic or polycyclic ring system containing at least one heteroatom selected from the group consisting of nitrogen, sulfur and oxygen as a ring atom. Examples of heterocyclic moieties include heterocycloalkyl, heterocycloalkenyl and heteroaryl.

“Heterocycloalkenyl” refers to a heterocycloalkyl as defined herein but containing at least one double bond. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. The group may be a terminal group or a bridging group.

“Heterocycloalkylalkyl” refers to a heterocycloalkyl-alkyl-group in which the heterocycloalkyl and alkyl moieties are as defined herein. Exemplary heterocycloalkylalkyl groups include (2-tetrahydrofuryl)methyl, (2-tetrahydrothiofuranyl)methyl. The group may be a terminal group or a bridging group. If the group is a terminal group it is bonded to the remainder of the molecule through the alkyl group.

“Heterocycloalkylalkenyl” refers to a heterocycloalkyl-alkenyl-group in which the heterocycloalkyl and alkenyl moieties are as defined herein. The group may be a terminal group or a bridging group. If the group is a terminal group it is bonded to the remainder of the molecule through the alkenyl group.

“Heterocycloalkylheteroalkyl” means a heterocycloalkyl-heteroalkyl-group in which the heterocycloalkyl and heteroalkyl moieties are as defined herein. The group may be a terminal group or a bridging group. If the group is a terminal group it is bonded to the remainder of the molecule through the heteroalkyl group.

“Heterocycloalkyloxy” refers to a heterocycloalkyl-O— group in which the heterocycloalkyl is as defined herein. Preferably the heterocycloalkyloxy is a C₁-C₆heterocycloalkyloxy. The group may be a terminal group or a bridging group. If the group is a terminal group it is bonded to the remainder of the molecule through the oxygen atom.

“Heterocycloalkenyloxy” refers to a heterocycloalkenyl-O— group in which heterocycloalkenyl is as defined herein. Preferably the heterocycloalkenyloxy is a C₁-C₆ heterocycloalkenyloxy. The group may be a terminal group or a bridging group. If the group is a terminal group it is bonded to the remainder of the molecule through the oxygen atom.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH)ₓ. In groups of this type n is typically from 1 to 10, more preferably from 1 to 6, most preferably 1 to 3. x is typically 1 to 6, more preferably 1 to 3.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl). The group may be a terminal group or a bridging group.

“Sulfinyl” means an R—S(═O)— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfinylamino” means an R—S(═O)—NH— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the nitrogen atom.

“Sulfonyl” means an R—S(═O)₂— group in which the R group may be OH, alkyl, cycloalkyl, heterocycloalkyl; aryl or heteroaryl group as defined herein. The group may be a terminal group or a bridging group. If the group is a terminal group it is bonded to the remainder of the molecule through the sulfur atom.

“Sulfonylamino” means an R—S(═O)₂—NH— group. The group may be a terminal group or a bridging group. If the group is a terminal group it is bonded to the remainder of the molecule through the nitrogen atom.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

The present invention includes all pharmaceutically acceptable isotopically-labeled compounds of formula (I) wherein one or more atoms have the same atomic number as, but an atomic mass or mass number different from, the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

A number of the examples discussed above are indications of the ways in which the compounds of formula (I) may be internally labelled for use in the methods of binding of the present invention. As also discussed above the compounds of formula (I) may also be labelled by addition of a separate and distinct label to the molecule by way of a covalent bond. The additional label may be a fluorescent label or a radioactive label. Suitable labels to be added to compounds for these types of purposes are well known in the art. As used herein the term “label” includes any moiety or item detectable by spectroscopic, photochemical, biochemical, immunochemical, or chemical means. For example, useful labels include fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labelled with a fluorescent molecule.

As discussed above the compounds of formula (I) or labelled forms thereof may be used in methods of diagnosis and monitoring in which the method comprises detecting the presence of the label. The detection of the presence of the label is carried out in a manner known in the art and the exact method chosen in each instance will depend upon the identity of the label and the desired detection means. The detection may occur in vivo or in vitro depending upon the aim of the detection or monitoring step. Thus, for example, where a sample from a patient is sent for an autopsy the label is detected in vitro. In contrast in other applications the label may be detected in vivo by scanning the patient to determine the location of the label in the subject such as in radio imaging techniques.

The binding of the compounds of formula (I) to the MC1R may also be used in methods of delivering therapeutic agents to the receptor. The therapeutic agent is typically covalently bound to the receptor and is inherently active at the location of the receptor or it may be an active agent that needs to be activated. An example of an active agent of this type is a radioactive isotope of a metal such as ⁹⁹Tc, ¹¹¹In, I¹²⁵, ⁶⁷Ga, ⁸⁶Y, ⁶⁴Cu, ¹⁸⁸Re and ²¹²Pb, which can be used in radiotherapy applications of diseases associated with abnormal expression of the targeted receptor once the metal has been delivered to the receptor.

Isotopically-labeled compounds of formula (I) can generally be prepared by conventional techniques known to those skilled in the art or by processes analogous to those described in the accompanying Examples and Preparations using appropriate isotopically-labeled reagents in place of the non-labelled reagent previously employed.

Additionally, Formula (I) is intended to cover, where applicable, solvated as well as unsolvated forms of the compounds. Thus, each formula includes compounds having the indicated structure, including the hydrated as well as the non-hydrated forms.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound that undergoes conversion to a compound of formula (I) within a biological system, usually by metabolic means (e.g. by hydrolysis, reduction or oxidation). For example an ester prodrug of a compound of formula (I) containing a hydroxyl group may be convertible by hydrolysis in vivo to the parent molecule. Suitable esters of compounds of formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula (I) containing a carboxy group may be convertible by hydrolysis in vivo to the parent molecule. (Examples of ester prodrugs are those described by F. J. Leinweber, Drug Metab. Res., 18:379, 1987). Similarly, an acyl prodrug of a compound of formula (I) containing an amino group may be convertible by hydrolysis in vivo to the parent molecule (Many examples of prodrugs for these and other functional groups, including amines, are described in Prodrugs: Challenges and Rewards (Parts 1 and 2); Ed V. Stella, R. Borchardt, M. Hageman, R. Oliyai, H. Maag and J Tilley; Springer, 2007).

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The term “functional equivalent” is intended to include variants of the specific receptor described herein. It will be understood that receptors may have isoforms, such that while the primary, secondary, tertiary or quaternary structure of a given receptor isoform is different to the prototypical receptor; the molecule maintains biological activity as a receptor. Isoforms may arise from normal allelic variation within a population and include mutations such as amino acid substitution, deletion, addition, truncation, or duplication. Also included within the term “functional equivalent” are variants generated at the level of transcription.

In the methods and uses of the invention it is observed that certain of the compounds of the Formula (I), are more active than others and therefore it is desirable to use these compounds in the methods and uses of the present invention.

In the methods and uses of the invention a particularly useful subset of compounds of formula (I) are compounds of formula (Ia) as shown below.

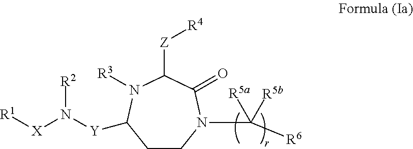

wherein

R¹, R², R³, R^{5a}, R^{5b}, R⁶, X, Y and r are as defined above,

Z is a group of formula —(CR¹³R¹⁴)_{q}—;

R⁴ is selected from the group consisting of H, optionally substituted C₁-C₁₂alkyl, optionally substituted C₂-C₁₂alkenyl, optionally substituted C₂-C₁₂alkynyl, optionally substituted C₃-C₁₂cycloalkyl, optionally substituted C₆-C₁₈aryl, optionally substituted C₁-Cisheteroaryl, NR^{4a}R⁴b, C(═O)R¹⁵, C(═O)NR¹⁶R¹⁷, —C(═NR¹⁶)NR¹⁷R¹⁸, SR²⁰, SC(═O)R²⁰, SO₂R²⁰, OR²⁰, ONR¹⁶R¹⁷, OCR¹⁷R¹⁸R²⁰, OC(═O)R²⁰, OC(═OO)OR²⁰, OC(═O)NR¹⁶R¹⁷, and ONR¹⁶C(═NR¹⁷)NR¹⁸R¹⁹

R^{4a} is selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂heterocycloalkyl, optionally substituted C₆-C₁₈aryl, optionally substituted C₁-C₁₈heteroaryl, C(═O)R^{15a}, C(═O)NR^{15a}R^{16a}, C(═O)OR^{15a}, SO₂R^{15a}, C(═O)H, —C(═NR^{15a})—NR^{16a}R^{17a}, and OR^{15a},

R^{4b} is selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₂heterocycloalkyl, optionally substituted C₆-C₁₈aryl, optionally substituted C₁-Cisheteroaryl, C(═O)R^{15a}, C(═O)NR^{15a}R^{16a}, C(═O)OR^{15a}, or

R^{4a} and R^{4b} when taken together with the nitrogen atom to which they are attached form an optionally substituted heterocyclic moiety, or

one of R^{4a} and R^{4b} when taken together with any R¹³ or R¹⁴ and the atoms to which they are attached forms an optionally substituted heterocyclic moiety;

R¹³ and R¹⁴ are each independently selected from the group consisting of H, halogen, OH, C₁-C₁₂alkyl, C₆-Cisaryl, C₁-C₁₂hydroxyalkyl, C₁-C₁₂haloalkyl, C₁-C₁₂alkyloxy and C₁-C₁₂haloalkyloxy, or

when taken together with the carbon to which they are attached R¹³ and R¹⁴ form an optionally substituted C₃-C₁₂cycloalkyl, or an optionally substituted C₁-C₁₂heterocycloalkyl group, or

one of R¹³ and R¹⁴ when taken together with one of R^{4a}, and R^{4b} and the atoms to which they are attached form an optionally substituted heterocyclic moiety, or

one of R¹³ and R¹⁴ when taken together with one of R¹⁵, R¹⁶, R¹⁷, R¹⁸, R¹⁹ or R²⁰ and the atoms to which they are attached form an optionally substituted cyclic moiety;

each R¹⁵, R^{15a}, R¹⁶, R^{16a}, R¹⁷, R^{17a}, R¹⁸, R¹⁹ and R²⁰ is independently selected from the group consisting of H, optionally substituted C₁-C₁₂alkyl, optionally substituted C₁-C₁₂heteroalkyl, optionally substituted C₃-C₁₂cycloalkyl, optionally substituted C₂-C₁₂ heterocycloalkyl, optionally substituted C₆-C₁₈aryl, and optionally substituted C₁-C₁₈heteroaryl, or

any two of R¹⁵, R^{15a}, R¹⁶, R^{16a}, R¹⁷, R^{17a}, R¹⁸, R¹⁹ and R²⁰ when taken together with the atoms to which they are attached form an optionally substituted cyclic group, or

one of R¹⁵, R¹⁶, R¹⁷, R¹⁸, R¹⁹ and R²⁰ when taken together with one of R¹³ and R¹⁴ and the atoms to which they are attached form an optionally substituted cyclic moiety;

q is an integer selected from the group consisting of 0, 1, 2, 3, 4, and 5;

or a pharmaceutically acceptable salt or prodrug thereof.

In the methods and uses of the invention a particularly useful subset of compounds of formula (I) are compounds where Y is a group of the formula —(CR⁹R¹⁰)ₙ—. In one embodiment of the suitable compounds n is 1 and Y is —CR⁹R¹⁰—. In another embodiment of the suitable compounds n is 2 and Y is —CR⁹R¹⁰CR⁹R¹⁰—.

In one embodiment of the compounds suitable for use in the invention each R⁹ and R¹⁰ is independently selected from H and CH₃. In one specific embodiment R⁹ and R¹⁰ are both H. Accordingly in one embodiment of the compounds suitable for use in the invention Y is —CH₂—. In another embodiment of compounds suitable for use in the invention Y is —CH₂CH₂—. In yet an even further embodiment of compounds suitable for use in the invention Y is —C(CH₃)₂—.

In one embodiment of the compounds suitable for use in the invention R² is H or C₁-C₆ alkyl. In a specific embodiment R² is H.

In one embodiment of the compounds suitable for use in the invention R³ is H or C₁-C₆ alkyl. In a specific embodiment R³ is H.

In one embodiment of the compounds suitable for use in the invention X is selected from the group consisting of —C(═O)— and —(CR¹¹R¹²)ₛ—. In one specific embodiment X is —C(═O)—. In one embodiment of the compounds suitable for use in the invention X is —(CR¹¹R¹²)ₛ—, s is 1. In another embodiment of compounds suitable for use in the invention X is —(CR¹¹R¹²)ₛ—, s is 2. In one form of each of these embodiments R¹¹ and R¹² are each independently selected from the group consisting of H and C₁-C₆ alkyl. In a specific embodiment both R¹¹ and R¹² are H, and s is 1 such that X is —CH₂—.

In one embodiment of the compounds suitable for use in the present invention R=-Z—R⁴, R²═H, R³═H, X═C(═O) and Y═CH₂. This provides compounds of formula (Ib).

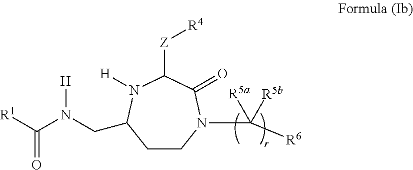

wherein R¹, R⁴, R^{5a}, R^{5b}, R⁶, Z and r are as defined above.

In one embodiment of the compounds suitable for use in the invention and in particular the compounds of formula (Ia) and (Ib) R⁴ is selected from the group consisting of H, C₁-C₁₂alkyl, optionally substituted C₂-C₁₂alkenyl, optionally substituted C₂-C₁₂alkynyl, C₃-C₁₂cycloalkyl, optionally substituted C₆-C₁₈aryl, optionally substituted C-linked C₁-C₁₈heteroaryl, C(═O)R⁵, C(═O)NR¹⁶R, —C(═NR¹⁶)NR¹⁷R¹⁸, SR²⁰, SC(═O)R²⁰, SO₂R²⁰OR²⁰, ONR¹⁶R¹⁷, OCR¹⁷R¹⁸R²⁰, OC(═O)R²⁰, OC(═O)OR²⁰, OC(═O)NR¹⁶R¹⁷, and ONR¹⁶C(═NR¹⁷)NR¹⁸R¹⁹.

In one specific embodiment R⁴ is optionally substituted C₁-C₁₈heteroaryl.

In another embodiment R⁴ is optionally substituted C₃-C₁₂cycloalkyl. In another embodiment R⁴ is C₁-C₁₂alkyl

In another specific embodiment R⁴ is C(═O)NR¹⁶R¹⁷,

In another specific embodiment R⁴ is C(═O)NR¹⁶R¹⁷ and R¹⁶ and R¹⁷ when taken together with the nitrogen atom to which they are attached, form an optionally substituted C₂-C₁₂heterocycloalkyl group. In specific embodiments R¹⁵ and R¹⁶ when taken together with the nitrogen atom to which they are attached form an optionally substituted heterocycloalkyl group selected from the group consisting of piperidin-1-yl, pyrrolidin-1-yl, azetidin-1-yl, azepan-1-yl, morpholin-4-yl, piperazin-1-yl, 4-methyl- and piperazin-1-yl.

In one embodiment of the compounds suitable for use in the invention R¹⁶ is selected from the group consisting of H, CH₃, CH₂CH₃, CH₂CH₂CH₃, CH(CH₃)₂, CH₂CH₂CH₂CH₃, CH(CH₃)CH₂CH₃, CH₂CH(CH₃)₂, C(CH₃)₃, cyclopropyl, cyclobutyl, cyclopentyl, cyclohexyl, benzyl, and phenyl, or a halogenated derivative thereof.

In one embodiment of the compounds suitable for use in the invention R¹⁷ is selected from the group consisting of H, CH₃, CH₂CH₃, CH₂CH₂CH₃, CH(CH₃)₂, CH₂CH₂CH₂CH₃, CH(CH₃)CH₂CH₃, CH₂CH(CH₃)₂, C(CH₃)₃, cyclopropyl, cyclobutyl, cyclopentyl, cyclohexyl, benzyl, and phenyl, or a halogenated derivative thereof.

In one embodiment of the methods and uses of the invention the compound of formula (I) used is one in which R⁴═NR^{4a}R^{4b}. Accordingly a useful subset of compounds for use in the methods and uses of the present invention are compounds of formula (Ic):

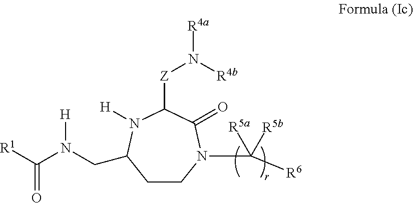

In one embodiment of the compounds suitable for use in the invention, r is selected from the group consisting of 0, 1, 2, 3, and 4. In one specific embodiment r is 0. In another specific embodiment r is 1. In yet a further specific embodiment r is 2. In yet a further specific embodiment r is 3. In an even further specific embodiment r is 4.

In one embodiment of the compounds suitable for use in the invention, and in particular the compounds of formula (I), R^{5a} and R^{5b} are independently selected from H and C₁-C₆ alkyl. In one embodiment R^{5a} and R^{5b} are each independently selected from H and CH₃. In one specific embodiment R^{5a} and R^{5b} are both H.

In one embodiment of the compounds suitable for use in the invention R⁶ is an optionally substituted alkyl group. In one embodiment invention R⁶ is an optionally substituted alkyl group of the formula:

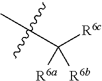

In one embodiment R^{6b} is H.

In one embodiment R^{6a} and R^{6c} are each independently selected from the group consisting of H, optionally substituted C₁-C₁₂alkyl, optionally substituted C₂-C₁₂alkenyl, optionally substituted C₆-Cisaryl and optionally substituted C₁-C₁₈heteroaryl.

In one embodiment R^{6a} and R^{6c} are each independently selected from the group consisting of optionally substituted C₁-C₁₂ alkyl, optionally substituted C₂-C₁₂ alkenyl, optionally substituted C₆-C₁₈ aryl and optionally substituted C₁-C₁₈heteroaryl.

In one embodiment R^{6a} is selected from the group consisting of ethyl, 2,2,2-trifluoroethyl, isopropyl, isopropenyl, propyl, 2-ethyl-propyl, 3,3-dimethyl-propyl, butyl, 2-methyl-butyl, isobutyl, 3,3-dimethyl-butyl, 2-ethyl-butyl, pentyl, 2-methyl-pentyl, optionally substituted phenyl and optionally substituted C₁-C₅ heteroaryl.

In one embodiment R^{6a} is optionally substituted phenyl or optionally substituted C₁-C₁₈heteroaryl.

In one embodiment R^{6c} is selected from the group consisting of ethyl, 2,2,2-trifluoroethyl, isopropyl, isopropenyl, propyl, 2-ethyl-propyl, 3,3-dimethyl-propyl, butyl, 2-methyl-butyl, isobutyl, 3,3-dimethyl-butyl, 2-ethyl-butyl, pentyl, 2-methyl-pentyl, optionally substituted phenyl and optionally substituted C₁-C₅ heteroaryl.

In one embodiment R^{6c} is methyl, ethyl, phenyl or optionally substituted C₁-C₅ heteroaryl.

In the compounds suitable for use in the methods of the present invention Z is a group of formula —(CR¹³R¹⁴)_{q}—. In one embodiment of the compounds suitable for use in the invention, and in particular the compounds of formula (I), formula (Ia), formula (Ib), and formula (Ic), R¹³ and R¹⁴ are independently selected from H and C₁-C₆ alkyl. In one embodiment R¹³ and R¹⁴ are each independently selected from H and CH₃. In one specific embodiment R¹³ and R¹⁴ are both H. In yet another embodiment at least one of R¹³ and R¹⁴ when taken together with at least one of R^{4a} and R^{4b} and the atoms to which they are attached form an optionally substituted heterocycloalkyl group. In one embodiment Z is —(CH₂)_{q}—.

In one embodiment of the compounds suitable for use in the invention q is an integer selected from the group consisting of 0, 1, 2, 3, 4, and 5. In one specific embodiment q is 1. In another specific embodiment q is 2, in yet an even further specific embodiment q is 3, and in yet an even further specific embodiment q is 4.

In one form of the compounds suitable for use in the invention R^{4a} is selected from the group consisting of H, —C(═N)NH₂, —C(═N)N(CH₃)₂, —C(═N)NCH(CH₃)₂, —C(═O)CH₃, —C(═O)cyclohexyl, CH₃, CH₂CH₃, CH₂CH₂CH₃, CH(CH₃)₂, CH₂CH₂CH₂CH₃, CH(CH₃)CH₂CH₃, CH₂CH(CH₃)₂, C(CH₃)₃, cyclopropyl, cyclobutyl, cyclopentyl, cyclohexyl, benzyl, and phenyl, or a halogenated derivative thereof. In one form of the compounds suitable for use in the invention R^{4b} is selected from the group consisting of H, CH₃, CH₂CH₃, CH₂CH₂CH₃, CH(CH₃)₂, CH₂CH₂CH₂CH₃, CH(CH₃)CH₂CH₃, CH₂CH(CH₃)₂, C(CH₃)₃, cyclopropyl, cyclobutyl, cyclopentyl, cyclohexyl, benzyl, and phenyl, or a halogenated derivative thereof.

In another form of the compounds suitable for use in the invention R^{4a} and R^{4b} when taken together with the nitrogen atom to which they are attached form an optionally substituted C₂-C₁₂heterocycloalkyl group, an optionally substituted C₂-C₁₂ heterocycloalkenyl group or an optionally substituted C₁-C₁₈ heteroaryl group.

In a particular embodiment of the compounds suitable for use in the invention R^{4a} and R^{4b} when taken together with the nitrogen atom to which they are attached form an optionally substituted heterocycloalkyl group selected from the group consisting of piperidin-1-yl, pyrrolidin-1-yl, azepan-1-yl, azetidin-1-yl, piperazin-1-yl, morpholin-4-yl, and 4-methyl-piperazin-1-yl.

In one embodiment of the methods and uses of the invention, the compound of formula (I) is one in which one of R^{4a} and R^{4b} when taken together with the nitrogen atom to which it is attached and one of R¹³ and R¹⁴ and the carbon atom to which it is attached form an optionally substituted C₂-C₁₂heterocycloalkyl group. In a particular embodiment one of R^{4a} and R^{4b} when taken together with the nitrogen atom to which it is attached and one of R¹³ and R¹⁴ and the carbon atom to which it is attached form an optionally substituted heterocycloalkyl group selected from the group consisting of piperidinyl, pyrrolidinyl, azepanyl, azetidinyl, morpholinyl, and piperazinyl.

In one specific embodiment of the compounds suitable for use in the invention R¹ is selected from the group consisting of optionally substituted C₂-C₁₂alkenyl, optionally substituted C₆-Cisaryl and optionally substituted C₁-C₁₈heteroaryl.

In one specific embodiment of the compounds suitable for use in the invention R¹ is optionally substituted C₆-Cisaryl. The C₆-Cisaryl may be a monocyclic, bicyclic or polycyclic moiety. In certain embodiments the C₆-C₁₈aryl is a monocyclic moiety. In certain embodiments the C₆-Cisaryl is a bicyclic moiety.

In one specific embodiment R¹ is an optionally substituted C₆-Cisaryl selected from the group consisting of optionally substituted phenyl, biphenyl, and optionally substituted naphthyl. The moieties may be unsubstituted or may be substituted with one or more optional substituents. A wide variety of optional substituents may be used as defined above. Examples of particularly suitable optional substituents include, but are not limited to, F, Br, Cl, methyl, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, cyano, phenoxy, hydroxy, methoxy, ethoxy, pyrrol-1-yl, and 3,5-dimethyl-pyrazol-1-yl.

The substituents may be located at any substitutable position around the aryl ring available for substitution as would be clear to a skilled addressee. Examples of suitable optionally substituted phenyl compounds include, but are not limited to, 2-methoxy-phenyl, 3-methoxy-phenyl, 4-methoxy-phenyl, 2-trifluoromethyl-phenyl, 3-trifluoromethyl-phenyl, 4-trifluoromethyl-phenyl, 2-chloro-phenyl, 3-chloro-phenyl, 4-chloro-phenyl, 4-bromo-phenyl, 2-fluoro-phenyl, 3-fluoro-phenyl, 4-fluoro-phenyl, 4-hydroxy-phenyl, 4-phenyl-phenyl, 4-methyl-phenyl, 2,4-dichloro-phenyl, 3,4-dichloro-phenyl, 2,5-dichloro-phenyl, 2,6-difluoro-phenyl, 2-chloro-6-fluoro-phenyl, 3-fluoro-4-chloro-phenyl, 3-methyl-4-chloro-phenyl, 3-chloro-4-fluoro-phenyl, 3-chloro-4-methyl-phenyl, 2-hydroxy-phenyl, 3-hydroxy-phenyl, 4-hydroxy-phenyl, 4-ethoxy-phenyl, 3-phenoxy-phenyl, 4-phenoxy-phenyl, 2-methyl-phenyl, 3-methyl-phenyl, 4-methyl-phenyl, 4-isopropyl-phenyl, 4-cyano-phenyl 3,4-dimethyl-phenyl, 2,4-dimethyl-phenyl, 4-t-butyl-phenyl, 2,4-dimethoxy-phenyl, and 3,4-methylenedioxy-phenyl.

When R¹ is optionally substituted biphenyl the point of attachment of R¹ to the remainder of the molecule may be at the 2-, 3- or 4-position relative to the point of attachment of the second phenyl ring. As such the biphenyl may be an optionally substituted biphen-2-yl, or an optionally substituted biphen-3-yl, or an optionally substituted biphen-4-yl. In general the optionally substituted biphenyl is an optionally substituted biphen-4-yl. The optionally substituted biphenyl may be substituted in any suitable position.

When R¹ is optionally substituted naphthyl the point of attachment of R¹ to the remainder of the molecule may be at the 1 or 2 position. As such the naphthyl may be an optionally substituted naphth-1-yl, or an optionally substituted naphth-2-yl. In general the optionally substituted naphthyl is an optionally substituted naphth-2-yl. The optionally substituted naphthyl may be substituted in any suitable position. Examples of suitable optionally substituted naphth-2-yls include, but are not limited to, 6-fluoro-naphth-2-yl, 6-bromo-naphth-2-yl, 6-chloro-naphth-2-yl, 1-methoxy-naphth-2-yl, 3-methoxy-naphth-2-yl, 6-methoxy-naphth-2-yl, 1-hydroxy-naphth-2-yl, and 6-amino-naphth-2-yl.

In one specific embodiment of the compounds suitable for use in the invention R¹ is optionally substituted C₁-C₁₈heteroaryl. The C₁-C₁₈heteroaryl may be a monocyclic, bicyclic or polycyclic moiety. In certain embodiments the C₁-C₁₈heteroaryl is a monocyclic moiety. In certain embodiments the C₁-C₁₈heteroaryl is a bicyclic moiety. Examples of suitable heteroaryl moieties include, but are not limited to, indol-2-yl, indol-3-yl quinolin-2-yl quinolin-3-yl, isoquinolin-3-yl, quinoxaline-2-yl, benzo[b]furan-2-yl, benzo[b]thiophen-2-yl, benzo[b]thiophen-5-yl, thiazole-4-yl, benzimidazole-5-yl, benzotriazol-5-yl, furan-2-yl, benzo[d]thiazole-6-yl, pyrazole-1-yl, pyrazole-4-yl and thiophen-2-yl. These may also be optionally substituted as discussed above.

In one specific embodiment of the compounds suitable for use in the invention R¹ is an optionally substituted C₂-C₁₂alkenyl. The optionally substituted alkenyl may contain one or more double bonds with each of the double bonds being independently in the E or Z configuration. In one embodiment of the invention the alkenyl contains a single double bond which is in the E configuration.

In one specific form of this embodiment R¹ is an optionally substituted C₂-C₁₂alkenyl of the formula:

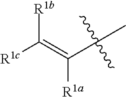

R^{1a} is selected from the group consisting of H, halogen and optionally substituted C₁-C₁₂ alkyl;

R^{1b} and R^{1c} are each independently selected from the group consisting of H, halogen, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂ heterocycloalkyl, optionally substituted C₆-C₁₈aryl, and optionally substituted C₁-C₁₈heteroaryl.

In one form of this embodiment R^{1a} is H. In one form of this embodiment R^{1b} is H. This provides compounds where R¹ is of the formula:

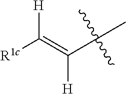

In one embodiment of the compounds of the invention R^{1c} is optionally substituted C₆-C₁₈aryl. The C₆-C₁₈aryl may be monocyclic, bicyclic or polycyclic moiety. In certain embodiments the C₆-C₁₈aryl is a monocyclic moiety. In certain embodiments the C₆-C₁₈aryl is a bicyclic moiety.

In one specific embodiment R^{1c} is an optionally substituted C₆-C₁₈aryl selected from the group consisting of optionally substituted phenyl and optionally substituted naphthyl. The moieties may be unsubstituted or may be substituted with one or more optional substituents. A wide variety of optional substituents may be used as defined above. Examples of particularly suitable optional substituents include, but are not limited to, F, Br, Cl, methyl, trifluoromethyl, ethyl, 2,2,2-trifluoroethyl, isopropyl, propyl, 2-ethyl-propyl, 3,3-dimethyl-propyl, butyl, isobutyl, 3,3-dimethyl-butyl, 2-ethyl-butyl, pentyl, 2-methyl-pentyl, pent-4-enyl, hexyl, heptyl, octyl, phenyl, NH₂, cyano, phenoxy, hydroxy, methoxy, ethoxy, methylenedioxy, pyrrol-1-yl, and 3,5-dimethyl-pyrazol-1-yl.

The substituents may be located at any substitutable position around the aryl ring available for substitution as would be clear to a skilled addressee. Examples of suitable optionally substituted phenyl compounds include, but are not limited to, 2-methoxy-phenyl, 3-methoxy-phenyl, 4-methoxy-phenyl, 2-trifluoromethyl-phenyl, 3-trifluoromethyl-phenyl, 4-trifluoromethyl-phenyl, 2-chloro-phenyl, 3-chloro-phenyl, 4-chloro-phenyl, 4-bromo-phenyl, 2-fluoro-phenyl, 3-fluoro-phenyl, 4-fluoro-phenyl, 4-hydroxy-phenyl, 4-phenyl-phenyl, 4-methyl-phenyl, 2,4-dichloro-phenyl, 3,4-dichloro-phenyl, 2,5-dichloro-phenyl, 2,6-difluoro-phenyl, 2-chloro-6-fluoro-phenyl, 3-fluoro-4-chloro-phenyl, 3-methyl-4-chloro-phenyl, 3-chloro-4-fluoro-phenyl, 3-chloro-4-methyl-phenyl, 2-hydroxy-phenyl, 3-hydroxy-phenyl, 4-hydroxy-phenyl, 4-ethoxy-phenyl, 3-phenoxy-phenyl, 4-phenoxy-phenyl, 2-methyl-phenyl, 3-methyl-phenyl, 4-methyl-phenyl, 4-isopropyl-phenyl, 4-cyano-phenyl 3,4-dimethyl-phenyl, 2,4-dimethyl-phenyl, 4-t-butyl-phenyl, 2,4-dimethoxy-phenyl, and 3,4-methylenedioxy-phenyl.

Specific compounds suitable for use in the methods and uses of the invention include the following:

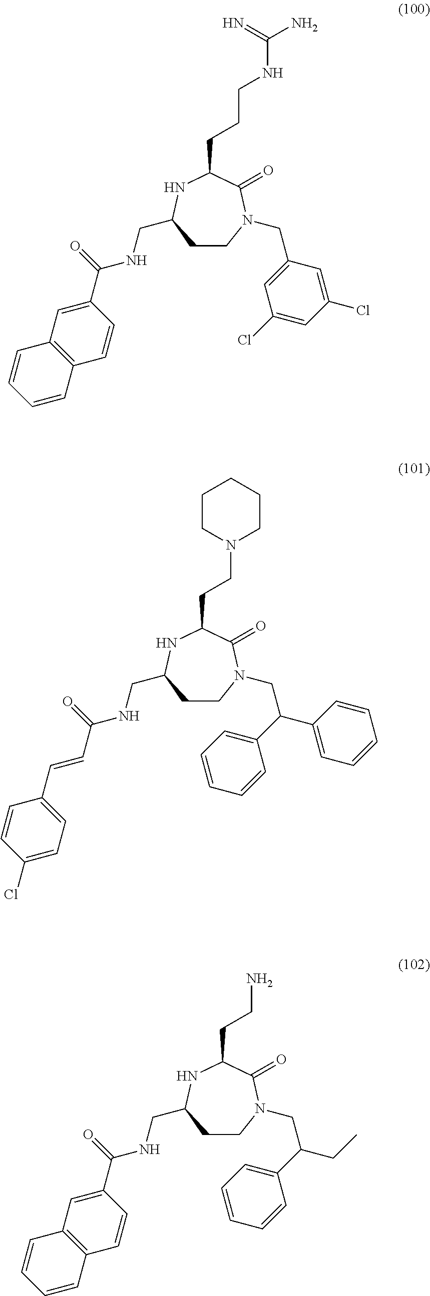 [SMILES: N=C(N)NCCC[C@@H]1N[C@H](CNC(=O)c2ccc3ccccc3c2)CCN(Cc2cc(Cl)cc(Cl)c2)C1=O ; O=C(/C=C/c1ccc(Cl)cc1)NC[C@@H]1CCN(CC(c2ccccc2)c2ccccc2)C(=O)[C@H](CCN2CCCCC2)N1 ; CCC(CN1CC[C@@H](CNC(=O)c2ccc3ccccc3c2)N[C@@H](CCN)C1=O)c1ccccc1]

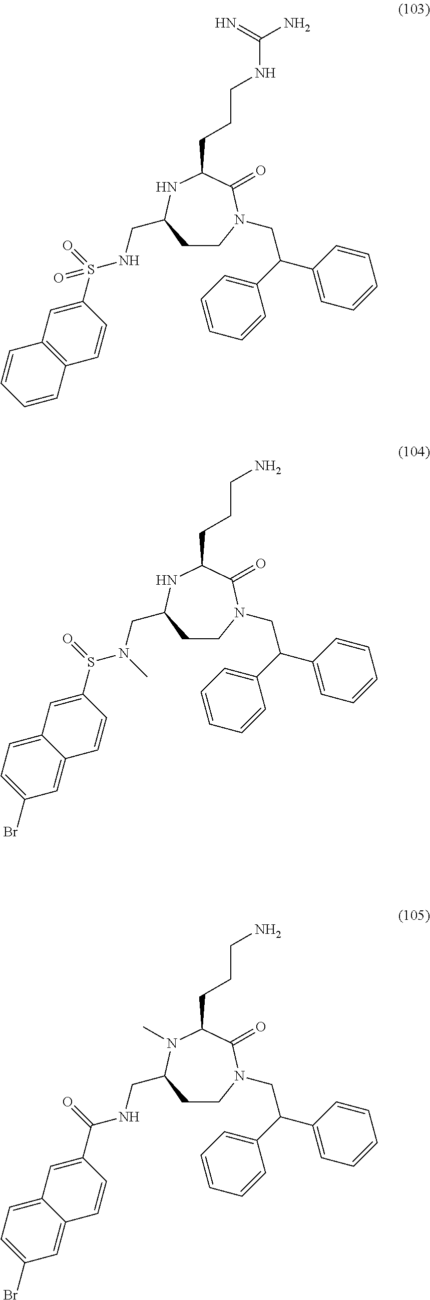 [SMILES: N=C(N)NCCC[C@@H]1N[C@H](CNS(=O)(=O)c2ccc3ccccc3c2)CCN(CC(c2ccccc2)c2ccccc2)C1=O ; CN(C[C@@H]1CCN(CC(c2ccccc2)c2ccccc2)C(=O)[C@H](CCCN)N1)S(=O)c1ccc2cc(Br)ccc2c1 ; CN1[C@H](CNC(=O)c2ccc3cc(Br)ccc3c2)CCN(CC(c2ccccc2)c2ccccc2)C(=O)[C@@H]1CCCN]

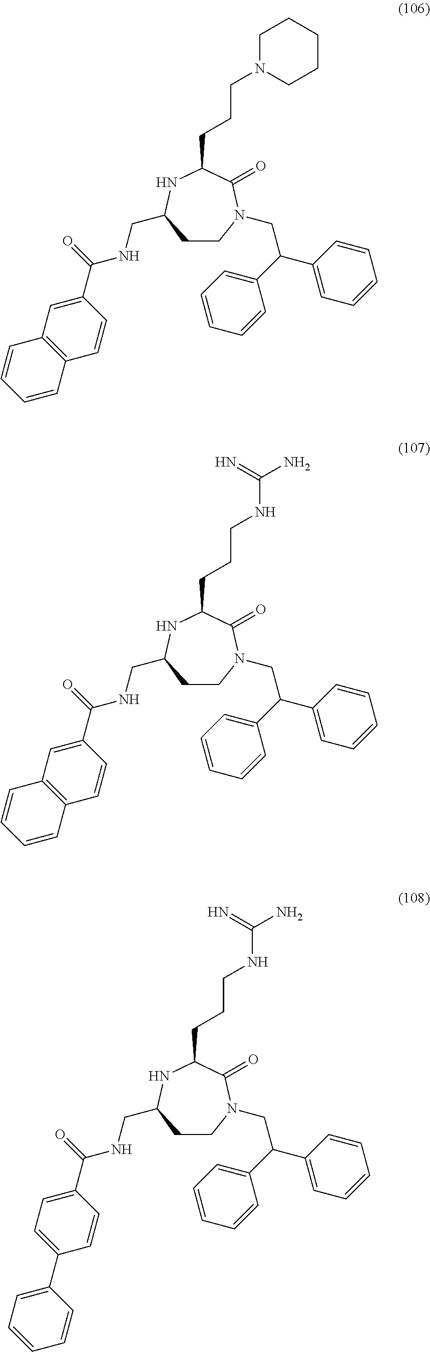 [SMILES: O=C(NC[C@@H]1CCN(CC(c2ccccc2)c2ccccc2)C(=O)[C@H](CCCN2CCCCC2)N1)c1ccc2ccccc2c1 ; N=C(N)NCCC[C@@H]1N[C@H](CNC(=O)c2ccc3ccccc3c2)CCN(CC(c2ccccc2)c2ccccc2)C1=O ; N=C(N)NCCC[C@@H]1N[C@H](CNC(=O)c2ccc(-c3ccccc3)cc2)CCN(CC(c2ccccc2)c2ccccc2)C1=O]

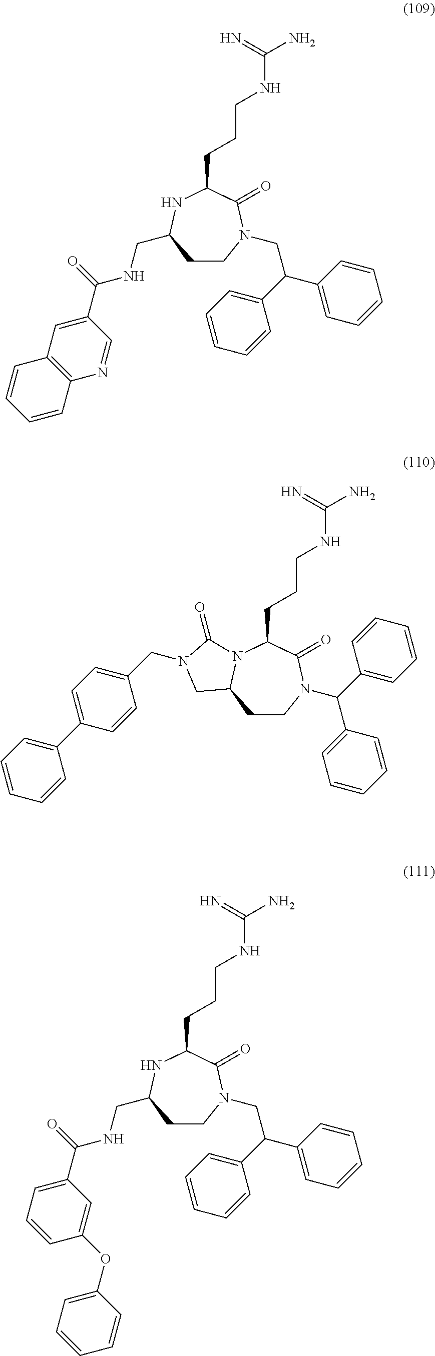 [SMILES: N=C(N)NCCC[C@@H]1N[C@H](CNC(=O)c2cnc3ccccc3c2)CCN(CC(c2ccccc2)c2ccccc2)C1=O ; N=C(N)NCCC[C@H]1C(=O)N(C(c2ccccc2)c2ccccc2)CC[C@H]2CN(Cc3ccc(-c4ccccc4)cc3)C(=O)N21 ; N=C(N)NCCC[C@@H]1N[C@H](CNC(=O)c2cccc(Oc3ccccc3)c2)CCN(CC(c2ccccc2)c2ccccc2)C1=O]

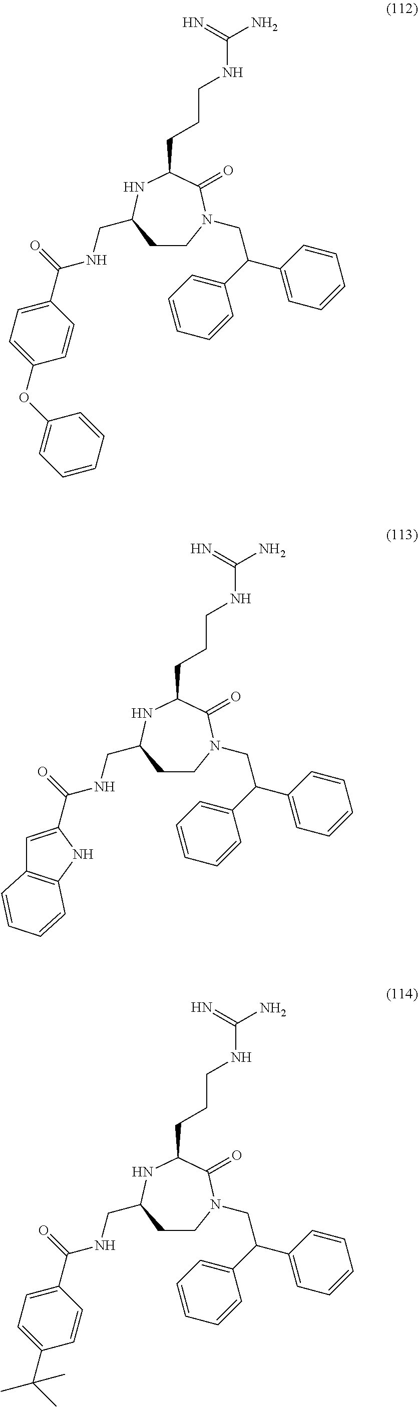 [SMILES: N=C(N)NCCC[C@@H]1N[C@H](CNC(=O)c2ccc(Oc3ccccc3)cc2)CCN(CC(c2ccccc2)c2ccccc2)C1=O ; N=C(N)NCCC[C@@H]1N[C@H](CNC(=O)c2cc3ccccc3[nH]2)CCN(CC(c2ccccc2)c2ccccc2)C1=O ; CC(C)(C)c1ccc(C(=O)NC[C@@H]2CCN(CC(c3ccccc3)c3ccccc3)C(=O)[C@H](CCCNC(=N)N)N2)cc1]

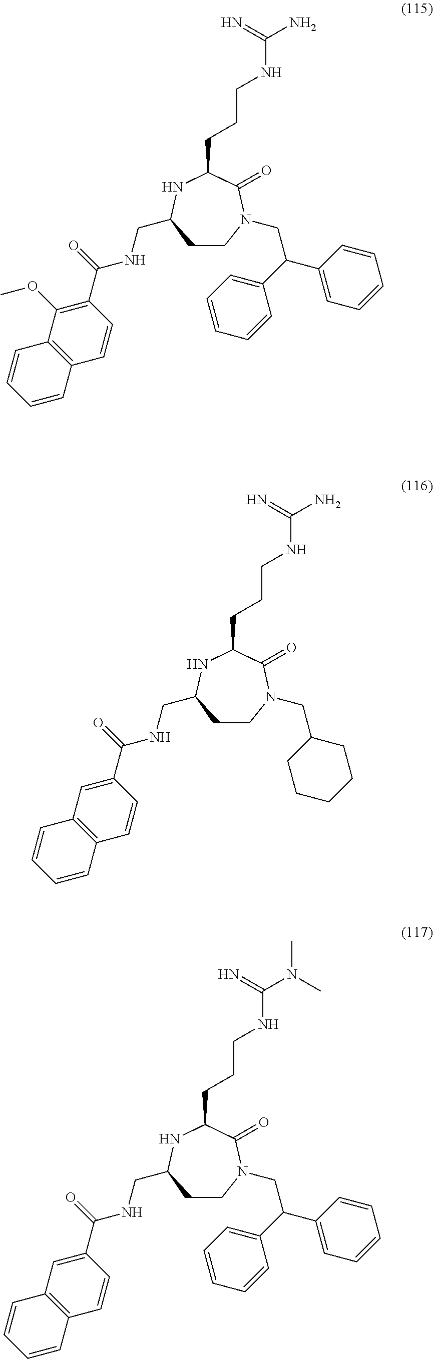 [SMILES: COc1c(C(=O)NC[C@@H]2CCN(CC(c3ccccc3)c3ccccc3)C(=O)[C@H](CCCNC(=N)N)N2)ccc2ccccc12 ; N=C(N)NCCC[C@@H]1N[C@H](CNC(=O)c2ccc3ccccc3c2)CCN(CC2CCCCC2)C1=O ; CN(C)C(=N)NCCC[C@@H]1N[C@H](CNC(=O)c2ccc3ccccc3c2)CCN(CC(c2ccccc2)c2ccccc2)C1=O]

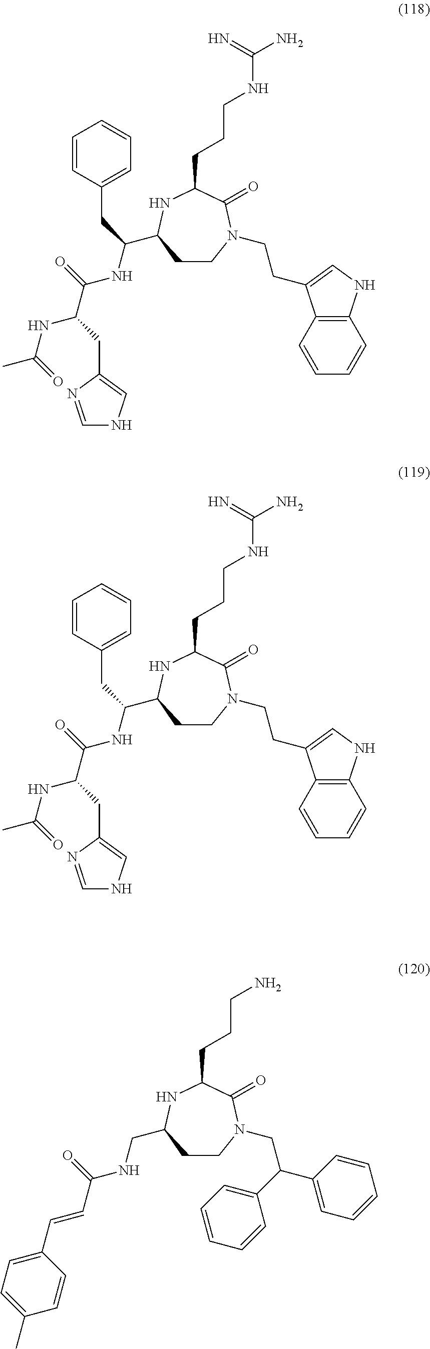 [SMILES: CC(=O)N[C@@H](Cc1c[nH]cn1)C(=O)N[C@@H](Cc1ccccc1)[C@@H]1CCN(CCc2c[nH]c3ccccc23)C(=O)[C@H](CCCNC(=N)N)N1 ; CC(=O)N[C@@H](Cc1c[nH]cn1)C(=O)N[C@H](Cc1ccccc1)[C@@H]1CCN(CCc2c[nH]c3ccccc23)C(=O)[C@H](CCCNC(=N)N)N1 ; Cc1ccc(/C=C/C(=O)NC[C@@H]2CCN(CC(c3ccccc3)c3ccccc3)C(=O)[C@H](CCCN)N2)cc1]

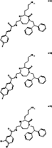 [SMILES: NCCC[C@@H]1N[C@H](CNC(=O)/C=C/c2ccc(F)cc2)CCN(CC(c2ccccc2)c2ccccc2)C1=O ; NCCC[C@@H]1N[C@H](CNC(=O)c2ccc3cc(F)ccc3c2)CCN(CC(c2ccccc2)c2ccccc2)C1=O ; NCCC[C@@H]1N[C@H](CNC(=O)c2ccc(Cl)c(Cl)c2)CCN(CC(c2ccccc2)c2ccccc2)C1=O]

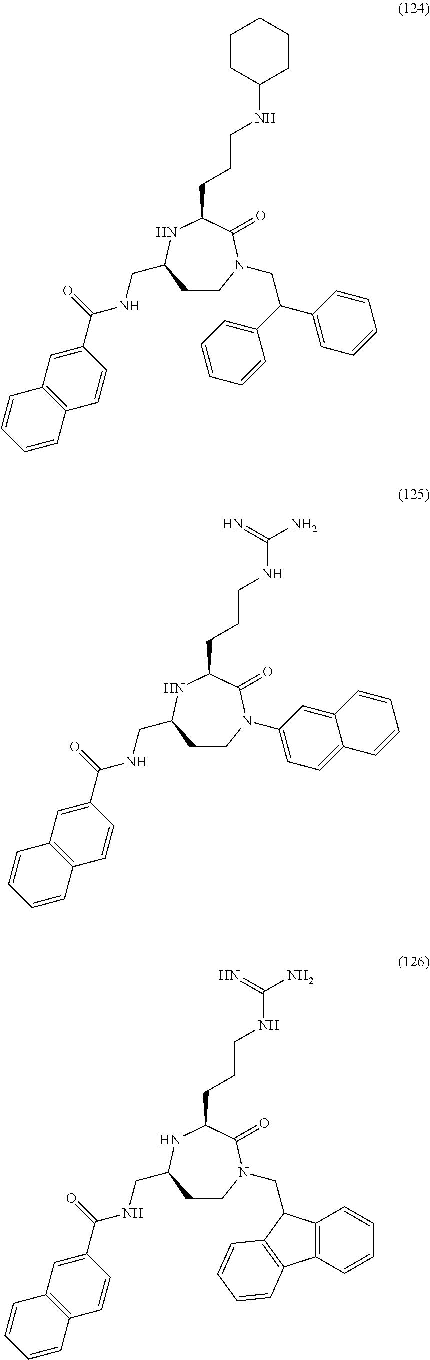 [SMILES: O=C(NC[C@@H]1CCN(CC(c2ccccc2)c2ccccc2)C(=O)[C@H](CCCNC2CCCCC2)N1)c1ccc2ccccc2c1 ; N=C(N)NCCC[C@@H]1N[C@H](CNC(=O)c2ccc3ccccc3c2)CCN(c2ccc3ccccc3c2)C1=O ; N=C(N)NCCC[C@@H]1N[C@H](CNC(=O)c2ccc3ccccc3c2)CCN(CC2c3ccccc3-c3ccccc32)C1=O]

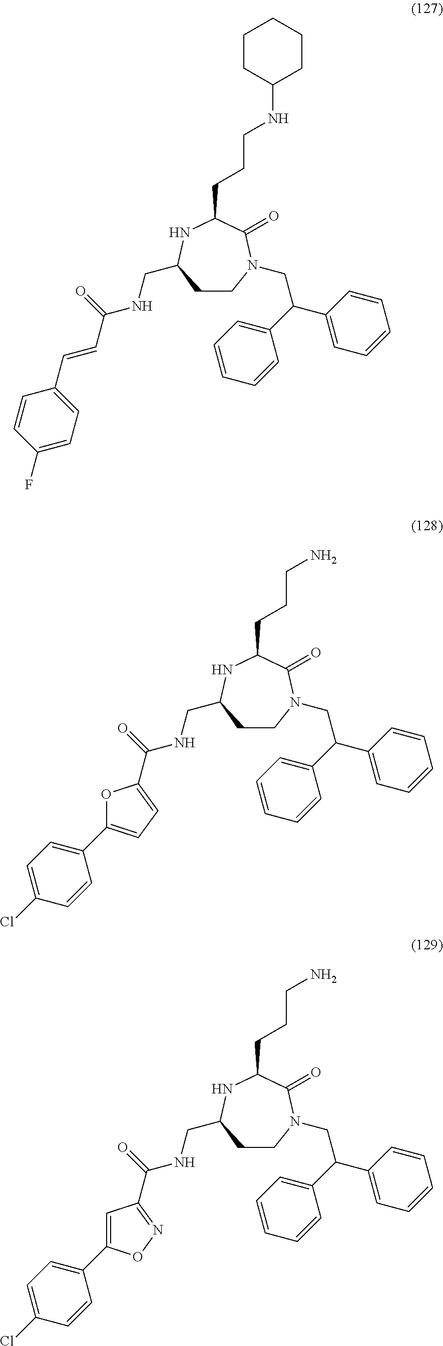 [SMILES: O=C(/C=C/c1ccc(F)cc1)NC[C@@H]1CCN(CC(c2ccccc2)c2ccccc2)C(=O)[C@H](CCCNC2CCCCC2)N1 ; NCCC[C@@H]1N[C@H](CNC(=O)c2ccc(-c3ccc(Cl)cc3)o2)CCN(CC(c2ccccc2)c2ccccc2)C1=O ; NCCC[C@@H]1N[C@H](CNC(=O)c2cc(-c3ccc(Cl)cc3)on2)CCN(CC(c2ccccc2)c2ccccc2)C1=O]

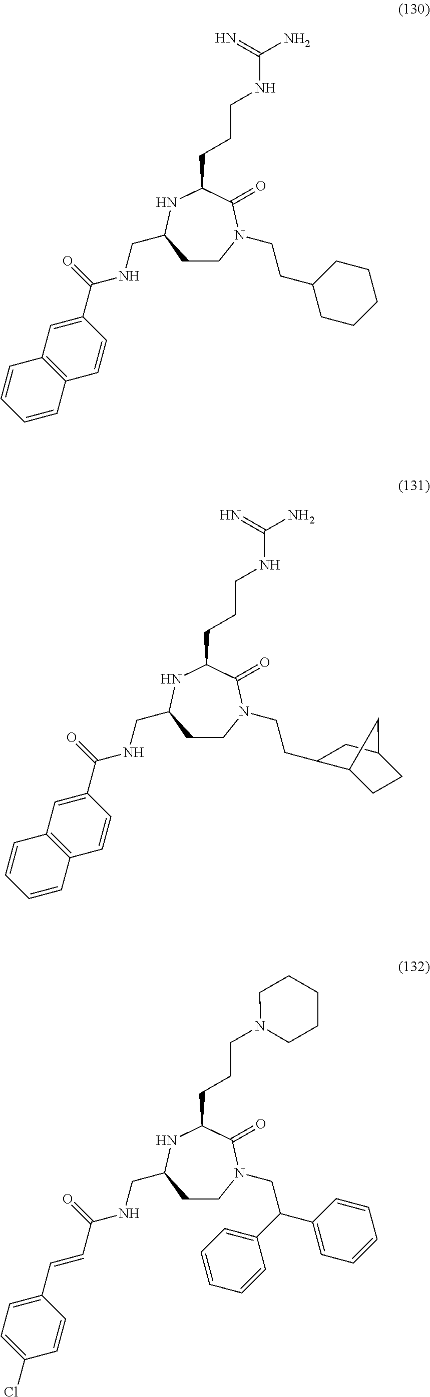 [SMILES: N=C(N)NCCC[C@@H]1N[C@H](CNC(=O)c2ccc3ccccc3c2)CCN(CCC2CCCCC2)C1=O ; N=C(N)NCCC[C@@H]1N[C@H](CNC(=O)c2ccc3ccccc3c2)CCN(CCC2CC3CCC2C3)C1=O ; O=C(/C=C/c1ccc(Cl)cc1)NC[C@@H]1CCN(CC(c2ccccc2)c2ccccc2)C(=O)[C@H](CCCN2CCCCC2)N1]

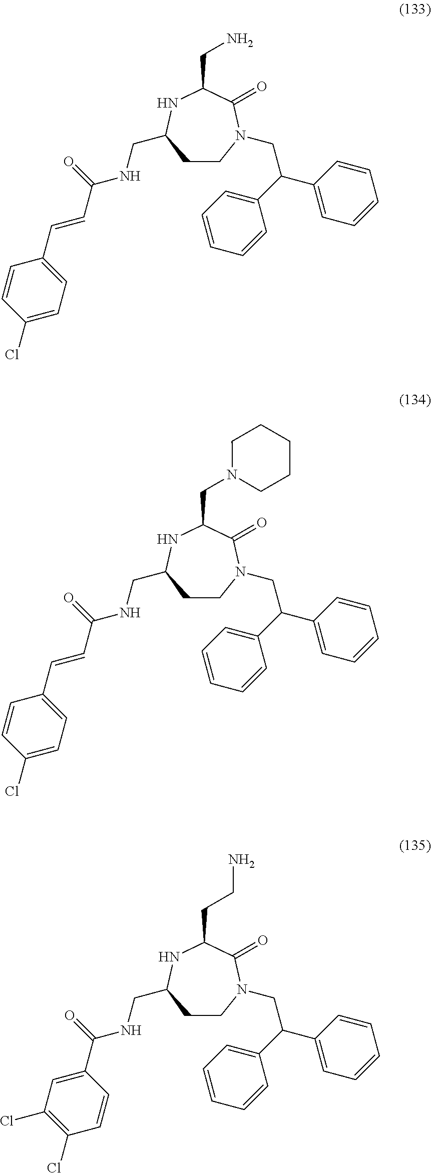 [SMILES: NC[C@@H]1N[C@H](CNC(=O)/C=C/c2ccc(Cl)cc2)CCN(CC(c2ccccc2)c2ccccc2)C1=O ; O=C(/C=C/c1ccc(Cl)cc1)NC[C@@H]1CCN(CC(c2ccccc2)c2ccccc2)C(=O)[C@H](CN2CCCCC2)N1 ; NCC[C@@H]1N[C@H](CNC(=O)c2ccc(Cl)c(Cl)c2)CCN(CC(c2ccccc2)c2ccccc2)C1=O]

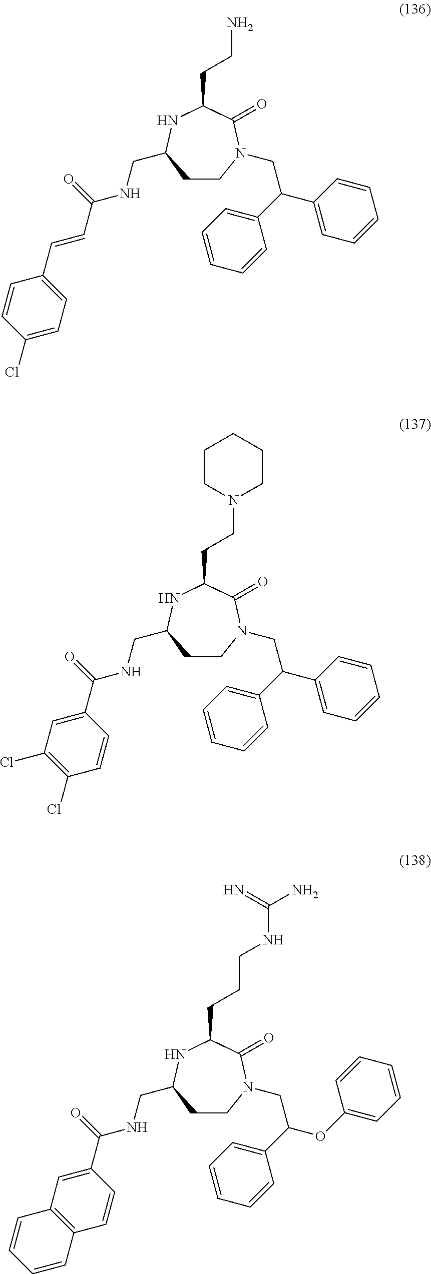 [SMILES: NCC[C@@H]1N[C@H](CNC(=O)/C=C/c2ccc(Cl)cc2)CCN(CC(c2ccccc2)c2ccccc2)C1=O ; O=C(NC[C@@H]1CCN(CC(c2ccccc2)c2ccccc2)C(=O)[C@H](CCN2CCCCC2)N1)c1ccc(Cl)c(Cl)c1 ; N=C(N)NCCC[C@@H]1N[C@H](CNC(=O)c2ccc3ccccc3c2)CCN(CC(Oc2ccccc2)c2ccccc2)C1=O]

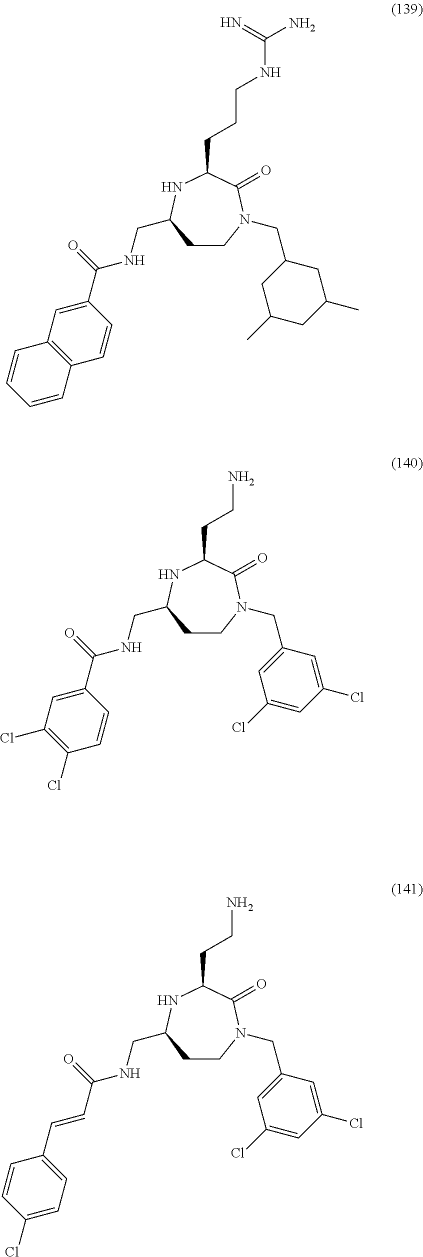 [SMILES: CC1CC(C)CC(CN2CC[C@@H](CNC(=O)c3ccc4ccccc4c3)N[C@@H](CCCNC(=N)N)C2=O)C1 ; NCC[C@@H]1N[C@H](CNC(=O)c2ccc(Cl)c(Cl)c2)CCN(Cc2cc(Cl)cc(Cl)c2)C1=O ; NCC[C@@H]1N[C@H](CNC(=O)/C=C/c2ccc(Cl)cc2)CCN(Cc2cc(Cl)cc(Cl)c2)C1=O]

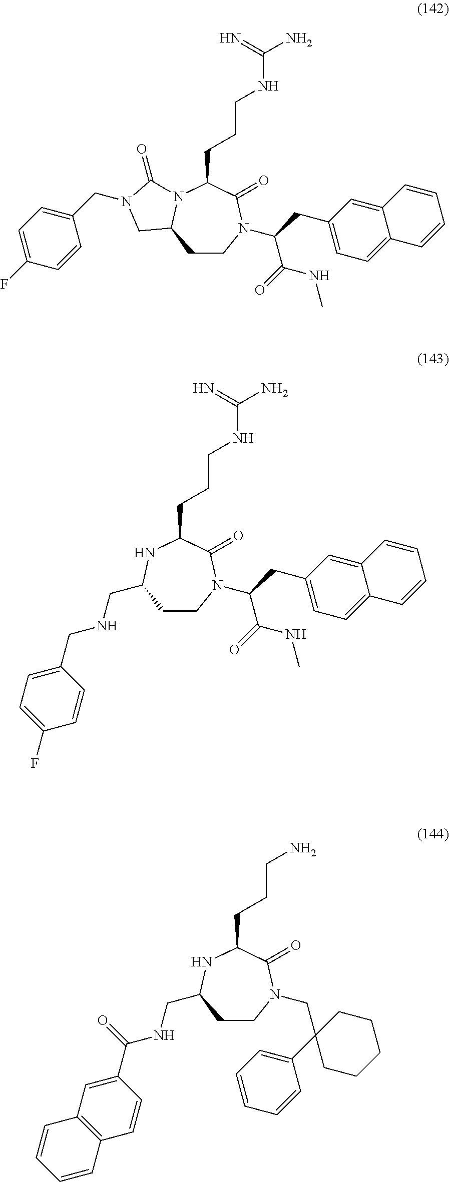 [SMILES: CNC(=O)[C@H](Cc1ccc2ccccc2c1)N1CC[C@H]2CN(Cc3ccc(F)cc3)C(=O)N2[C@@H](CCCNC(=N)N)C1=O ; CNC(=O)[C@H](Cc1ccc2ccccc2c1)N1CC[C@H](CNCc2ccc(F)cc2)N[C@@H](CCCNC(=N)N)C1=O ; NCCC[C@@H]1N[C@H](CNC(=O)c2ccc3ccccc3c2)CCN(CC2(c3ccccc3)CCCCC2)C1=O]

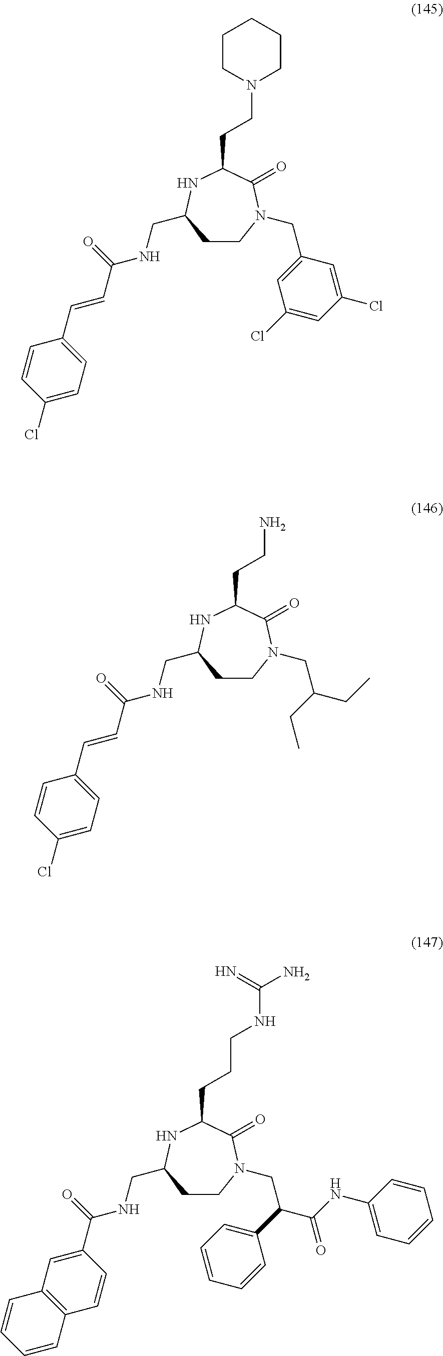 [SMILES: O=C(/C=C/c1ccc(Cl)cc1)NC[C@@H]1CCN(Cc2cc(Cl)cc(Cl)c2)C(=O)[C@H](CCN2CCCCC2)N1 ; CCC(CC)CN1CC[C@@H](CNC(=O)/C=C/c2ccc(Cl)cc2)N[C@@H](CCN)C1=O ; N=C(N)NCCC[C@@H]1N[C@H](CNC(=O)c2ccc3ccccc3c2)CCN(C[C@@H](C(=O)Nc2ccccc2)c2ccccc2)C1=O]

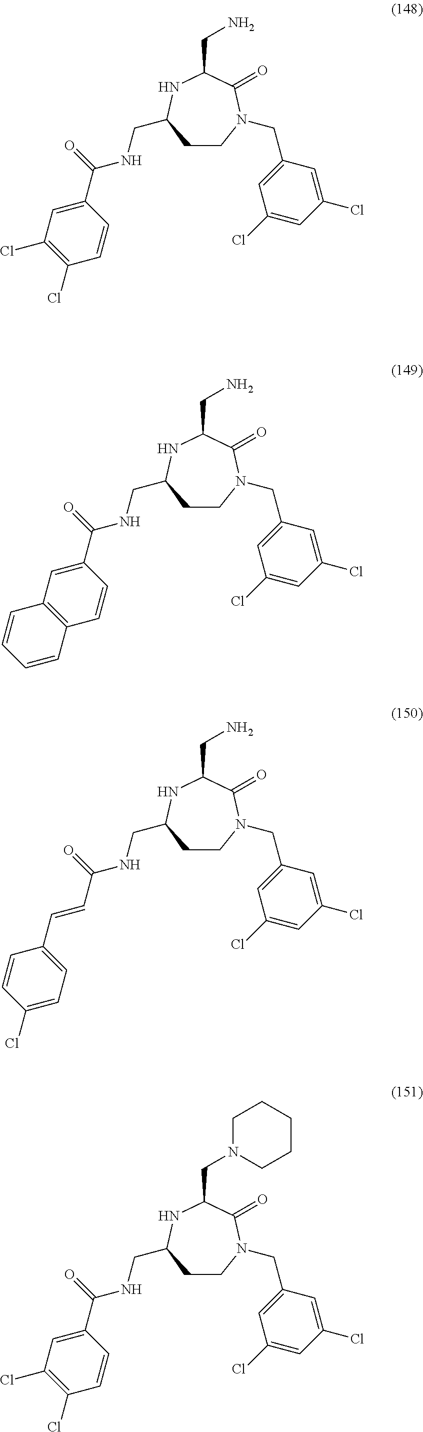 [SMILES: NC[C@@H]1N[C@H](CNC(=O)c2ccc(Cl)c(Cl)c2)CCN(Cc2cc(Cl)cc(Cl)c2)C1=O ; NC[C@@H]1N[C@H](CNC(=O)c2ccc3ccccc3c2)CCN(Cc2cc(Cl)cc(Cl)c2)C1=O ; NC[C@@H]1N[C@H](CNC(=O)/C=C/c2ccc(Cl)cc2)CCN(Cc2cc(Cl)cc(Cl)c2)C1=O ; O=C(NC[C@@H]1CCN(Cc2cc(Cl)cc(Cl)c2)C(=O)[C@H](CN2CCCCC2)N1)c1ccc(Cl)c(Cl)c1]

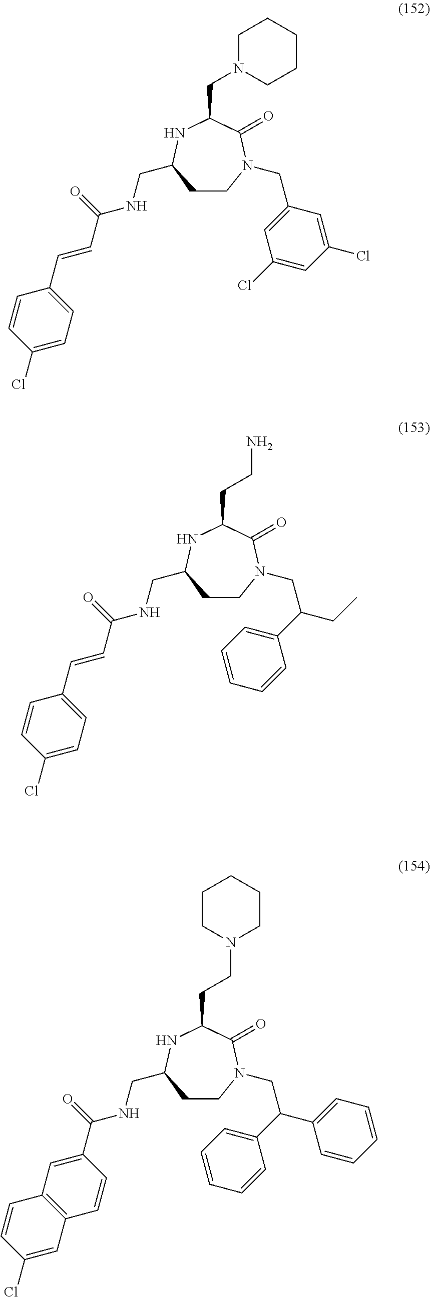 [SMILES: O=C(/C=C/c1ccc(Cl)cc1)NC[C@@H]1CCN(Cc2cc(Cl)cc(Cl)c2)C(=O)[C@H](CN2CCCCC2)N1 ; CCC(CN1CC[C@@H](CNC(=O)/C=C/c2ccc(Cl)cc2)N[C@@H](CCN)C1=O)c1ccccc1 ; O=C(NC[C@@H]1CCN(CC(c2ccccc2)c2ccccc2)C(=O)[C@H](CCN2CCCCC2)N1)c1ccc2cc(Cl)ccc2c1]

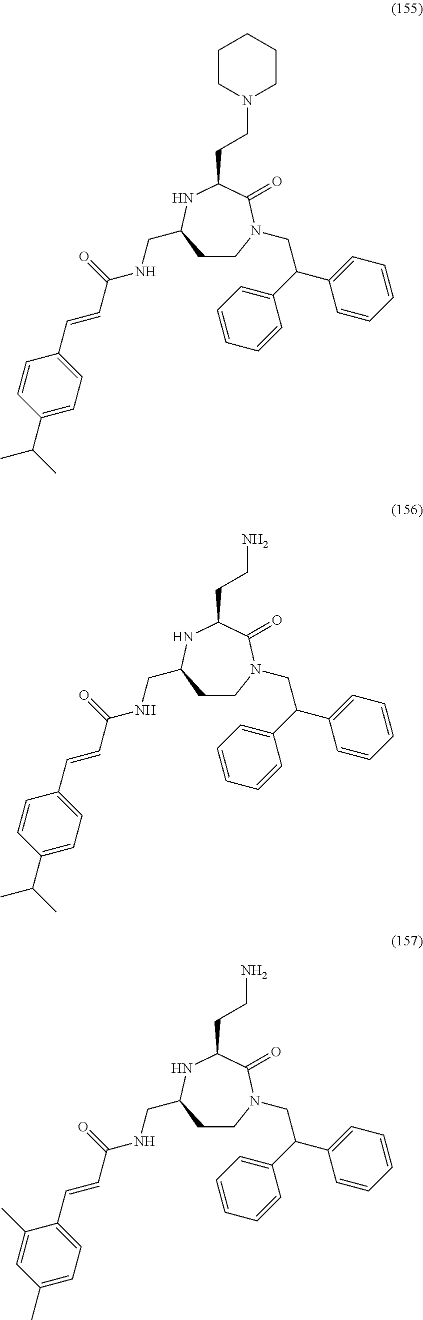 [SMILES: CC(C)c1ccc(/C=C/C(=O)NC[C@@H]2CCN(CC(c3ccccc3)c3ccccc3)C(=O)[C@H](CCN3CCCCC3)N2)cc1 ; CC(C)c1ccc(/C=C/C(=O)NC[C@@H]2CCN(CC(c3ccccc3)c3ccccc3)C(=O)[C@H](CCN)N2)cc1 ; Cc1ccc(/C=C/C(=O)NC[C@@H]2CCN(CC(c3ccccc3)c3ccccc3)C(=O)[C@H](CCN)N2)c(C)c1]

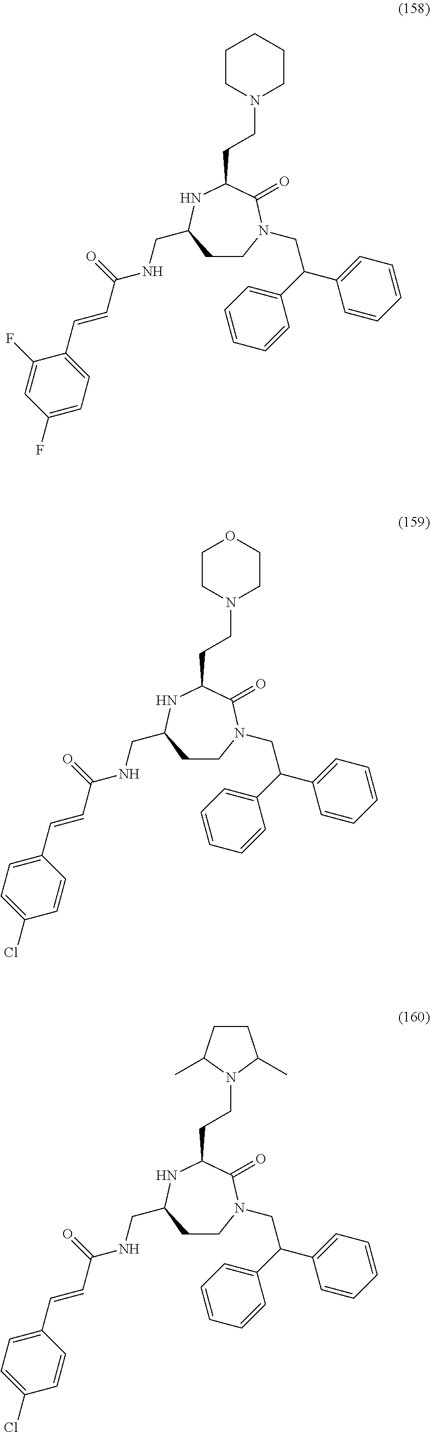 [SMILES: O=C(/C=C/c1ccc(F)cc1F)NC[C@@H]1CCN(CC(c2ccccc2)c2ccccc2)C(=O)[C@H](CCN2CCCCC2)N1 ; O=C(/C=C/c1ccc(Cl)cc1)NC[C@@H]1CCN(CC(c2ccccc2)c2ccccc2)C(=O)[C@H](CCN2CCOCC2)N1 ; CC1CCC(C)N1CC[C@@H]1N[C@H](CNC(=O)/C=C/c2ccc(Cl)cc2)CCN(CC(c2ccccc2)c2ccccc2)C1=O]

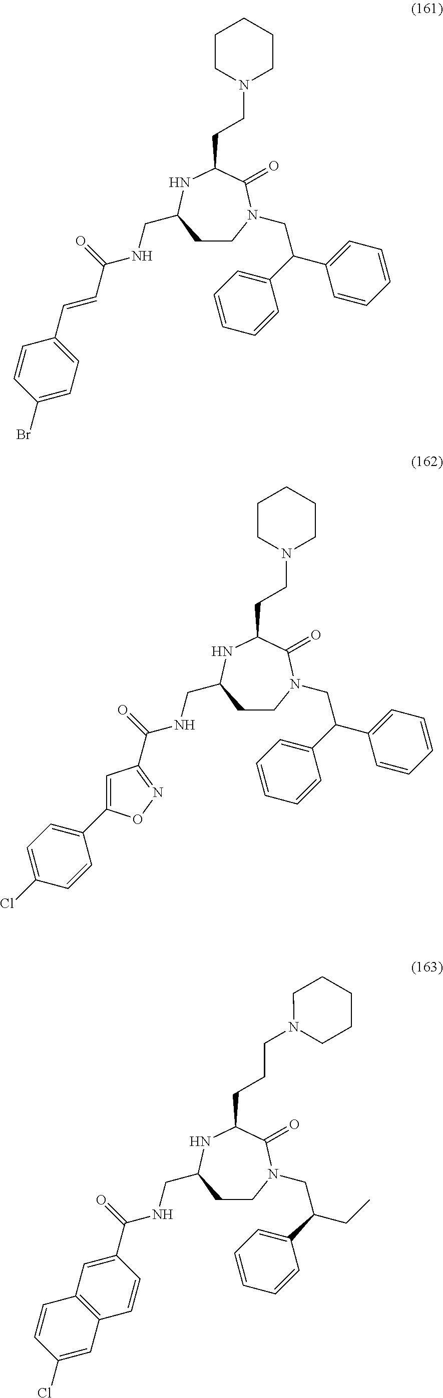 [SMILES: O=C(/C=C/c1ccc(Br)cc1)NC[C@@H]1CCN(CC(c2ccccc2)c2ccccc2)C(=O)[C@H](CCN2CCCCC2)N1 ; O=C(NC[C@@H]1CCN(CC(c2ccccc2)c2ccccc2)C(=O)[C@H](CCN2CCCCC2)N1)c1cc(-c2ccc(Cl)cc2)on1 ; CC[C@H](CN1CC[C@@H](CNC(=O)c2ccc3cc(Cl)ccc3c2)N[C@@H](CCCN2CCCCC2)C1=O)c1ccccc1]

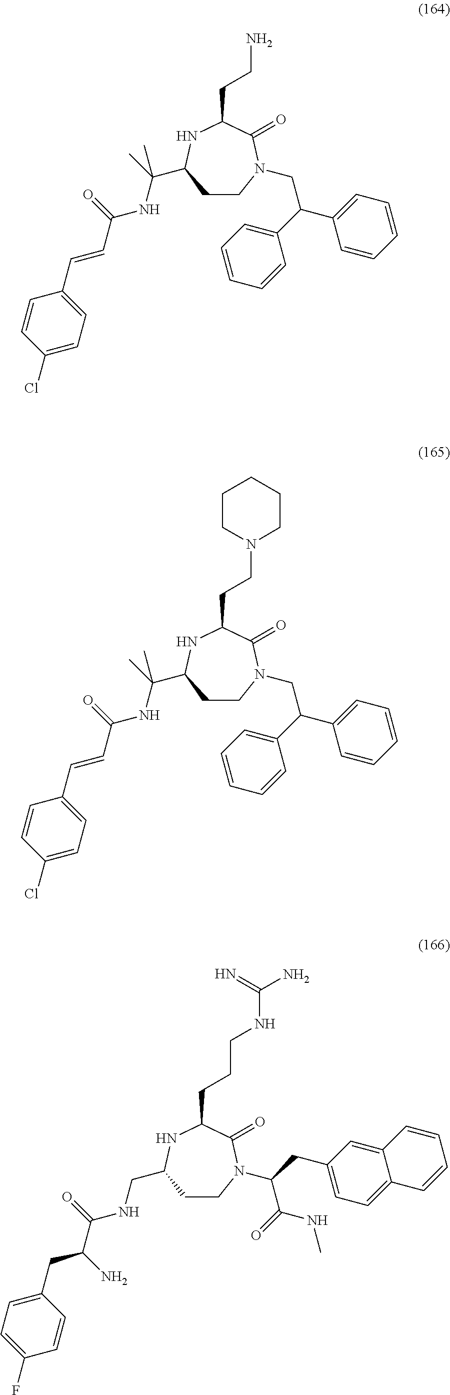 [SMILES: CC(C)(NC(=O)/C=C/c1ccc(Cl)cc1)[C@@H]1CCN(CC(c2ccccc2)c2ccccc2)C(=O)[C@H](CCN)N1 ; CC(C)(NC(=O)/C=C/c1ccc(Cl)cc1)[C@@H]1CCN(CC(c2ccccc2)c2ccccc2)C(=O)[C@H](CCN2CCCCC2)N1 ; CNC(=O)[C@H](Cc1ccc2ccccc2c1)N1CC[C@H](CNC(=O)[C@@H](N)Cc2ccc(F)cc2)N[C@@H](CCCNC(=N)N)C1=O]

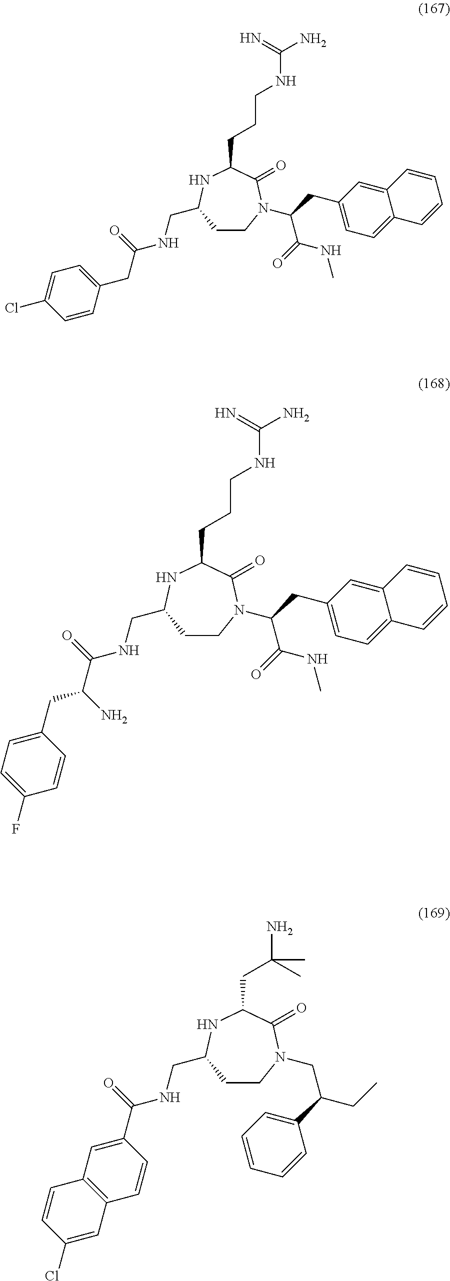 [SMILES: CNC(=O)[C@H](Cc1ccc2ccccc2c1)N1CC[C@H](CNC(=O)Cc2ccc(Cl)cc2)N[C@@H](CCCNC(=N)N)C1=O ; CNC(=O)[C@H](Cc1ccc2ccccc2c1)N1CC[C@H](CNC(=O)[C@H](N)Cc2ccc(F)cc2)N[C@@H](CCCNC(=N)N)C1=O ; CC[C@H](CN1CC[C@H](CNC(=O)c2ccc3cc(Cl)ccc3c2)N[C@H](CC(C)(C)N)C1=O)c1ccccc1]

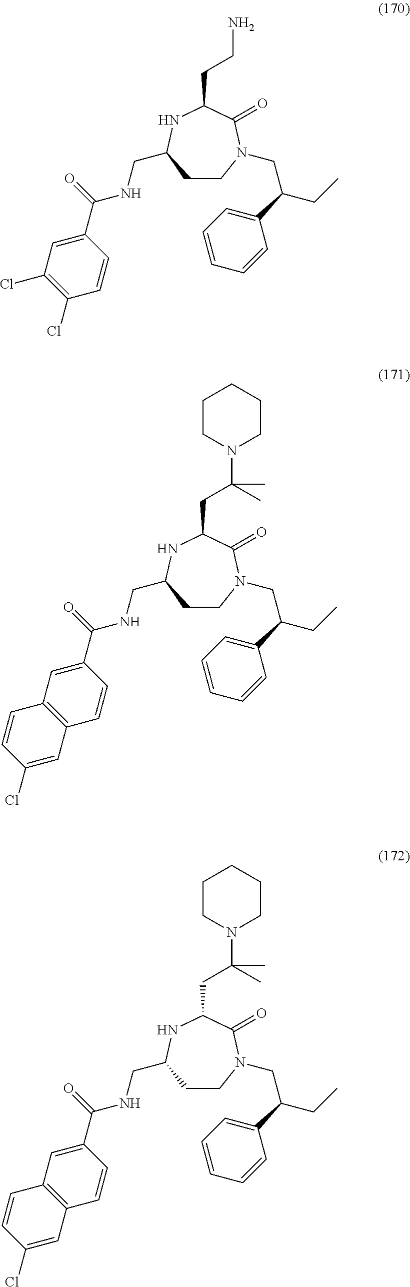 [SMILES: CC[C@H](CN1CC[C@@H](CNC(=O)c2ccc(Cl)c(Cl)c2)N[C@@H](CCN)C1=O)c1ccccc1 ; CC[C@H](CN1CC[C@@H](CNC(=O)c2ccc3cc(Cl)ccc3c2)N[C@@H](CC(C)(C)N2CCCCC2)C1=O)c1ccccc1 ; CC[C@H](CN1CC[C@H](CNC(=O)c2ccc3cc(Cl)ccc3c2)N[C@H](CC(C)(C)N2CCCCC2)C1=O)c1ccccc1]

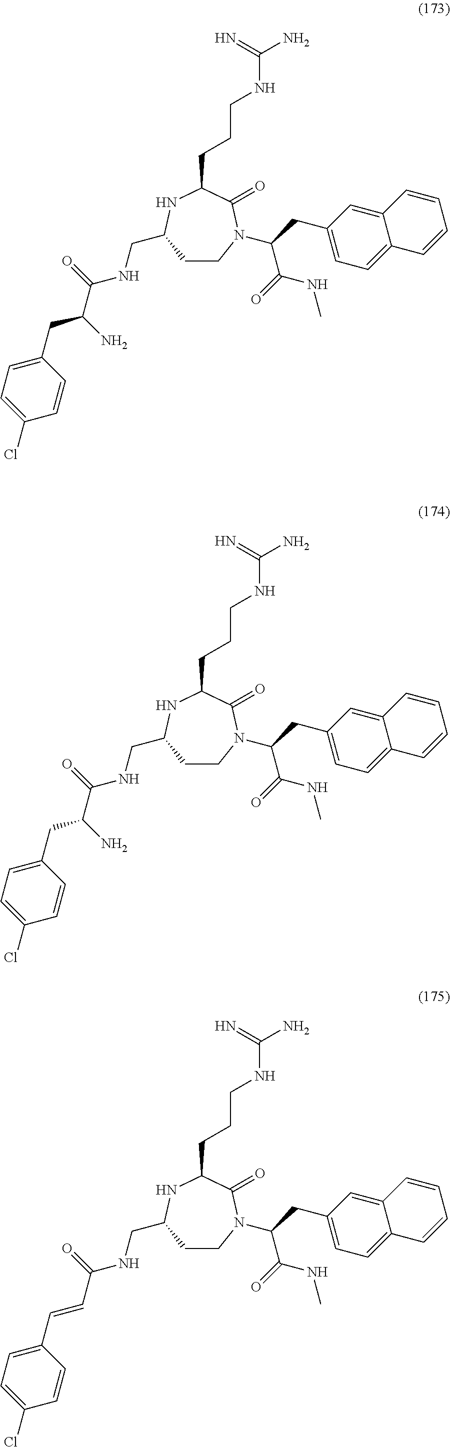 [SMILES: CNC(=O)[C@H](Cc1ccc2ccccc2c1)N1CC[C@H](CNC(=O)[C@@H](N)Cc2ccc(Cl)cc2)N[C@@H](CCCNC(=N)N)C1=O ; CNC(=O)[C@H](Cc1ccc2ccccc2c1)N1CC[C@H](CNC(=O)[C@H](N)Cc2ccc(Cl)cc2)N[C@@H](CCCNC(=N)N)C1=O ; CNC(=O)[C@H](Cc1ccc2ccccc2c1)N1CC[C@H](CNC(=O)/C=C/c2ccc(Cl)cc2)N[C@@H](CCCNC(=N)N)C1=O]

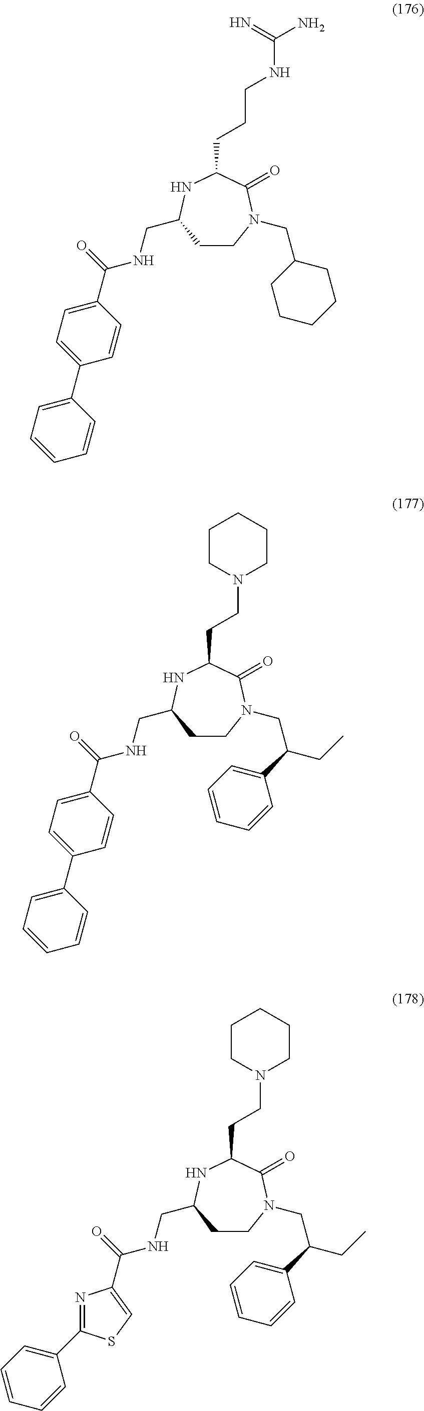 [SMILES: N=C(N)NCCC[C@H]1N[C@@H](CNC(=O)c2ccc(-c3ccccc3)cc2)CCN(CC2CCCCC2)C1=O ; CC[C@H](CN1CC[C@@H](CNC(=O)c2ccc(-c3ccccc3)cc2)N[C@@H](CCN2CCCCC2)C1=O)c1ccccc1 ; CC[C@H](CN1CC[C@@H](CNC(=O)c2csc(-c3ccccc3)n2)N[C@@H](CCN2CCCCC2)C1=O)c1ccccc1]

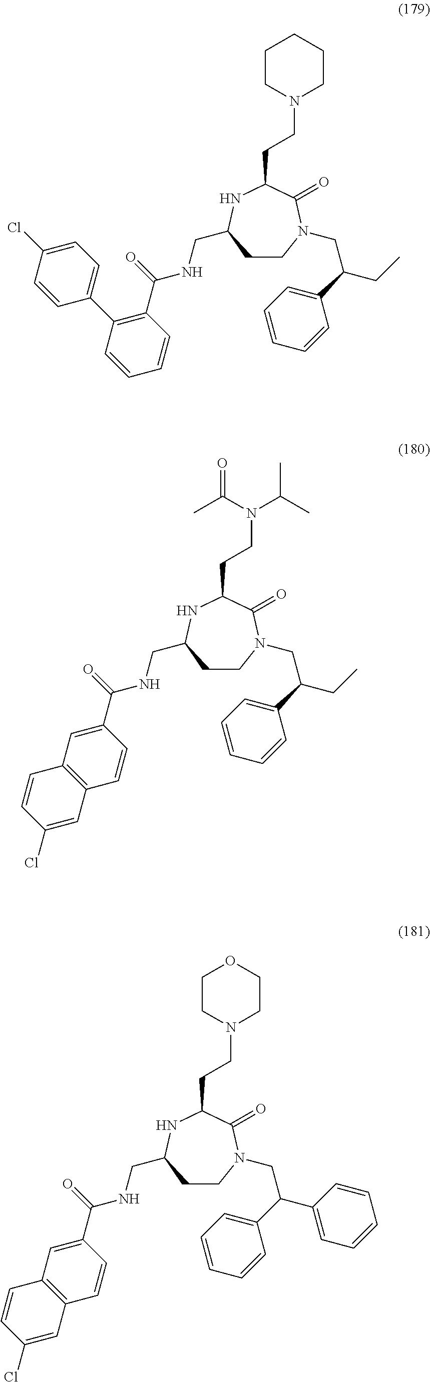 [SMILES: CC[C@H](CN1CC[C@@H](CNC(=O)c2ccccc2-c2ccc(Cl)cc2)N[C@@H](CCN2CCCCC2)C1=O)c1ccccc1 ; CC[C@H](CN1CC[C@@H](CNC(=O)c2ccc3cc(Cl)ccc3c2)N[C@@H](CCN(C(C)=O)C(C)C)C1=O)c1ccccc1 ; O=C(NC[C@@H]1CCN(CC(c2ccccc2)c2ccccc2)C(=O)[C@H](CCN2CCOCC2)N1)c1ccc2cc(Cl)ccc2c1]

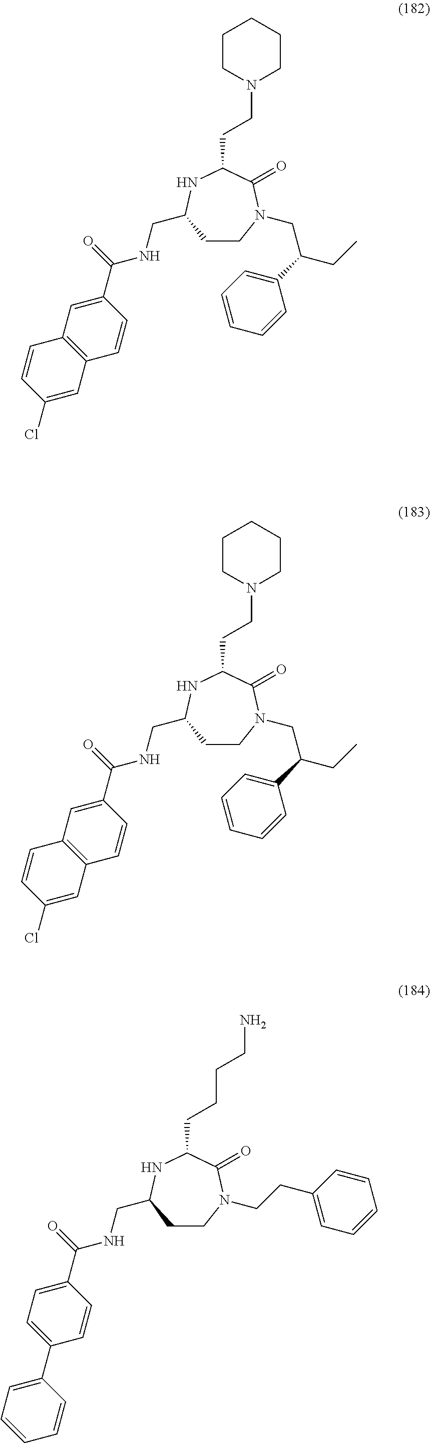 [SMILES: CC[C@@H](CN1CC[C@H](CNC(=O)c2ccc3cc(Cl)ccc3c2)N[C@H](CCN2CCCCC2)C1=O)c1ccccc1 ; CC[C@H](CN1CC[C@H](CNC(=O)c2ccc3cc(Cl)ccc3c2)N[C@H](CCN2CCCCC2)C1=O)c1ccccc1 ; NCCCC[C@H]1N[C@H](CNC(=O)c2ccc(-c3ccccc3)cc2)CCN(CCc2ccccc2)C1=O]

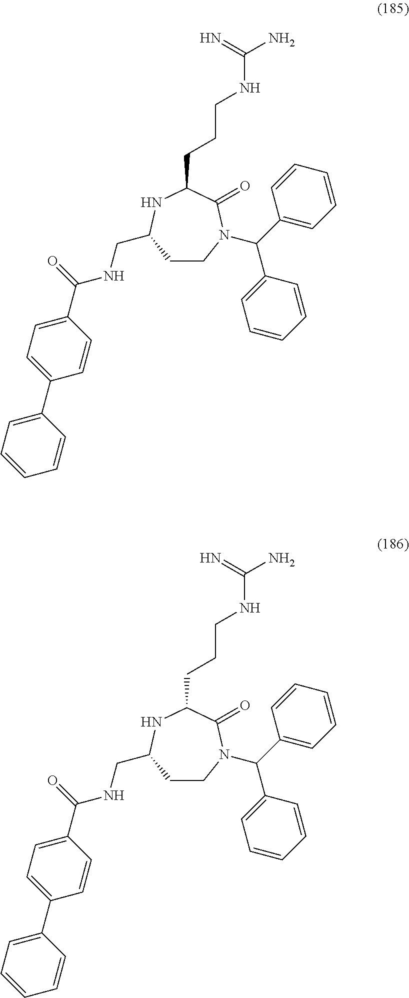 [SMILES: N=C(N)NCCC[C@@H]1N[C@@H](CNC(=O)c2ccc(-c3ccccc3)cc2)CCN(C(c2ccccc2)c2ccccc2)C1=O ; N=C(N)NCCC[C@H]1N[C@@H](CNC(=O)c2ccc(-c3ccccc3)cc2)CCN(C(c2ccccc2)c2ccccc2)C1=O]

or a pharmaceutically acceptable salt or prodrug thereof.

In order to assist the reader the names of compounds suitable for use in the invention as discussed above are as follows:
(100) N-(((3S,5S)-1-(3,5-dichlorobenzyl)-3-(3-guanidinopropyl)-2-oxo-1,4-diazepan-5-yl)methyl)-2-naphthamide
(101) (E)-3-(4-chlorophenyl)-N-(((3S,5S)-1-(2,2-diphenylethyl)-2-oxo-3-(2-(piperidin-1-yl)ethyl)-1,4-diazepan-5-yl)methyl)acrylamide
(102) N-(((3S,5S)-3-(2-aminoethyl)-2-oxo-1-(2-phenylbutyl)-1,4-diazepan-5-yl)methyl)-2-naphthamide
(103) N-(((3S,5S)-1-(2,2-diphenylethyl)-3-(3-guanidinopropyl)-2-oxo-1,4-diazepan-5-yl)methyl)naphthalene-2-sulfonamide
(104) N-(((3S,5S)-3-(3-aminopropyl)-1-(2,2-diphenylethyl)-2-oxo-1,4-diazepan-5-yl)methyl)-6-bromo-N-methyl-2-naphthamide
(105) N-(((3S,5S)-3-(3-aminopropyl)-1-(2,2-diphenylethyl)-4-methyl-2-oxo-1,4-diazepan-5-yl)methyl)-6-bromo-2-naphthamide
(106) N-(((3S,5S)-1-(2,2-diphenylethyl)-2-oxo-3-(3-(piperidin-1-yl)propyl)-1,4-diazepan-5-yl)methyl)-2-naphthamide
(107) N-(((3S,5S)-1-(2,2-diphenylethyl)-3-(3-guanidinopropyl)-2-oxo-1,4-diazepan-5-yl)methyl)-2-naphthamide
(108) N-(((3S,5S)-1-(2,2-diphenylethyl)-3-(3-guanidinopropyl)-2-oxo-1,4-diazepan-5-yl)methyl)biphenyl-4-carboxamide
(109) N-(((3S,5S)-1-(2,2-diphenylethyl)-3-(3-guanidinopropyl)-2-oxo-1,4-diazepan-5-yl)methyl)quinoline-3-carboxamide
(110) 1-(3-((5S,9aS)-7-benzhydryl-2-(biphenyl-4-ylmethyl)-3,6-dioxooctahydro-1H-imidazo[1,5-d][1,4]diazepin-5-yl)propyl)guanidine
(111) N-(((3S,5S)-1-(2,2-diphenylethyl)-3-(b3-guanidinopropyl)-2-oxo-1,4-diazepan-5-yl)methyl)-3-phenoxybenzamide
(112) N-(((3S,5S)-1-(2,2-diphenylethyl)-3-(3-guanidinopropyl)-2-oxo-1,4-diazepan-5-yl)methyl)-4-phenoxybenzamide
(113) N-(((3S,5S)-1-(2,2-diphenylethyl)-3-(3-guanidinopropyl)-2-oxo-1,4-diazepan-5-yl)methyl)-1H-indole-2-carboxamide
(114) 4-tert-butyl-N-(((3S,5S)-1-(2,2-diphenylethyl)-3-(3-guanidinopropyl)-2-oxo-1,4-diazepan-5-yl)methyl)benzamide
(115) N-(((3S,5S)-1-(2,2-diphenylethyl)-3-(3-guanidinopropyl)-2-oxo-1,4-diazepan-5-yl)methyl)-1-methoxy-2-naphthamide
(116) N-(((3S,5S)-1-(cyclohexylmethyl)-3-(3-guanidinopropyl)-2-oxo-1,4-diazepan-5-yl)methyl)-2-naphthamide
(117) N-(((3S,5S)-3-(3-(3,3-dimethylguanidino)propyl)-1-(2,2-diphenylethyl)-2-oxo-1,4-diazepan-5-yl)methyl)-2-naphthamide
(118) (S)—N—((S)-1-((3S,5S)-1-(2-(1H-indol-3-yl)ethyl)-3-(3-guanidinopropyl)-2-oxo-1,4-diazepan-5-yl)-2-phenylethyl)-2-acetamido-3-(1H-imidazol-4-yl)propanamide
(119) (S)—N—((R)-1-((3S,5S)-1-(2-(1H-indol-3-yl)ethyl)-3-(3-guanidinopropyl)-2-oxo-1,4-diazepan-5-yl)-2-phenylethyl)-2-acetamido-3-(1H-imidazol-4-yl)propanamide
(120) (E)-N-(((3S,5S)-3-(3-aminopropyl)-1-(2,2-diphenylethyl)-2-oxo-1,4-diazepan-5-yl)methyl)-3-p-tolylacrylamide
(121) (E)-N-(((3S,5S)-3-(3-aminopropyl)-1-(2,2-diphenylethyl)-2-oxo-1,4-diazepan-5-yl)methyl)-3-(4-fluorophenyl)acrylamide
(122) N-(((3S,5S)-3-(3-aminopropyl)-1-(2,2-diphenylethyl)-2-oxo-1,4-diazepan-5-yl)methyl)-6-fluoro-2-naphthamide
(123) N-(((3S,5S)-3-(3-(3-aminopropropyl)-1-(2,2-diphenylethyl)-2-oxo-1,4-diazepan-5-yl)methyl)-3,4-dichlorobenzamide
(124) N-(((3S,5S)-3-(3-(cyclohexylamino)propyl)-1-(2,2-diphenylethyl)-2-oxo-1,4-diazepan-5-yl)methyl)-2-naphthamide
(125) N-(((3S,5S)-3-(3-guanidinopropyl)-1-(naphthalen-2-yl)-2-oxo-1,4-diazepan-5-yl)methyl)-2-naphthamide
(126) N-(((3S,5S)-1-((9H-fluoren-9-yl)methyl)-3-(3-guanidinopropyl)-2-oxo-1,4-diazepan-5-yl)methyl)-2-naphthamide
(127) (E)-N-(((3S,5S)-3-(3-(cyclohexylamino)propyl)-1-(2,2-diphenylethyl)-2-oxo-1,4-diazepan-5-yl)methyl)-3-(4-fluorophenyl)acrylamide
(128) N-(((3S,5S)-3-(3-(3-aminopropropyl)-1-(2,2-diphenylethyl)-2-oxo-1,4-diazepan-5-yl)methyl)-5-(4-chlorophenyl)furan-2-carboxamide
(129) N-(((3S,5S)-3-(3-(3-aminopropropyl)-1-(2,2-diphenylethyl)-2-oxo-1,4-diazepan-5-yl)methyl)-5-(4-chlorophenyl)isoxazole-3-carboxamide
(130) N-(((3S,5S)-1-(2-cyclohexylethyl)-3-(3-guanidinopropyl)-2-oxo-1,4-diazepan-5-yl)methyl)-2-naphthamide
(131) N-(((3S,5S)-1-(2-(bicyclo[2.2.1]heptan-2-yl)ethyl)-3-(3-guanidinopropyl)-2-oxo-1,4-diazepan-5-yl)methyl)-2-naphthamide
(132) (E)-3-(4-chlorophenyl)-N-(((3S,5S)-1-(2,2-diphenylethyl)-2-oxo-3-(3-(piperidin-1-yl)propyl)-1,4-diazepan-5-yl)methyl)acrylamide
(133) (E)-N-(((3S,5S)-3-(aminomethyl)-1-(2,2-diphenylethyl)-2-oxo-1,4-diazepan-5-yl)methyl)-3-(4-chlorophenyl)acrylamide
(134) (E)-3-(4-chlorophenyl)-N-(((3S,5S)-1-(2,2-diphenylethyl)-2-oxo-3-(piperidin-1-ylmethyl)-1,4-diazepan-5-yl)methyl)acrylamide
(135) N-(((3S,5S)-3-(2-aminoethyl)-1-(2,2-diphenylethyl)-2-oxo-1,4-diazepan-5-yl)methyl)-3,4-dichlorobenzamide
(136) (E)-N-(((3S,5S)-3-(2-aminoethyl)-1-(2,2-diphenylethyl)-2-oxo-1,4-diazepan-5-yl)methyl)-3-(4-chlorophenyl)acrylamide
(137) 3,4-dichloro-N-(((3S,5S)-1-(2,2-diphenylethyl)-2-oxo-3-(2-(piperidin-1-yl)ethyl)-1,4-diazepan-5-yl)methyl)benzamide
(138) N-(((3S,5S)-3-(3-guanidinopropyl)-2-oxo-1-(2-phenoxy-2-phenylethyl)-1,4-diazepan-5-yl)methyl)-2-naphthamide
(139) N-(((3S,5S)-1-((3,5-dimethylcyclohexyl)methyl)-3-(3-guanidinopropyl)-2-oxo-1,4-diazepan-5-yl)methyl)-2-naphthamide
(140) N-(((3S,5S)-3-(2-aminoethyl)-1-(3,5-dichlorobenzyl)-2-oxo-1,4-diazepan-5-yl)methyl)-3,4-dichlorobenzamide
(141) (E)-N-(((3S,5S)-3-(2-aminoethyl)-1-(3,5-dichlorobenzyl)-2-oxo-1,4-diazepan-5-yl)methyl)-3-(4-chlorophenyl)acrylamide
(142) (S)-2-((5S,9aS)-2-(4-fluorobenzyl)-5-(3-guanidinopropyl)-3,6-dioxotetrahydro-1H-imidazo[1,5-d][1,4]diazepin-7(8H,9H,9aH)-yl)-N-methyl-3-(naphthalen-2-yl)propanamide
(143) (S)-2-((3S,5R)-5-((4-fluorobenzylamino)methyl)-3-(3-guanidinopropyl)-2-oxo-1,4-diazepan-1-yl)-N-methyl-3-(naphthalen-2-yl)propanamide
(144) N-(((3S,5S)-3-(3-aminopropyl)-2-oxo-1-((1-phenylcyclohexyl)methyl)-1,4-diazepan-5-yl)methyl)-2-naphthamide
(145) (E)-3-(4-chlorophenyl)-N-(((3S,5S)-1-(3,5-dichlorobenzyl)-2-oxo-3-(2-(piperidin-1-yl)ethyl)-1,4-diazepan-5-yl)methyl)acrylamide
(146) (E)-N-(((3S,5S)-3-(2-aminoethyl)-1-(2-ethylbutyl)-2-oxo-1,4-diazepan-5-yl)methyl)-3-(4-chlorophenyl)acrylamide
(147) N-(((3S,5S)-3-(3-guanidinopropyl)-2-oxo-1-(3-oxo-2-phenyl-3-(phenylamino)propyl)-1,4-diazepan-5-yl)methyl)-2-naphthamide
(148) N-(((3S,5S)-3-(aminomethyl)-1-(3,5-dichlorobenzyl)-2-oxo-1,4-diazepan-5-yl)methyl)-3,4-dichlorobenzamide
(149) N-(((3S,5S)-3-(aminomethyl)-1-(3,5-dichlorobenzyl)-2-oxo-1,4-diazepan-5-yl)methyl)-2-naphthamide
(150) (E)-N-(((3S,5S)-3-(aminomethyl)-1-(3,5-dichlorobenzyl)-2-oxo-1,4-diazepan-5-yl)methyl)-3-(4-chlorophenyl)acrylamide
(151) 3,4-dichloro-N-(((3S,5S)-1-(3,5-dichlorobenzyl)-2-oxo-3-(piperidin-1-ylmethyl)-1,4-diazepan-5-yl)methyl)benzamide
(152) (E)-3-(4-chlorophenyl)-N-(((3S,5S)-1-(3,5-dichlorobenzyl)-2-oxo-3-(piperidin-1-ylmethyl)-1,4-diazepan-5-yl)methyl)acrylamide
(153) (E)-N-(((3S,5S)-3-(2-aminoethyl)-2-oxo-1-(2-phenylbutyl)-1,4-diazepan-5-yl)methyl)-3-(4-chlorophenyl)acrylamide
(154) 6-chloro-N-(((3S,5S)-1-(2,2-diphenylethyl)-2-oxo-3-(2-(piperidin-1-yl)ethyl)-1,4-diazepan-5-yl)methyl)-2-naphthamide
(155) (E)-N-(((3S,5S)-1-(2,2-diphenylethyl)-2-oxo-3-(2-(piperidin-1-yl)ethyl)-1,4-diazepan-5-yl)methyl)-3-(4-isopropylphenyl)acrylamide
(156) (E)-N-(((3S,5S)-3-(2-aminoethyl)-1-(2,2-diphenylethyl)-2-oxo-1,4-diazepan-5-yl)methyl)-3-(4-isopropylphenyl)acrylamide
(157) (E)-N-(((3S,5S)-3-(2-aminoethyl)-1-(2,2-diphenylethyl)-2-oxo-1,4-diazepan-5-yl)methyl)-3-(2,4-dimethylphenyl)acrylamide
(158) (E)-3-(2,4-difluorophenyl)-N-(((3S,5S)-1-(2,2-diphenylethyl)-2-oxo-3-(2-(piperidin-1-yl)ethyl)-1,4-diazepan-5-yl)methyl)acrylamide
(159) (E)-3-(4-chlorophenyl)-N-(((3S,5S)-1-(2,2-diphenylethyl)-3-(2-morpholinoethyl)-2-oxo-1,4-diazepan-5-yl)methyl)acrylamide
(160) (E)-3-(4-chlorophenyl)-N-(((3S,5S)-3-(2-(2,5-dimethylpyrrolidin-1-yl)ethyl)-1-(2,2-diphenylethyl)-2-oxo-1,4-diazepan-5-yl)methyl)acrylamide
(161) (E)-3-(4-bromophenyl)-N-(((3S,5S)-1-(2,2-diphenylethyl)-2-oxo-3-(2-(piperidin-1-yl)ethyl)-1,4-diazepan-5-yl)methyl)acrylamide
(162) 5-(4-chlorophenyl)-N-(((3S,5S)-1-(2,2-diphenylethyl)-2-oxo-3-(2-(piperidin-1-yl)ethyl)-1,4-diazepan-5-yl)methyl)isoxazole-3-carboxamide
(163) 6-chloro-N-(((3S,5S)-2-oxo-1-((S)-2-phenylbutyl)-3-(3-(piperidin-1-yl)propyl)-1,4-diazepan-5-yl)methyl)-2-naphthamide
(164) (E)-N-(2-((3S,5S)-3-(2-aminoethyl)-1-(2,2-diphenylethyl)-2-oxo-1,4-diazepan-5-yl)propan-2-yl)-3-(4-chlorophenyl)acrylamide
(165) (E)-3-(4-chlorophenyl)-N-(2-((3S,5S)-1-(2,2-diphenylethyl)-2-oxo-3-(2-(piperidin-1-yl)ethyl)-1,4-diazepan-5-yl)propan-2-yl)acrylamide
(166) (S)-2-amino-3-(4-fluorophenyl)-N-(((3S,5R)-3-(3-guanidinopropyl)-1-((S)-1-(methylamino)-3-(naphthalen-2-yl)-1-oxopropan-2-yl)-2-oxo-1,4-diazepan-5-yl)methyl)propanamide
(167) (S)-2-((3S,5R)-5-((2-(4-chlorophenyl)acetamido)methyl)-3-(3-guanidinopropyl)-2-oxo-1,4-diazepan-1-yl)-N-methyl-3-(naphthalen-2-yl)propanamide
(168) (R)-2-amino-3-(4-fluorophenyl)-N-(((3S,5R)-3-(3-guanidinopropyl)-1-((S)-1-(methylamino)-3-(naphthalen-2-yl)-1-oxopropan-2-yl)-2-oxo-1,4-diazepan-5-yl)methyl)propanamide
(169) N-(((3R,5R)-3-(2-amino-2-methylpropyl)-2-oxo-1-((S)-2-phenylbutyl)-1,4-diazepan-5-yl)methyl)-6-chloro-2-naphthamide
(170) N-(((3S,5S)-3-(2-aminoethyl)-2-oxo-1-((S)-2-phenylbutyl)-1,4-diazepan-5-yl)methyl)-3,4-dichlorobenzamide diazepan-5-yl)methyl)-2-naphthamide
(171) 6-chloro-N-(((3S,5S)-3-(2-methyl-2-(piperidin-1-yl)propyl)-2-oxo-1-((S)-2-phenylbutyl)-1,4-diazepan-5-yl)methyl)-2-naphthamide
(172) 6-chloro-N-(((3R,5R)-3-(2-methyl-2-(piperidin-1-yl)propyl)-2-oxo-1-((S)-2-phenylbutyl)-1,4-diazepan-5-yl)methyl)-2-naphthamide
(173) (S)-2-amino-3-(4-chlorophenyl)-N-(((3S,5R)-3-(3-guanidinopropyl)-1-((S)-1-(methylamino)-3-(naphthalen-2-yl)-1-oxopropan-2-yl)-2-oxo-1,4-diazepan-5-yl)methyl)propanamide
(174) (R)-2-amino-3-(4-chlorophenyl)-N-(((3S,5R)-3-(3-guanidinopropyl)-1-((S)-1-(methylamino)-3-(naphthalen-2-yl)-1-oxopropan-2-yl)-2-oxo-1,4-diazepan-5-yl)methyl)propanamide
(175) (E)-3-(4-chlorophenyl)-N-(((3S,5R)-3-(3-guanidinopropyl)-1-((S)-1-(methylamino)-3-(naphthalen-2-yl)-1-oxopropan-2-yl)-2-oxo-1,4-diazepan-5-yl)methyl)acrylamide
(176) N-(((3R,5R)-1-(cyclohexylmethyl)-3-(3-guanidinopropyl)-2-oxo-1,4-diazepan-5-yl)methyl)biphenyl-4-carboxamide
(177) N-(((3S,5S)-2-oxo-1-((S)-2-phenylbutyl)-3-(2-(piperidin-1-yl)ethyl)-1,4-diazepan-5-yl)methyl)biphenyl-4-carboxamide
(178) N-(((3S,5S)-2-oxo-1-((S)-2-phenylbutyl)-3-(2-(piperidin-1-yl)ethyl)-1,4-diazepan-5-yl)methyl)-2-phenylthiazole-4-carboxamide
(179) 4′-chloro-N-(((3S,5S)-2-oxo-1-((S)-2-phenylbutyl)-3-(2-(piperidin-1-yl)ethyl)-1,4-diazepan-5-yl)methyl)biphenyl-2-carboxamide
(180) 6-chloro-N-(((3S,5S)-3-(2-(N-isopropylacetamido)ethyl)-2-oxo-1-((S)-2-phenylbutyl)-1,4-diazepan-5-yl)methyl)-2-naphthamide
(181) 6-chloro-N-(((3S,5S)-1-(2,2-diphenylethyl)-3-(2-morpholinoethyl)-2-oxo-1,4-diazepan-5-yl)methyl)-2-naphthamide
(182) 6-chloro-N-(((3R,5R)-2-oxo-1-((R)-2-phenylbutyl)-3-(2-(piperidin-1-yl)ethyl)-1,4-diazepan-5-yl)methyl)-2-naphthamide diazepan-5-yl)methyl)-2-naphthamide
(183) 6-chloro-N-(((3R,5R)-2-oxo-1-((S)-2-phenylbutyl)-3-(2-(piperidin-1-yl)ethyl)-1,4-diazepan-5-yl)methyl)-2-naphthamide
(184) N-(((3R,5S)-3-(4-aminobutyl)-2-oxo-1-phenethyl-1,4-diazepan-5-yl)methyl)biphenyl-4-carboxamide
(185) N-(((3S,5R)-1-benzhydryl-3-(3-guanidinopropyl)-2-oxo-1,4-diazepan-5-yl)methyl)biphenyl-4-carboxamide
(186) N-(((3R,5R)-1-benzhydryl-3-(3-guanidinopropyl)-2-oxo-1,4-diazepan-5-yl)methyl)biphenyl-4-carboxamide

As stated previously the compounds of formula (I) are modulators of the MC1R and therefore may be used to modulate the activity of MC1R or a fragment or analogue or functional equivalent thereof by exposing MC1R or a fragment or analogue or functional equivalent thereof to a compound of the invention. This can occur in vitro in assays where the modulation of MC1R activity is desirable, however it is typically more beneficial when utilised in modulation of MC1R activity in a patient. The amount of modulation provided by the compounds of the invention will vary from compound to compound and will also be affected by the amount of compound administered. The modulation can consist of upregulation or downregulation. In one embodiment the amount of upregulation or downregulation is at least 10%. In another embodiment the amount of upregulation or downregulation is at least 20%. In an even further embodiment the amount of upregulation or downregulation is at least 50%.

Accordingly the methods of the present invention may be used in the treatment of any condition in which modulation of the activity of MC1R or a fragment or analogue or functional equivalent thereof would lead to a beneficial effect on that condition. As such the compounds suitable for use in the present invention may be used in methods of preventing or treating a condition associated either directly or indirectly with the activity of MC1R or a fragment or analogue or functional equivalent thereof in a mammal wherein an MC1R modulating amount of the compound of the invention is administered to the mammal. One condition associated with MC1R activity is pigmentation and conditions related thereto. In one embodiment of the method the condition is selected from the group consisting of hyperpigmentation (including melasma), hypopigmentation (including vitiligo), melanoma, basal cell carcinoma, squamous cell carcinoma, erythropoietic protoporphyria, polymorphous light eruption, solar urticaria, photosensitivity, and sunburn.,

For example, down regulation of MC1R leads to a reduction in pigmentation and can thus be used in the treatment or prophylaxis of a number of conditions in which reduced pigmentation is desirable, such as vitiligo or melasma. Decreased pigmentation may also be desirable for a purely cosmetic effect.

In another example, upregulation of MC1R leads to an increase in pigmentation and can thus be used in the treatment or prophylaxis of a number of conditions in which increased pigmentation is desirable, such as vitiligo, melasma, melanoma, basal cell carcinoma, squamous cell carcinoma, erythropoietic protoporphyria, polymorphous light eruption, solar urticaria, photosensitivity or sunburn. Increased pigmentation may also be desirable for a purely cosmetic effect.

The methods of the invention may also be useful in the prevention or treatment of a number of conditions that relate to biological processes controlled by MC1R, such as diseases related to inflammation, aberrant fibroblast activity and pain. The compounds of formula (I) may also be useful for the treatment or prevention of cancers, such as melanoma, basal cell carcinoma, and squamous cell carcinoma, that involve MC1R-associated biological processes not directly related to pigmentation.

Due to their impact on pigmentation the compounds of formula (I) may also find application in treatments where altered pigmentation is desirable such as in cosmetic treatments. The compounds may thus be used in methods of increasing or reducing pigmentation in a mammal, the method comprising administering an effective amount of a compound of formula (I).

The compounds of formula (I) may be used in the treatment of conditions in any species in which MC1R is present, most typically mammals. Examples of species in which MC1R is found (and hence species in which the compounds may be used) include humans, rats, mice, dogs, and rhesus monkey. In a specific embodiment the mammal is a human.

As discussed above the compounds of formula (I) are also useful as they bind to MC1R and this binding ability may be utilised in either therapeutic or in diagnostic applications. In each instance both therapy and diagnosis will rely on the compound of formula (I) binding to or localising in the desired tissues or organs containing the MC1R of the subject being treated/diagnosed.

The binding of the compounds of formula (I) to MC1R may therefore be utilised to take advantage of the binding properties. For example the binding may be used in methods of diagnosis or monitoring of a medical condition. In this way the methods typically utilise methods of detection of the extent of binding by determining the amount of compound of formula (I) present or the amount of the label attached to the compound of formula (I).

The detection of the compound of formula (I) may occur either in vitro or in vivo. If it is carried out in vivo it typically involves an imaging technique.

Thus for example in methods of diagnosis they would typically involve administration of an amount of the compound of formula (I) or a labelled form thereof to a subject followed by monitoring of the subject after a suitable time period to determine if the compound has localised at a particular location in the body or whether the compound is broadly speaking evenly distributed through the body. As a general rule where the compound is localised in tissue or an organ of the body this is indicative of the presence in that tissue or organ of the MC1R receptor.

The monitoring of the subject for the location of the compound of formula (I) or a labelled form thereof will typically provide the analyst with information regarding the location of the compound of formula (I) and hence the location of any material that contains appreciable amounts of MC1R. The clinician can then compare the determined amount of compound of formula (I) with the expected reading to determine whether there is an elevated expression of MC1R in the location and hence the probability of the person having an MC1R related condition. Accordingly, diagnosis of a disease according to the present invention can be effected by determining a level of the amount of MC1R in a location in the subject (if in vivo) or the level in a biological sample obtained from the subject, wherein the level determined can be correlated with predisposition to, or presence or absence of the disease.

If the diagnosis is based on a biological sample obtained from a subject this can be any form of biological sample known in the art. For example the sample may be blood, saliva, cerebrospinal fluid or a tissue sample. Examples of tissue samples include, but are not limited to, fine needle biopsy, needle biopsy, core needle biopsy and surgical biopsy (e.g., brain biopsy), and lavage.

Irrespective of whether the detection occurs in vivo or in vitro the determined level of MC1R in the sample is then compared with the known background or expected level to determine whether there is an increase in expression of MC1R in the patient. Any observable difference is then correlated with the probability that the patient has the condition.

The methods of the present invention may also be used in methods of monitoring the progress of a condition which leads to increased levels of MC1R expression. In these methods the steps as discussed above are broadly speaking the same with the difference being that after the initial reading of a patient at each subsequent test the level of MC1R activity is compared with the level at the previous test rather than with an expected baseline. In this way the progression of the disease in the patient may be monitored.

These methods typically involve the binding a compound of formula (I) or a labelled form thereof to MC1R or a fragment, analogue or functional equivalent thereof and analysing the material to determine the extent of the binding typically by detecting the presence of the compound of formula (I) or labelled form thereof.

The binding of the compounds of formula (I) may also be used in therapeutic applications in which the compounds are used in methods of delivering an active agent to the MC1R or a fragment, analogue or functional equivalent thereof in a mammal. Thus for example the compound of formula (I) may have an active agent attached to it which can be delivered by the compound of formula (I) to the receptor. In this way the compound of formula (I) is in effect acting as a vector for the active agent.

The active agent that is delivered by taking advantage of the binding behaviour may be any suitable active agent that has activity at the site of interest. Thus for example it may be an active agent that has biological activity per se at the receptor site leading to an improved therapeutic effect directly. For example, the active agent may be a radionuclide that is concentrated at the targeted site, resulting in the desired therapeutic effect. Examples of radionuclides of this type are well known in the art as are the methods of treating subjects with them. Alternatively the active agent may be one that has to be “activated” at the site before its activity becomes apparent. For example the active agent may be one that only becomes active when the active agent is cleaved or released from the compound of formula (I). The active agent may also be a radionuclide that is activated by exposing the patient or subject to irradiation at the appropriate wavelength and intensity leading to the radionuclide having the desired therapeutic effect. Examples of radionuclides of this type are well known in the art as are the methods of treating subjects with them.

In addition the treatment regime may involve a single administration or multiple administrations. In respect of radiotherapy applications these will typically involve a number of cycles of radiation treatment with the cycles being continued until such time as the condition has been ameliorated. Once again the optimal number of cycles and the spacing between each treatment cycle will depend upon a number of factors such as the severity of the condition being treated, the health (or lack thereof) of the subject being treated and their reaction to radiotherapy. In general the optimal dosage amount and the optimal treatment regime can be readily determined by a skilled addressee in the art using well known techniques.

Administration of compounds within Formula (I) to a patient such as humans can be by topical administration, by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier or diluent and in an amount sufficient to deliver to the patient a therapeutically effective dose.

In using the compounds of formula (I) they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. We refer the reader to Remingtons Pharmaceutical Sciences, 19^{th} edition, Mack Publishing Co. (1995) for further information.

The compounds of formula (I) can be administered alone or in the form of a pharmaceutical composition in combination with a pharmaceutically acceptable carrier, diluent or excipient. The compounds of formula (I), while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. The compositions are prepared in manners well known in the art.

A compound of formula (I) is typically combined with the carrier to produce a dosage form suitable for the particular patient being treated and the particular mode of administration. For example, a formulation intended for the oral administration to humans may contain from about 0.5 mg to about 5 g of the compound of the invention, compounded with an appropriate and convenient amount of carrier material which may vary from about 5 to about 99.95 percent of the total composition. Representative dosage forms will generally contain between from about 1 mg to about 500 mg of a compound of the invention, typically 25 mg, 50 mg, 100 mg, 200 mg, 300 mg, 400 mg, 500 mg, 600 mg, 800 mg, or 1000 mg. Compounds of the present invention may also be formulated for topical delivery in formulations such as solutions, ointments, lotions, gels, creams, microemulsions or transdermal patches. For example, these topical formulations may contain from 0.005 to 5% (wt/wt or wt/vol) of a compound of the invention.

The compounds of formula (I) may be used or administered in combination with one or more additional drug (s), either concurrently or sequentially. The compounds of the present invention may be used in combination with one or more other pharmaceutically-active compounds, such as other pigmentation altering, anticancer, anti-inflammatory, or pain medications. These components can be administered in the same formulation or in separate formulations. If administered in separate formulations the compounds of the invention may be administered sequentially or simultaneously with the other drug(s).

Pharmaceutical compositions suitable for use in the invention for parenteral injection comprise pharmaceutically acceptable sterile aqueous or non aqueous solutions, dispersions, suspensions or emulsions as well as sterile powders for reconstitution into sterile injectable solutions or dispersions just prior to use. Examples of suitable aqueous and non aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

For topical administration, the active agent may be in the form of an ointment, cream, suspension, lotion, powder, solution, paste, gel, spray, aerosol or oil. Alternatively, the composition may be delivered via a liposome, nanosome, rivosome, or nutri-diffuser vehicle. Alternately, a formulation may comprise a transdermal patch or dressing such as a bandage impregnated with an active ingredient and optionally one or more carriers or diluents. To be administered in the form of a transdermal delivery system, the dosage administration will, of course, be continuous rather than intermittent throughout the dosage regimen. Methods for producing formulations for topical administration are known in the art.

The compositions used for topical administration typically contain a pharmaceutically acceptable carrier which may be any vehicle that is toxicologically and pharmaceutically acceptable. Typical pharmaceutically acceptable carriers that can be used in compositions of the present invention include water, ethanol, acetone, isopropyl alcohol, stearyl alcohol, freons, polyvinyl pyrrolidone, propylene glycol, polyethlyene glycol, fragrances, gel-producing materials, mineral oil, stearic acid, spermaceti, sorbitan, monoleate, polysorbates, “Tweens,” sorbitol, methyl cellulose, petrolatum, a mineral oil (vaseline oil), which may be any petroleum based product; modified or unmodified vegetable oils such as peanut oil, wheatgerm oil, linseed oil, jojoba oil, apricot kernel oil, walnut oil, palm oil, pistachio oil, sesame oil, colza oil, cade oil, corn germ oil, peach kernel oil, poppyseed oil, pine oil, castor oil, soya oil, safflower oil, coconut oil, hazelnut oil, grapeseed oil, avocado oil, soy oil, sweet almond oil, calophyllum oil, castor oil, olive oil, sunflower oil, or animal oils such as whale oil, seal oil, menhaden oil, halibut liver oil, cod liver oil, cod, tuna, turtle tallow, horse's hoof, sheep's foot, mink, otter, marmot oil and the like; synthetic oils such as silicon oil such as dimethylpolysiloxane; alkyl and alkenyl esters of fatty acids, such as isopropyl esters of myristic, palmitic and stearic acids and fatty esters which are solid at room temperature; waxes such as lanolin wax, candelilla wax, spermaceti, cocoa butter, karite butter, silicon waxes, hydrogenated oils which are solid at room temperature, sucro-glycerides, oleates, myristates, linoleates, stearates, paraffin, beeswax, carnauba wax, ozokerite, candelilla wax, microcrystalline wax; fatty alcohols such as lauryl, cetyl, myristyl, stearyl, palmityl and oleyl alcohols; polyoxyethylated fatty alcohols; and wax esters, lanolin and its derivatives, perhydrosqualene and saturated esters, ethyl palmitate, isopropyl palmitate, alkyl myristates such as isopropyl myristate, butyl myristate and decyl myristate, hexyl stearate, triglyceride esters, triglycerides of octanoic and decanoic acid, cetyl ricinoleate, stearyl octanoate (Purcellin oil), fatty acids, polyhydric alcohols, polyether derivatives, fatty acid monoglycerides, polyethylene gylcol, propylene glycol, alkyl ethoxy ether sulfonates, ammonium alkyl sulfates, fatty acid soaps, and hydrogenated polyisobutene, and mixtures of waxes and oils.

The compositions for topical administration may be formulated in numerous forms. However, the composition may often take the form of an aqueous or oily solution or dispersion or emulsion or a gel or a cream. An emulsion may be an oil-in-water emulsion or a water-in-oil emulsion.

The oil phase of water-in-oil or oil-in-water emulsions may comprise for example: a) hydrocarbon oils such as paraffin or mineral oils; b) waxes such as beeswax or paraffin wax; c) natural oils such as sunflower oil, apricot kernel oil, shea butter or jojoba oil; d) silicone oils such as dimethicone, cyclomethicone or cetyldimethicone; e) fatty acid esters such as isopropyl palmitate, isopropyl myristate, dioctylmaleate, glyceryl oleate and cetostearyl isononanoate; f) fatty alcohols such as cetyl alcohol or stearyl alcohol and mixtures thereof (eg cetearyl alcohol); g) polypropylene glycol or polyethylene glycol ethers, eg PPG-14 butyl ether; or h) mixtures thereof.

Emulsifiers used may be any emulsifiers known in the art for use in water-in-oil or oil-in-water emulsions. Known cosmetically acceptable emulsifiers include: a) sesquioleates such as sorbitan sesquioleate, available commercially for example under the trade name Arlacel 83 (ICI), or polyglyceryl-2-sesquioleate; b) ethoxylated esters of derivatives of natural oils such as the polyethoxylated ester of hydrogenated castor oil available commercially for example under the trade name Arlacel 989 (ICI); c) silicone emulsifiers such as silicone polyols available commercially for example under the trade name ABIL WS08 (Th. Goldschmidt AG); d) anionic emulsifiers such as fatty acid soaps e.g. potassium stearate and fatty acid sulphates e.g. sodium cetostearyl sulphate available commercially under the trade name Dehydag (Henkel); e) ethoxylated fatty alcohols, for example the emulsifiers available commercially under the trade name Brij (ICI); f) sorbitan esters, for example the emulsifiers available commercially under the trade name Span (ICI); g) ethoxylated sorbitan esters, for example the emulsifiers available commercially under the trade name Tween (ICI); h) ethoxylated fatty acid esters such as ethoxylated stearates, for example the emulsifiers available commercially under the trade name Myrj (ICI); i) ethoxylated mono-, di-, and tri-glycerides, for example the emulsifiers available commercially under the trade name Labrafil (Alfa Chem.); j) non-ionic self-emulsifying waxes, for example the wax available commercially under the trade name Polawax (Croda); k) ethoxylated fatty acids, for example, the emulsifiers available commercially under the trade name Tefose (Alfa Chem.); 1) methylglucose esters such as polyglycerol-3 methyl glucose distearate available commercially under the name Tegocare 450 (Degussa Goldschmidt); or m) mixtures thereof.

Gels for topical administration may be aqueous or non-aqueous. Aqueous gels are preferred. The gel will contain a thickening agent or gelling agent in order to give sufficient viscosity to the gel. A variety of thickening agents may be used according to the nature of the liquid carrier and the viscosity required and these are recited hereinafter. A particularly suitable thickener is a copolymer of acryloyl dimethyl tauric acid (or a salt thereof), preferably a copolymer of that monomer with another vinylic monomer. For example, the thickening agent is a copolymer of a salt of acryloyl dimethyl tauric acid with another vinylic monomer. The salt may be a salt of a Group I alkali metal, but is more preferably an ammonium salt. Examples of suitable copolymer thickening agents are: i) Ammonium acryloyl dimethyl taurate I vinyl pyrrolidone copolymer, ie a copolymer of ammonium acryloyl dimethyl taurate and vinyl pyrrolidone (1-vinyl-2-pyrrolidone).

The composition may additionally comprise other skincare active agents which are well known in the art which may be effective to aid the normal functioning of the skin. One group of preferred compositions comprise hydrolysed milk protein to regulate sebum production.

The composition may additionally comprise other components which will be well known to those skilled in the art such as emollients, humectants, emulsion stabilising salts, preservatives, chelating agents or sequestering agents (sequestrants), abrasives, anti-oxidants, stabilisers, pH adjusters, surfactants, thickeners, diluents, perfumes and colourings.

The topical formulations may desirably include a compound that enhances absorption or penetration of the active ingredient through the skin or other affected areas. Examples of such dermal penetration enhancers include dimethylsulfoxide and related analogues.

The amount of compound administered will preferably treat and reduce or alleviate the condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, sex, diet, the specific condition involved, the severity of the condition, the response of the patient to treatment, the particular compound administered, the mode of administration, the bioavailability of the preparation administered, the dose regime selected, the use of other medications and other relevant circumstances.

A preferred dosage will be a range from about 0.01 to 300 mg per kilogram of body weight per day. A more preferred dosage will be in the range from 0.1 to 100 mg per kilogram of body weight per day, more preferably from 0.2 to 80 mg per kilogram of body weight per day, even more preferably 0.2 to 50 mg per kilogram of body weight per day. A suitable dose can be administered in multiple sub-doses per day.

Synthesis of Compounds for Use in the Invention

The general synthetic route to the compounds for use in the invention proceeds through the key intermediate A, produced as outlined in Schemes 1 or 2.

In Scheme 1, an amino acid derivative V—N(R²)—Y—CO₂H (V═R¹X or an amine protecting group P¹) is converted to a Weinreb amide via activation of the carboxyl group and amidation with N-methyl methoxyamine. Addition of a vinyl Grignard reagent produces the aminoalkyl vinyl ketone, which undergoes conjugate addition by the R⁶—(CR^{5a}R^{6b})ᵣNH₂ amine component (shown as WNH₂ for simplicity). The resulting secondary amine is acylated under standard peptide coupling conditions with the protected amino acid, P²—NHCH(U)—CO₂H, where U represents either the final R side chain, a protected final side chain R—P³, or a precursor that requires chemical modification to form the final R side chain. Deprotection of the P² protecting group is followed by intramolecular reductive amination of the ketone using standard reduction conditions, such as H₂/Pd catalyst, NaBH₄, NaBH₃CN, or NaBH(OAc)₃, forming key intermediate A. If Y═CH₂ or CH₂CH₂, A is formed as the predominant diastereomer. If V═R¹X and U═R, A is the final product.

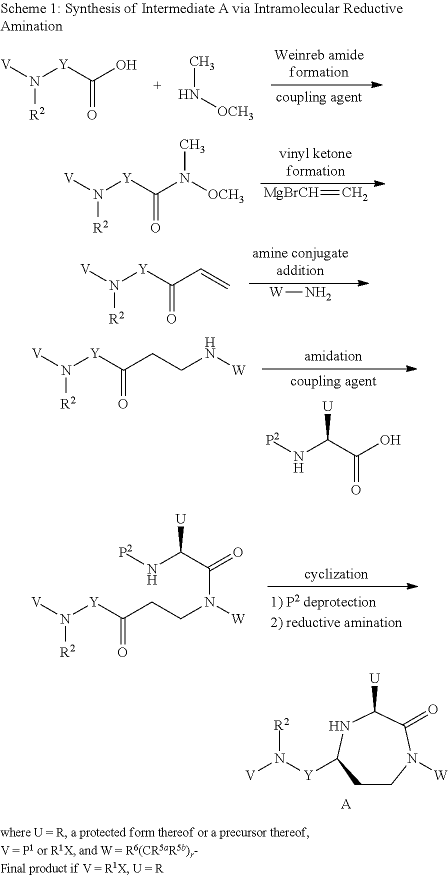

In Scheme 2, an alternate route to the desired intermediate A begins with the same Weinreb amide formation, vinyl Grignard addition, and amine conjugate addition. At this point, the secondary amine is protected with an amine protecting group P⁴. The ketone is then reductively aminated with a protected amino ester, H₂NCH(U)—CO₂P5, producing a mixture of diastereomers that are carried through the next reaction steps. The ring system is generated by deprotection of the P⁴ and P⁵ protecting groups, followed by amide bond formation using standard peptide coupling reagents. Alternatively, the P⁴ protecting group is removed and cyclization achieved by thermal or base-induced cyclization with the P⁵-protected ester. The cyclization produces a mixture of two diastereomers, A and B, from which the preferred diastereomers A can be separated by chromatography.

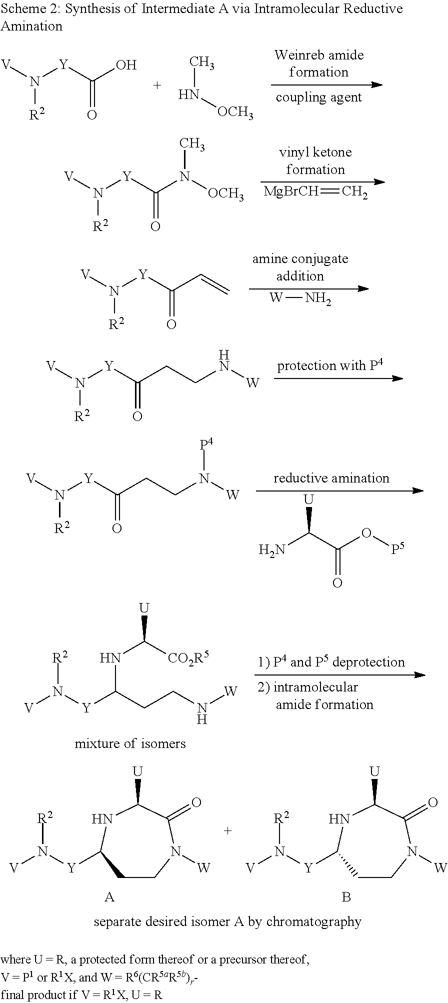

The key intermediate A may be the final product if U═R and V═R¹X, but otherwise is converted into the final product as illustrated in Schemes 3, 4 and 5.

In Scheme 3, where V═R¹X, the final product is obtained by modification of the U side chain, such as removal of a P³ protecting group, or removal of a P³ protecting group followed by further chemical modification.

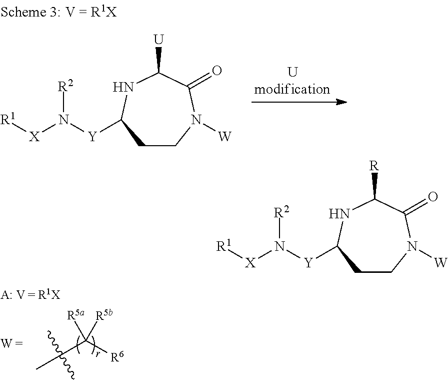

In Scheme 4, where V═P¹, the final product is obtained by removal of the P1 protecting group followed by introduction of the R¹X substituent. If U═R, this produces the final product. Alternatively, the U side chain is then modified to produce the final R group as in Scheme 3.

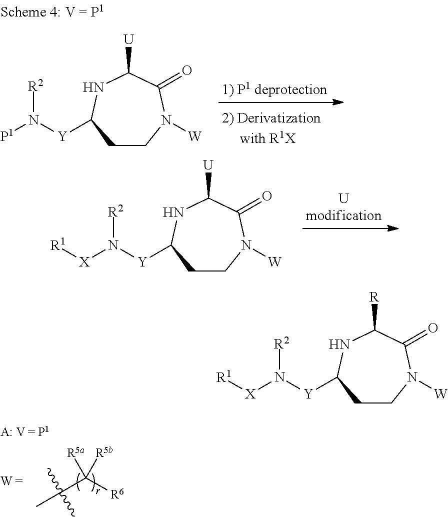

In Scheme 5, where V═P¹, the final product is obtained by first modifying the U side chain to produce the final R group as in Scheme 3. This is followed by removal of the P1 protecting group followed by introduction of the R¹X substituent.

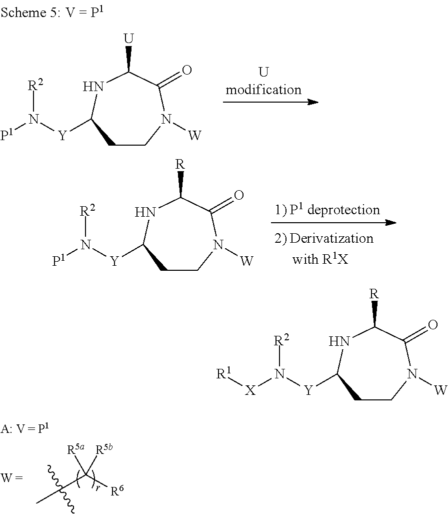

It is also possible to modify the W substituent, if desired, during these reaction sequences.

### EXAMPLES

The following examples are intended to illustrate the embodiments disclosed and are not to be construed as being limitations thereto. Additional compounds, other than those described below, may be prepared using the following described reaction schemes as discussed above or appropriate variations or modifications thereof. All starting materials described in the Examples below are commercially available or readily synthesized by those skilled in the art.

### Instrumentation

HPLC analyses were carried out on an Agilent 1100 Series Purification

System with a Phenomenex Synergi 4μ Max-RP 80A, 50×2.00 mm analytical HPLC column, with peak detection by UV. The standard analysis employed a 1 mL/min flow rate of 0.05% trifluoroacetic acid (TFA) in water (Solvent A) and 0.05% TFA in 90:10 acetonitrile:water (Solvent B), using a gradient of 5% B (initial) to 95% B over 9 min. Mass spectra were run on an Applied Biosystems MDS Sciex API 2000 LC/MS/MS triple quadrupole mass spectrometer and analyzed by ion spray mass spectrometry (ISMS). Preparative scale HPLC was carried out on a Waters Delta Prep 3000 HPLC system with peak detection by UV (Waters model 486 tunable absorbance detector), using Phenomenex Luna 10μ C5 100A, 250×21.20 mm (20 mg scale), Phenomenex Luna 15μ C8(2) 100A, 250×30.00 mm (50 mg scale), or Phenomenex Luna 15μ C8(2) 100A, 250×50.00 mm (100 mg scale) HPLC columns. The solvent system employed various gradients of 0.05% TFA in water (Solvent A) and 0.05% TFA in 90:10 acetonitrile:water (Solvent B).

The following examples 1 to 7 provide general synthetic procedures that may be followed in order to carry out the transformations described in schemes 1 to 5. In order to make different end products using these procedures it is necessary to either vary a variable group on the starting material or to vary a variable group on one of the reagents depending upon the nature of the reaction. It will be apparent to a skilled addressee from a reading of the general procedures how to vary either the starting material or the reagents used in the procedure to produce differing end products. In addition depending upon the starting materials and the reagents it may be necessary and/or desirable to make slight modifications to the described general procedures in order to provide the most facile synthesis of the desired end product.

### Example 1

### General Procedure—Weinreb Amide Formation

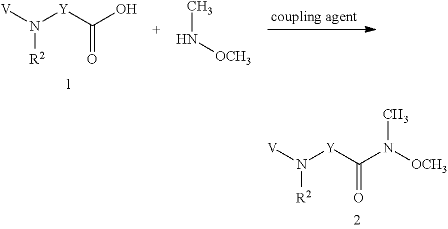

BOP reagent (100 mmol) and diisopropylethylamine (DIPEA) (100 mmol) is added to a stirred solution of the amino acid (1) (100 mmol) in dichloromethane (DCM) (100 mL). The solution is then stirred at room temperature for 10 mins, before addition of a premixed solution of N,O-dimethylhydroxylamine hydrochloride (100 mmol) and DIPEA (100 mmol) followed by stirring at room temperature overnight. The DCM is then removed by rotary evaporation and the residue taken up in ethyl acetate (EtOAc) (200 mL). The organic phase is then washed with 1N HCl (3×100 mL), H₂O (3×100 mL), saturated NaHCO₃ aqueous solution (3×100 mL) and brine (1×10 mL). The organic phase is then dried (MgSO₄) and the EtOAc removed to give the Weinreb amide (2) as a white solid or an oil.

### Example 2

### General Procedure—Vinyl Grignard Addition to Weinreb Amide to Form α,β-unsaturated Ketones of Formula (3)

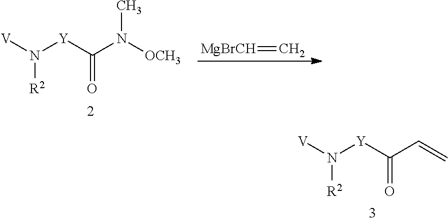

To the Weinreb amide (2) (15 mmol) in DCM (10 mL) at 0° C. is added vinyl magnesium bromide (45 mmol) in THF (45 mL). The reaction is stirred for 2 hrs and monitored by HPLC. The reaction is then quenched by adding it to a mixture of ice and 1M HCl (200 mL). The aqueous mixture is extracted with DCM (3×100 mL) and the organic layers combined and washed with 1M HCl (2×200 mL) and H₂O (3×100 mL). The organic phase is dried (MgSO₄) to provide a solution of the α,β-unsaturated ketone (3). The α,β-unsaturated ketone (3) may be isolated by rotary evaporation or it may be used in solution without further purification. If the intention is to use the α,β-unsaturated ketone (3) in solution the volume is reduced to 100 mL by rotary evaporation and stored for later use.

### Example 3

### General Procedure—Conjugate Addition of Amine to α,β-Unsaturated Ketones of Formula (3) to Produce Compounds of Formula (4) Amine Conjugate

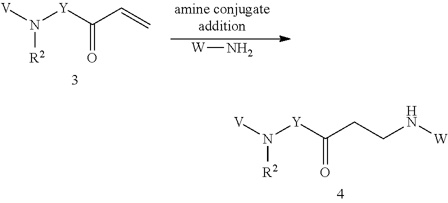

To the amine W—NH₂ (7.4 mmol) in DCM (10 mL) is added a solution of the α,β-unsaturated ketone (3) (5.7 mmol) in DCM (50 mL). The solution is stirred at room temperature for 15 mins, or until analysis indicates that all of (3) has been consumed. The solution of compound (4) is immediately used without purification for the subsequent reaction.

### Example 4

### General Procedure—Acylation of Aminoketone (4)

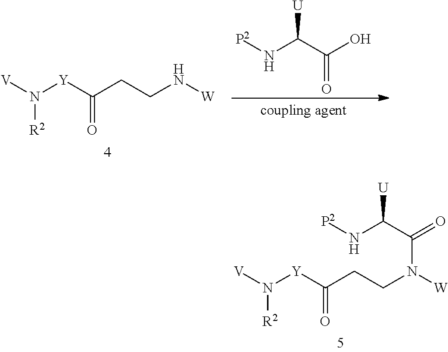

The amine acid P²—NHCH(U)—CO₂H (15 mmol) and DIC (15 mmol) is added to a solution of DCM containing 10 mmol of the conjugate addition adduct 4. The reaction is stirred at room temperature overnight. The DCM is removed by rotary evaporation and the residue is then subjected to column chromatography on silica gel using petroleum spirit:EtOAc to give 5.

As an alternative, the DIC may be replaced with HATU (15 mmol) and DIPEA (15 mmol). The reaction is stirred at room temperature overnight. The DCM is removed by rotary evaporation and the residue is taken up in EtOAc (100 mL). The organic layer is washed with saturated sodium bicarbonate solution (2×100 mL), saturated ammonium chloride solution (2×100 mL) and brine (2×100 mL). The organic phase is dried and the solvent removed under reduced pressure. The residue is subjected to column chromatography on silica gel using petroleum ether:EtOAc to give 5.

### Example 5

### General Procedure—P² Deprotection and Cyclization

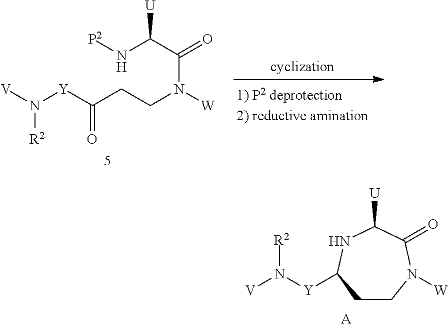

The procedure adopted for the removal of the P2 protecting group will vary depending upon the exact nature of the protecting group. As will be appreciated by a skilled addressee a large number of possible protecting groups may be used and a skilled worker in the art will readily be able to determine an appropriate procedure for the removal of any particular protecting group from procedures known in the art. Nevertheless in order to assist the reader general procedures for the removal of the more common protecting groups are provided.

P²=Fmoc: To compound 5 (2 mmol) in DCM (3 mL) is added diethylamine (20 mmol). The reaction is stirred at room temperature for 1 hr. The DCM and diethylamine is then removed by rotary evaporation. DCM (5 mL) and sodium triacetoxyborohydride (3 mmol) are then added, and the reaction stirred overnight at room temperature. The organic phase is washed with saturated sodium bicarbonate solution (25 mL), dried (MgSO₄) and the DCM removed to give the cyclised product A. This may be purified by flash chromatography on silica gel or used without purification.

P²=Boc: To compound 5 (2 mmol) in DCM (3 mL) is added TFA (3 mL) and the reaction stirred at room temperature for 2 hrs. The DCM and TFA are then removed by rotary evaporation. DCM (5 mL) and sodium triacetoxyborohydride (3 mmol) is then added, and the reaction stirred overnight at room temperature. The organic phase is washed with saturated sodium bicarbonate solution (25 mL), dried (MgSO₄) and the DCM removed to give the cyclised product A. This may be purified by flash chromatography on silica gel or used without purification.

P²=Cbz: A mixture of crude 5 (1 mmol) and 5% Pd/C (200 mg) in 2-propanol (15 mL) is shaken at room temperature under hydrogen (30 psi) for 24 hrs. The mixture is then filtered through a pad of Celite and the filtrate concentrated under reduced pressure to give a crude product. Purification by flash chromatography on silica gel (100% EtOAc) may be used to give A.

### Example 6

### General Procedure—P¹ Deprotection and Derivatization with R¹X

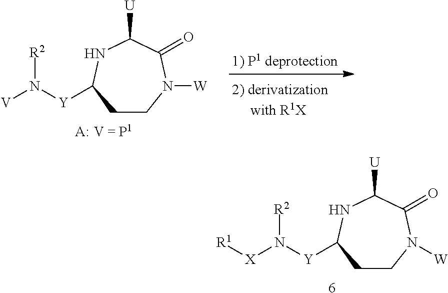

The procedure adopted for the removal of the P1 protecting group will vary depending upon the exact nature of the protecting group. As will be appreciated by a skilled addressee a large number of possible protecting groups may be used and a skilled worker in the art will readily be able to determine an appropriate procedure for the removal of any particular protecting group from procedures known in the art. Nevertheless in order to assist the reader general procedures for the removal of the more common protecting groups are provided.

Deprotection, P¹=Cbz: To the cyclised product A (1 mmol) in methanol (5 mL) is added catalytic Pd/C. The reaction is stirred under a hydrogen atmosphere overnight. The reaction mixture is filtered through Celite and the methanol removed by rotary evaporation to give the free amine. The amine may be used in the next reaction without purification.

Deprotection, P¹=Boc: To the cyclised product A (1 mmol) in DCM (1 mL) is added TFA (1 mL) and the reaction stirred at room temperature for 2 hrs. The solvent is removed by rotary evaporation to give the amine TFA salt, which may be used in the next reaction without purification.

Deprotection, P¹=Alloc: To the cyclised product A (1 mmol) in DCM (6 mL) is added 1,3-dimethylbarbituric acid (0.2 mmol) and palladium tetrakis triphenylphosphine (10 mg). The reaction is evacuated and stirred at room temperature for 1 hr. The DCM is removed under reduced pressure to give the crude free amine, which may be used in the next reaction without purification.

Derivatisation with R¹X when X═C(═O): To the free amine (1 mmol) in DCM (5 mL) is added DIPEA (1 mmol.), BOP reagent (1.5 mmol) and acid component R¹CO₂H (1.5 mmol). The reaction is stirred at room temperature for 2 hrs. Rotary evaporation and preparative HPLC gives the purified adduct.

### Example 7

### General Procedure—U Modification via P³ Deprotection and Dialkylation with Dibromide

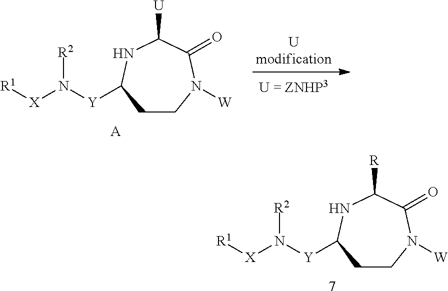

The procedure adopted for modification of U via deprotection and derivatization will vary depending on the exact nature of the U group. As will be appreciated by a skilled addressee a large number of modifications are possible, and a skilled worker in the art will readily be able to determine an appropriate procedure for the conversion into a desired R group. Nevertheless in order to assist the reader, one general modifcation procedure commonly employed for a number of the following examples is provided.

P³=Boc: To the protected amine (1 mmol) in DCM (5 mL) is added TFA (5 mL) and the reaction stirred at room temperature for 2 hrs. DCM (20 mL) is added and the solution is washed with saturated sodium bicarbonate solution (20 mL), dried (MgSO₄) and evaporated to give the crude amine. To the crude amine is added DMF (0.5 mL), potassium carbonate (50 mg) and 1,5-dibromopentane (5 mmol). The reaction mixture is stirred at room temperature for 1.5 hrs, after which DCM (20 mL) is added, the organic layer washed with saturated sodium bicarbonate solution (20 mL) and H₂O (20 mL), dried (MgSO₄) and evaporated. The residue may be purified by preparative HPLC to give the piperidinyl product. The purified product is isolated as the TFA salt, but is readily converted into the free base via neutralisation with aqueous NaHCO₃ and extraction into an organic solvent, or further converted into the HCl salt by acidification with 1N HCl.

### Example 8

### Synthesis of Compound 8 N-(2-(methoxy(methyl)amino)-2-oxoethyl)-2-naphthamide

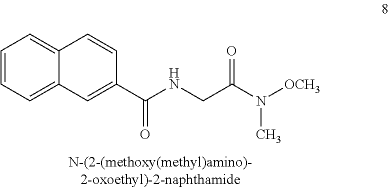 [SMILES: CON(C)C(=O)CNC(=O)c1ccc2ccccc2c1]

To a mixture of 2-naphthoic acid (5.8 g, 33.7 mmol), 2-amino-N-methoxy-N-methylacetamide (Gly Weinreb amide; prepared from Boc-Gly Weinreb amide 49 following the alternative procedure of Example 26) (3.8 g, 32.1 mmol) and DIPEA (12.0 mL, 68.9 mmol) in DCM (70 mL) was added BOP (14.9 g, 33.7 mmol) in one portion at room temperature. The resulting mixture was stirred for 1 hr then saturated NaHCO₃ aqueous solution was added. The organic layer was washed with brine (5×60 mL) and 1 N HCl (2×30 mL), dried over MgSO₄, filtered and concentrated under reduced pressure to give the crude product, which was used in the next reaction without further purification.

### Example 9

### Synthesis of Compound 9 N-(2-(methoxy(methyl)amino)-2-oxoethyl)-2-naphthamide

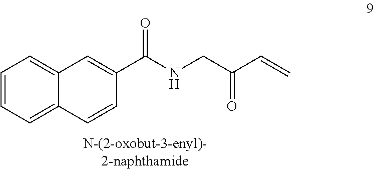 [SMILES: C=CC(=O)CNC(=O)c1ccc2ccccc2c1]

To a solution of 8 (3.5 g, 12.85 mmol) in dry THF (10 mL) was added a solution of vinylmagnesium bromide in THF (1 M, 31 mL) slowly at 0° C. After addition, the resulting mixture was stirred at room temperature for 1 hr then was poured into an icy 1 N HCl solution (50 mL). The aqueous layer was extracted with DCM (3×80 mL) and the combined organic layers were dried over MgSO₄, filtered and concentrated under reduced pressure to give the crude product. MS (ESI) 240 (M+1); HPLC t_{R} 5.46 min.

### Example 10

### Synthesis of Compound 10 N-(4-(3,5-dichlorobenzylamino)-2-oxobutyl)-2-naphthamide

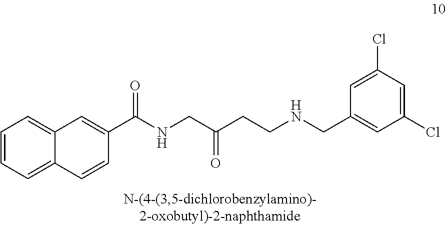 [SMILES: O=C(CCNCc1cc(Cl)cc(Cl)c1)CNC(=O)c1ccc2ccccc2c1]

To a solution of 3,5-dichlorobenzylamine (12 mg, 0.068 mmol) in DCM (0.2 mL) was added a solution of 9 (13 mg, 0.054 mmol) in DCM (0.5 mL) at room temperature. The resulting mixture was stirred until all of the 9 had been consumed (within one hr) and then was used straight in the next reaction. MS (ESI) 415 (M+1); HPLC t_{R} 6.00 min.

### Example 11

### Synthesis of Compound 11 (S)—N-(4-(5-(3-Pbf-guanidino)-2-(Fmoc-amino)-N-(3,5-dichlorobenzyl)pentanamido)-2-oxobutyl)-2-naphthamide

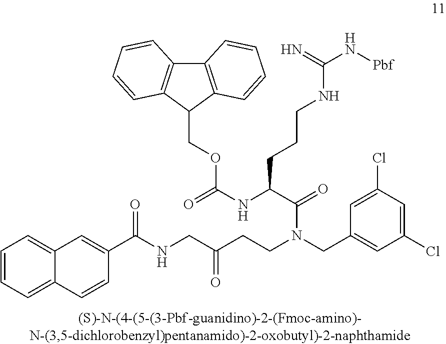

To a solution of freshly prepared aminoketone 10 in DCM (2 mL) was added Fmoc-L-Arg(Pbf)-OH (53 mg, 0.082 mmol) followed by DIC (12.5 μl, 0.082 mmol) at room temperature. The resulting mixture was stirred for 2 hrs then the solvent was removed under reduced pressure. The residue was filtered through a short plug of silica gel eluting with DCM followed by EtOAc to give the desired product 11 as a white solid. It was used in the next step without further purification. MS (ESI) 1045 (M+1); HPLC t_{R} 9.99 min.

### Example 12

### Synthesis of Compound 12 (S)—N-(4-(5-(3-Pbf-guanidino)-2-amino-N-(3,5-dichlorobenzyl)pentanamido)-2-oxobutyl)-2-naphthamide

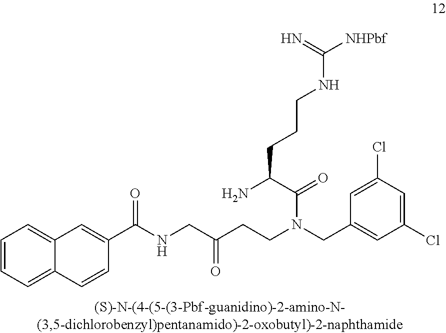

Diethylamine (0.5 mL) was added to Fmoc-protected 11 (56 mg, 0.054 mmol) at room temperature and the resulting mixture was stirred for 30 min. The excess amount of the diethylamine was removed under reduced pressure to give the desired free amine 12. It was used in the next step without further purification. MS (ESI) 823 (M+1); HPLC t_{R} 7.49 min.

### Example 13

### Synthesis of Compound 13 N-(((3S,5S)-3-(3-(3-Pbf-guanidino)propyl)-1-(3,5-dichlorobenzyl)-2-oxo-1,4-diazepan-5-yl)methyl)-2-naphthamide

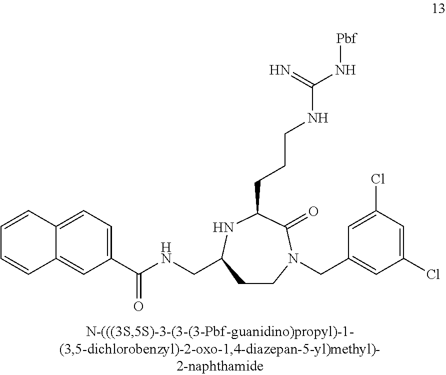

The amino ketone 12 (44 mg, 0.053 mmol) in DCM (2 mL) was cyclized by addition of NaBH(OAc)₃ (40 mg, 0.18 mmol) in one portion at room temperature. The resulting mixture was stirred for 3 hrs, followed by addition of saturated NaHCO₃ aqueous solution (3 mL). The aqueous layer was extracted with DCM (3×3 mL) and the combined organic layers were dried over MgSO₄, filtered and concentrated under reduced pressure. The residue was filtered through a short plug of silica gel eluting with DCM followed by EtOAc then EtOAc/IPA (9:1) to give the desired product 13 as a white solid. It was used in the next step without further purification. MS (ESI) 807 (M+1); HPLC t_{R} 7.75 min.

### Example 14

### Synthesis of Compound 100 N-(((3S,5S)-1-(3,5-dichlorobenzyl)-3-(3-guanidinopropyl)-2-oxo-1,4-diazepan-5-yl)methyl)-2-naphthamide

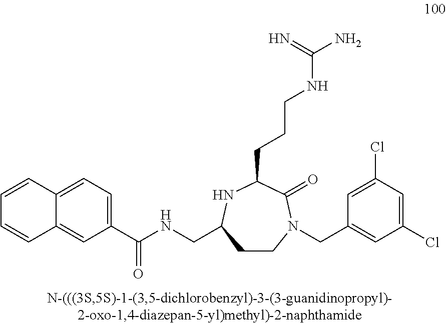 [SMILES: N=C(N)NCCC[C@@H]1N[C@H](CNC(=O)c2ccc3ccccc3c2)CCN(Cc2cc(Cl)cc(Cl)c2)C1=O]

A solution of TFA/DCM (2:1) (1 mL) with 5% H₂O was added to 13 at room temperature and the resulting mixture was stirred for 4 hrs. The solvents were removed under reduced pressure and the residue was purified by prep HPLC (100% H₂O to MeCN/H₂O 9:1, gradient) to give 100 (7.6 mg) as a white solid (TFA salt). The overall yield (from 9) was ca. 18%. MS (ESI) 556.2 (M+1); HPLC t_{R} 5.74 min.

### Example 15

### Synthesis of Compound 14 benzyl 2-(methoxy(methyl)amino)-2-oxoethylcarbamate

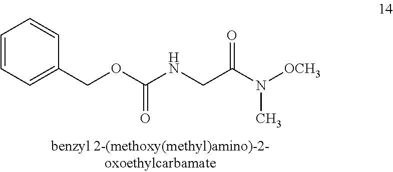 [SMILES: CON(C)C(=O)CNC(=O)OCc1ccccc1]

To Cbz-glycine (10 g, 47.8 mmol, Aldrich) in DCM (100 mL) was added BOP reagent (21.5 g, 48.6 mmol) and DIPEA (6.5 mL, 46.0 mmol). After stirring at room temperature for 10 mins, N,O-dimethylhydroxylamine hydrochloride (4.9 g, 50.2 mmol) and DIPEA (6.5 mL, 46.0 mmol) were added. The reaction was stirred at room temperature overnight. The DCM was removed by rotary evaporation and the residue taken up in EtOAc (100 mL). The organic phase was washed with H₂O (3×100 mL), saturated sodium bicarbonate solution (3×100 mL), H₂O (3×100 mL), 1M HCl (3×100 mL), brine (3×100 mL). The organic phase was dried (MgSO₄) and the EtOAc removed to give the Weinreb amide 14 as a white solid (7.78 g, 64%).

### Example 16

### Synthesis of Compound 15 benzyl 2-oxobut-3-enylcarbamate

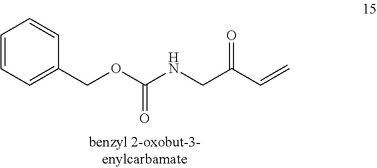 [SMILES: C=CC(=O)CNC(=O)OCc1ccccc1]

To the Weinreb amide 14 (3.89 g, 15.42 mmol) in DCM (10 mL) at 0° C. was added vinyl magnesium bromide (45 mmol) in THF (45 mL). The reaction was stirred for 2 hrs and monitored by HPLC. The reaction was added to a mixture of ice and 1M HCl (200 mL). The aqueous mixture was extracted with DCM (3×100 mL) and washed with 1M HCl (2×200 mL) and H₂O (3×100 mL). The organic phase was dried (MgSO₄) and the volume reduced to 100 mL by rotary evaporation. The α,β-unsaturated ketone 15 was stored and used in solution without purification.

### Example 17

### Synthesis of Compound 16 (S)-9-fluorenylmethyl 10-(2,2-diphenlethyl)-2,2-dimethyl-18-phenyl-4,9,13,16-tetraoxo-3,17-dioxa-5,10,15-triazaoctadecan-8-ylcarbamate

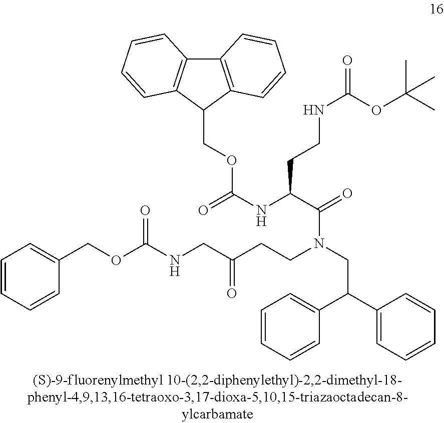 [SMILES: CC(C)(C)OC(=O)NCC[C@H](NC(=O)OCC1c2ccccc2-c2ccccc21)C(=O)N(CCC(=O)CNC(=O)OCc1ccccc1)CC(c1ccccc1)c1ccccc1]

To 2,2-diphenylethylamine (0.95 g, 7.4 mmol) in DCM (10 mL) was added the α,β-unsaturated ketone 15 (5.7 mmol) in DCM (75 mL). After stirring at room temperature for 15 mins, Fmoc-L-2,4-diaminobutyric acid(Boc)-OH (2.4 g, 8.55 mmol) and DIC (0.87 mL, 5.6 mmol) were added. The reaction was stirred at room temperature overnight. The DCM was removed by rotary evaporation and the residue was subjected to column chromatography on silica gel using petroleum spirit:EtOAc (1:1 to 0:1) to give 16 (1.5 g, 31%)

Alternatively, to 2,2-diphenylethylamine (0.97 g, 7.4 mmol) in DCM (20 mL) was added the α,β-unsaturated ketone 15 (5.95 mmol) in DCM (40 mL). After stirring at room temperature for 15 mins, Fmoc-L-2,4-diaminobutyric acid(Boc)-OH (2.4 g, 8.55 mmol), DIPEA (2.5 mL) and HATU (2.3 g, 6.0 mmol) were added. The reaction was stirred at room temperature overnight. The DCM was removed by rotary evaporation and the residue was taken up in EtOAc (100 mL). The organic layer was washed with saturated sodium bicarbonate solution (2×100 mL), saturated ammonium chloride solution (2×100 mL) and brine (2×100 mL). The organic phase was dried and the solvent removed under reduced pressure. The residue was subjected to column chromatography on silica gel using petroleum spirit:EtOAc (3:1 to 1:1 to 0:1) to give 16 (0.86 g, 17%).

### Example 18

### Synthesis of Compound 17 (3S,5S)-3-(2-tert-butoxycarbonylaminoethyl)-5-(benzyloxycarbonylaminomethyl)-1-(2,2-diphenylethyl)-1,4-diazepan-2-one

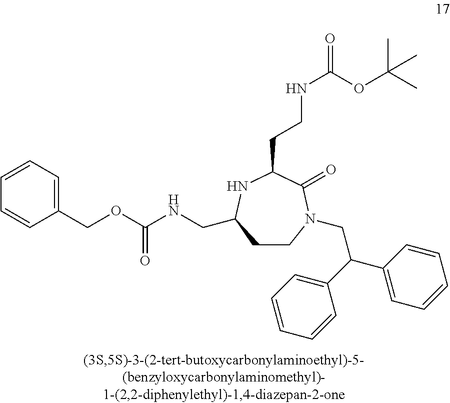 [SMILES: CC(C)(C)OC(=O)NCC[C@@H]1N[C@H](CNC(=O)OCc2ccccc2)CCN(CC(c2ccccc2)c2ccccc2)C1=O]

To Compound 16 (1.5 g, 1.8 mmol) in DCM (3 mL) was added diethylamine (1.5 mL, 14.5 mmol). The reaction was stirred at room temperature for 1 hr. The DCM and diethylamine was removed by rotary evaporation. DCM (5 mL), sodium triacetoxyborohydride (0.4 g, 1.9 mmol) was added, and the reaction was stirred overnight at room temperature. The organic phase was washed with saturated sodium bicarbonate solution (25 mL), dried (MgSO₄) and the DCM removed to give the cyclised product 17, which was used in the next step without purification.

### Example 19

### Synthesis of Compound 18 (3S,5S)-3-(2-tert-butoxycarbonylaminoethyl)-5-aminomethyl-1-(2,2-diphenylethyl)-1,4-diazepan-2-one

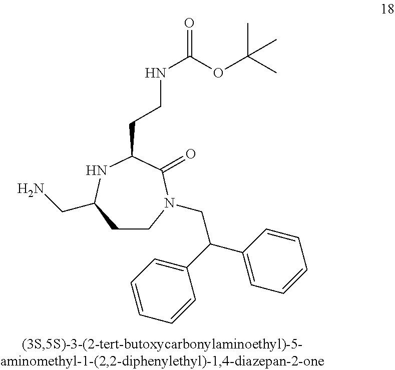 [SMILES: CC(C)(C)OC(=O)NCC[C@@H]1N[C@H](CN)CCN(CC(c2ccccc2)c2ccccc2)C1=O]

To the cyclised product 17 in methanol (5 mL) was added catalytic Pd/C. The reaction was stirred under a hydrogen atmosphere overnight. The reaction mixture was filtered through Celite and the methanol removed by rotary evaporation to give the amine 18 (0.7 g, 83% from 16).

### Example 20

### Synthesis of Compound 136 (E)-N-(((3S,5S)-3-(2-aminoethyl)-1-(2,2-diphenlethyl)-2-oxo-1,4-diazepan-5-yl)methyl)-3-(4-chlorophenyl)acrylamide

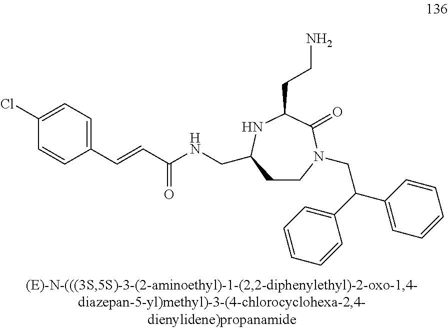 [SMILES: NCC[C@@H]1N[C@H](CNC(=O)/C=C/c2ccc(Cl)cc2)CCN(CC(c2ccccc2)c2ccccc2)C1=O]

To the amine 18 (0.06 g, 0.13 mmol) in DCM (5 mL) was added DIPEA (0.10 mL), BOP reagent (0.06 g, 0.13 mmol) and 4-chlorocinnamic acid (0.03 g, 0.16 mmol). The reaction was stirred at room temperature overnight. TFA (1 mL) was added and the reaction stirred at room temperature for 1 hrs. Rotary evaporation and preparative HPLC gave 136 (0.058 g, 84%). MS (ESI) 531.4 (M+1); HPLC t_{R} min 5.89

### Example 21

### Synthesis of Compound 101 (E)-3-(4-chlorophenyl)-N-(((3S,5S)-1-(2,2-diphenylethyl)-2-oxo-3-(2-(piperidin-1-yl)ethyl)-1,4-diazepan-5-yl)methyl)acrylamide

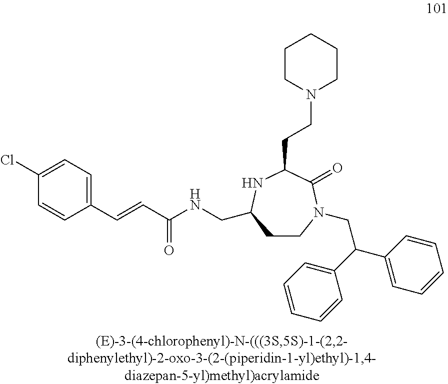 [SMILES: O=C(/C=C/c1ccc(Cl)cc1)NC[C@@H]1CCN(CC(c2ccccc2)c2ccccc2)C(=O)[C@H](CCN2CCCCC2)N1]

To the amine (E)-3-(4-chlorophenyl)-N-(((3S,5S)-1-(2,2-diphenylethyl)-2-oxo-3-(2-aminoethyl)-1,4-diazepan-5-yl)methyl)acrylamide (21 mg, 0.05 mmol) in DMF (0.25 mL) was added K₂CO₃ (5 mg) and 1,5-dibromopropane (0.066 mL, 0.5 mmol). The reaction mixture was left at room temperature for 4 hrs. The solvent was removed under high vacuum, and the residue purified by preparative HPLC to give 8 mg (˜30%) of 101 as the TFA salt. MS (ESI) 599.4 (M+1); HPLC t_{R} min 6.31

### Example 22

### Synthesis of Compound 19 (S)-9-fluorenylmethyl 10-(2-phenylbutyl)-2,2-dimethyl-18-phenyl-4,9,13,16-tetraoxo-3,17-dioxa-5,10,15-triazaoctadecan-8-ylcarbamate

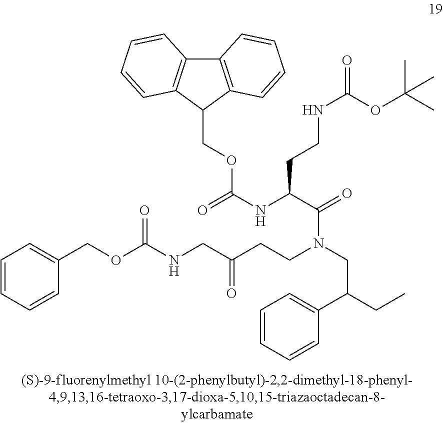 [SMILES: CCC(CN(CCC(=O)CNC(=O)OCc1ccccc1)C(=O)[C@H](CCNC(=O)OC(C)(C)C)NC(=O)OCC1c2ccccc2-c2ccccc21)c1ccccc1]

To 2-phenylbutylamine hydrochloride (0.26 g, 1.4 mmol) in DCM (10 mL) and DIPEA (0.25 mL, 1.8 mmol) was added the α,β-unsaturated ketone 15 (1.06 mmol) in DCM (20 mL). After stirring at room temperature for 15 mins, Fmoc-diaminobutyric acid(Boc)-OH (0.7 g, 1.56 mmol) and DIC (0.25 mL, 1.61 mmol) were added. The reaction was stirred at room temperature overnight. The DCM was removed by rotary evaporation and the residue was subjected to column chromatography on silica gel using petroleum spirit:EtOAc (1:1 to 0:1), providing Compound 19 as a mixture of diastereomers (0.17 g, 21%).

### Example 23

### Synthesis of Compound 20 (3S,5S)-3-(2-tert-butoxycarbonylaminoethyl)-5-(benzyloxycarbonylaminomethyl)-1-(2-phenylbutyl)-1,4-diazepan-2-one

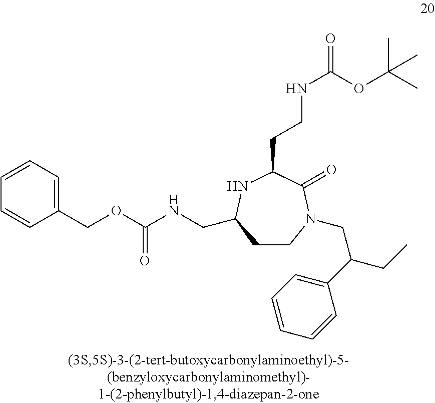 [SMILES: CCC(CN1CC[C@@H](CNC(=O)OCc2ccccc2)N[C@@H](CCNC(=O)OC(C)(C)C)C1=O)c1ccccc1]

To Compound 19 (0.17 g, 0.2 mmol) in DCM (3 mL) was added diethylamine (1.5 mL). The reaction was stirred at room temperature for 1 hr. The DCM and diethylamine was removed by rotary evaporation. DCM (5 mL) and sodium triacetoxyborohydride (0.1 g, 0.47 mmol) were added and the reaction was stirred overnight at room temperature. The organic phase was washed with saturated sodium bicarbonate solution (25 mL), dried (MgSO₄) and the DCM removed to give the cyclised product 20 as a mixture of diastereomers (0.11 g, 100%).

### Example 24

### Synthesis of Compound 21 (3S,5S)-3-(2-tert-butoxycarbonylaminoethyl)-5-(aminomethyl)-1-(2-phenylbutyl)-1,4-diazepan-2-one

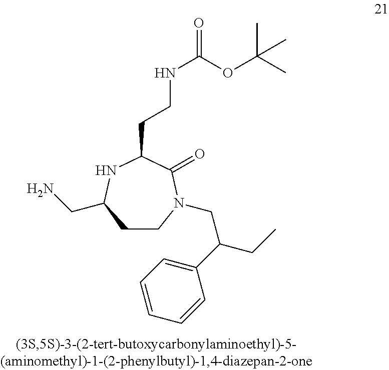 [SMILES: CCC(CN1CC[C@@H](CN)N[C@@H](CCNC(=O)OC(C)(C)C)C1=O)c1ccccc1]

To the cyclised product 20 (0.11 g) in methanol (5 mL) was added catalytic Pd/C. The reaction was stirred under a hydrogen atmosphere overnight. The reaction mixture was filtered through Celite and the methanol removed by rotary evaporation to give the amine 21 as a mixture of diastereomers (0.11 g, 100%).

### Example 25

### Synthesis of Compound 102 (3S,5S)-3-(2-aminoethyl)-5-(N-2-naphthamidomethyl)-1-(2-phenylbutyl)-1,4-diazepan-2-one

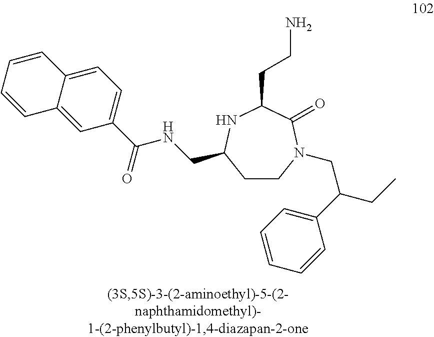 [SMILES: CCC(CN1CC[C@@H](CNC(=O)c2ccc3ccccc3c2)N[C@@H](CCN)C1=O)c1ccccc1]

To the amine 21 (0.02 mg, 0.05 mmol) in DCM (1 mL) was added DIPEA (0.1 mL, 0.7 mmol), BOP reagent (0.02 mg, 0.045 mmol) and 2-naphthoic acid (0.015 mg, 0.09 mmol). The reaction was stirred at room temperature for 2 hrs. TFA (1 mL) was added and the reaction stirred at room temperature for 2 hrs. Rotary evaporation and preparative HPLC gave 102 as a mixture of diastereomers (13.4 mg, 57%). MS (ESI) 473.4 (M+1); HPLC t_{R} 5.59 min

### Example 26

### Synthesis of Compound 22 allyl 2-(methoxy(methyl)amino)-2-oxoethylcarbamate

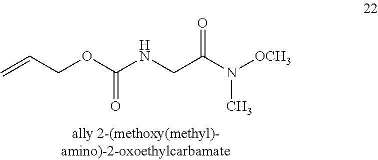

To Alloc-glycine (1.45 g, 9.1 mmol) in DCM (20 mL) was added BOP reagent (3.3 g, 7.46 mmol) and DIPEA (1.5 mL, 10.7 mmol). After stirring at room temperature for 10 mins, N,O-dimethylhydroxylamine hydrochloride (0.8 g, 8.2 mmol) and DIPEA (1.5 mL, 10.7 mmol) were added. The reaction was stirred at room temperature overnight. The DCM was removed by rotary evaporation and the residue taken up in EtOAc (100 mL). The organic phase was washed with H₂O (3×100 mL), saturated sodium bicarbonate solution (3×50 mL), H₂O (3×50 mL), 1M HCl (3×50 mL), brine (3×50 mL). The organic phase was dried (MgSO₄) and the EtOAc removed to give the Weinreb amide 22 as a white solid (0.43 g, 23%).

Alternatively, tert-butyl 2-(methoxy(methyl)amino)-2-oxoethylcarbamate 49 (Boc-Gly Weinreb amide, 1.4 g, 6.4 mmol) in DCM (5 mL) and TFA (3 mL) were stirred at room temperature 1 hr. The solvent was removed under reduced pressure, followed by addition of DCM (20 mL) and then DIPEA until basic. The solution of 2-amino-N-methoxy-N-methylacetamide (Gly Weinreb amide) was cooled to 0° C. and allyl chloroformate added (1.4 mL, 13.2 mmol). The reaction was stirred at room temperature overnight. The reaction mixture was neutralised with 1M HCl and extracted with EtOAc. The EtOAc was removed by rotary evaporation and the residue was subjected to column chromatography on silica gel using petroleum spirit:EtOAc (1:1 to 0:1), providing 22 (0.86 g, 66%).

### Example 27

### Synthesis of Compound 23 allyl 2-oxobut-3-enylcarbamate

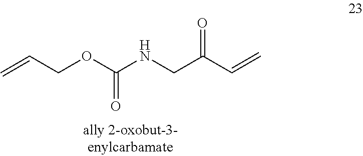

To the Weinreb amide 22 (0.43 g, 2.1 mmol) in DCM (5 mL) at 0° C. was added vinyl magnesium bromide (10 mmol) in THF (10 mL). The reaction was stirred for 2 hrs and monitored by HPLC. The reaction was added to a mixture of ice and 1M HCl (100 mL). The aqueous mixture was extracted with DCM (3×50 mL) and washed with 1M HCl (2×100 mL) and H₂O (3×50 mL). The organic phase was dried (MgSO₄) and the volume reduced to 50 mL by rotary evaporation. The α,β-unsaturated ketone 23 was stored and used in solution without further purification.

### Example 28

### Synthesis of Compound 24 (S)-9-fluorenylmethyl 10-(3,5-dichlorobenzyl)-2,2-dimethyl-4,9,13,16-tetraoxo-3,17-dioxa-5,10,15-triazaiscos-19-en-8-ylcarbamate

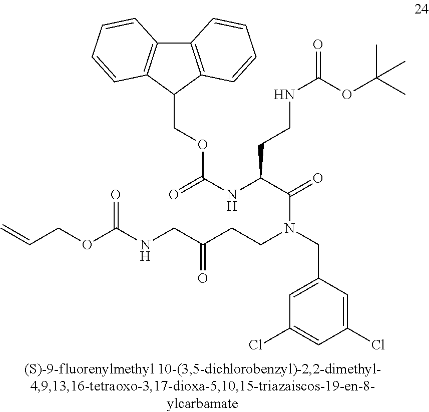 [SMILES: C=CCOC(=O)NCC(=O)CCN(Cc1cc(Cl)cc(Cl)c1)C(=O)[C@H](CCNC(=O)OC(C)(C)C)NC(=O)OCC1c2ccccc2-c2ccccc21]

To 3,5-dichlorobenzylamine (0.49 g, 2.8 mmol) in DCM (5 ml)-63 as added the α,β-unsaturated ketone 23 (2.12 mmol) in DCM (10 mL). After stirring at room temperature for 15 mins, Fmoc-diaminobutyric acid(Boc)-OH (1.35 g, 3.1 mmol) and DIC (0.5 mL, 3.2 mmol) was added. The reaction was stirred at room temperature overnight. The DCM was removed by rotary evaporation and the residue was subjected to column chromatography on silica gel using petroleum spirit:EtOAc (1:1 to 0:1), providing compound 24 (0.48 g, 22%).

### Example 29

### Synthesis of Compound 25 (3S,5S)-3-(2-tert-butoxycarbonylaminoethyl)-5-(allyloxycarbonylaminomethyl)-1-(3,5-dichlorobenzyll)-1,4-diazepan-2-one

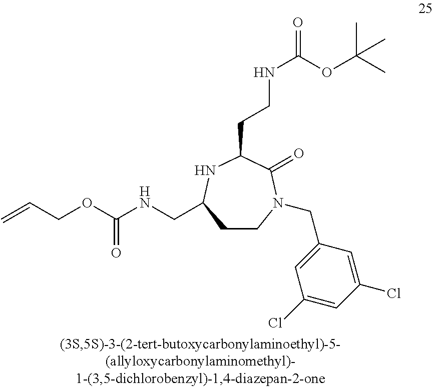 [SMILES: C=CCOC(=O)NC[C@@H]1CCN(Cc2cc(Cl)cc(Cl)c2)C(=O)[C@H](CCNC(=O)OC(C)(C)C)N1]

To Compound 24 (0.48 g, 0.63 mmol) in DCM (3 mL) was added diethylamine (1.5 mL). The reaction was stirred at room temperature for 1 hr. The DCM and diethylamine was removed by rotary evaporation. DCM (5 mL), sodium triacetoxyborohydride (0.2 g, 0.94 mmol) was added, and the reaction was stirred overnight at room temperature. The organic phase was washed with saturated sodium bicarbonate solution (25 mL), dried (MgSO₄) and the DCM removed to give the cyclised product 25 (0.24 g, 72%).

### Example 30

### Synthesis of Compound 26 (3S,5S)-3-(2-tert-butoxycarbonylaminoethyl)-5-aminomethyl-1-(3,5-dichlorobenzyl)-1,4-diazepan-2-one

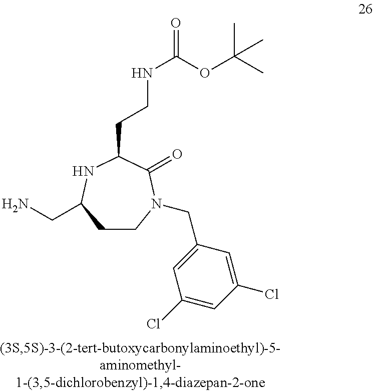 [SMILES: CC(C)(C)OC(=O)NCC[C@@H]1N[C@H](CN)CCN(Cc2cc(Cl)cc(Cl)c2)C1=O]

To the cyclised product 25 (0.24 g, 0.45 mmol) in DCM (3 mL) was added 1,3-dimethylbarbituric acid (13 mg, 0.08 mmol) and palladium tetrakis triphenylphosphine (5 mg). The reaction was evacuated and stirred and room temperature for 1 hr. The DCM was removed under reduced pressure to give the crude product 26 (0.15 g. 75%) which was used in the next reaction without purification.

### Example 31

### Synthesis of Compound 27 (3S,5S)-3-(2-aminoethyl)-5-(2-naphthoylaminomethyl)-1-(3,5-dichlorobenzyl)-1,4-diazepan-2-one

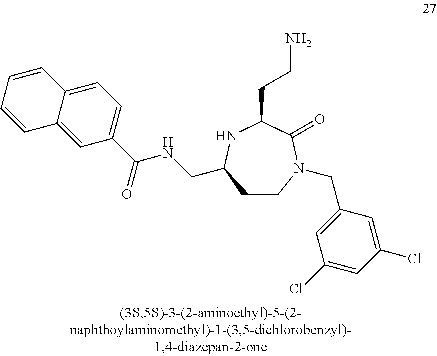 [SMILES: NCC[C@@H]1N[C@H](CNC(=O)c2ccc3ccccc3c2)CCN(Cc2cc(Cl)cc(Cl)c2)C1=O]

To the amine 26 (0.05 mg, 0.11 mmol) in DCM (1 mL) was added DIPEA (0.1 mL, 0.7 mmol), BOP reagent (0.05 mg, 0.11 mmol) and 2-naphthoic acid (0.04 mg, 0.23 mmol). The reaction was stirred at room temperature for 2 hrs. TFA (1 mL) was added and the reaction stirred at room temperature for 2 hrs. Rotary evaporation and preparative HPLC gave 27 (48 mg, 90%). MS (ESI) 499.3 (M+1); HPLC t_{R} 5.77 min

### Example 32

### Synthesis of Compound 103 N-(((3S,5S)-1-(2,2-diphenylethyl)-3-(3-guanidinopropyl)-2-oxo-1,4-diazepan-5-yl)methyl)-2-naphthalene-2-sulfonamide

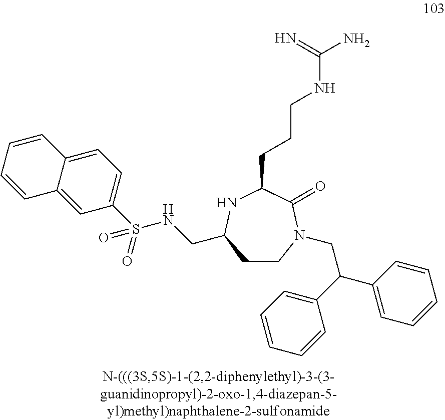 [SMILES: N=C(N)NCCC[C@@H]1N[C@H](CNS(=O)(=O)c2ccc3ccccc3c2)CCN(CC(c2ccccc2)c2ccccc2)C1=O]

Prepared from allyl 2-oxobut-3-enylcarbamate 23, Boc-L-Arg(Fmoc)₂—OH and 2,2-diphenylethylamine according the procedures of Examples 29-31, with the following modification: the Boc group was removed with TFA during the deprotection/cyclization procedure of Example 30, rather thane using diethylamine for Fmoc removal. Following Alloc deprotection by the procedure of Example 48, the free amine was dissolved in DCM to which was added naphthalene-2-sulfonyl chloride (10 mg) and DIPEA (20 μL) and the reaction stirred for 2 h at room temperature. Diethylamine (1 mL) was added and stirred overnight to remove the Fmoc protection, and the reaction evaporated to dryness. Preparative HPLC gave title compound 103 (13 mg). MS (ESI) 613.5 (M+1); HPLC t_{R} 5.89 min.

### Example 33

### Synthesis of Compound 28 (S)-9-fluorenylmethyl 10-(2-ethlbutyl)-2,2-dimethyl-18-phenyl-4,9,13,16-tetraoxo-3,17-dioxa-5,10,15-triazaoctadecan-8-ylcarbamate

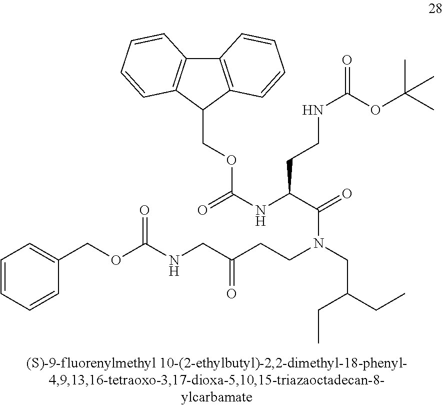 [SMILES: CCC(CC)CN(CCC(=O)CNC(=O)OCc1ccccc1)C(=O)[C@H](CCNC(=O)OC(C)(C)C)NC(=O)OCC1c2ccccc2-c2ccccc21]

To 2-ethylbutylamine (0.15 g, 1.48 mmol) in DCM (10 mL) was added the α,β-unsaturated ketone 15 (1.47 mmol) in DCM (30 mL). After stirring at room temperature for 15 mins, Fmoc-diaminobutyric acid(Boc)-OH (0.95 g, 2.16 mmol) and DIC (0.34 mL, 2.19 mmol) were added. The reaction was stirred at room temperature overnight. The DCM was removed by rotary evaporation and the residue was subjected to column chromatography on silica gel using petroleum spirit:EtOAc (1:1 to 0:1), providing Compound 28 (0.5 g, 46%).

### Example 34

### Synthesis of Compound 29 (3S,5S)-3-(2-tert-butoxycarbonylaminoethyl)-5-(benzyloxycarbonylaminomethyl)-1-(2-ethylbutyl)-1,4-diazepan-2-one

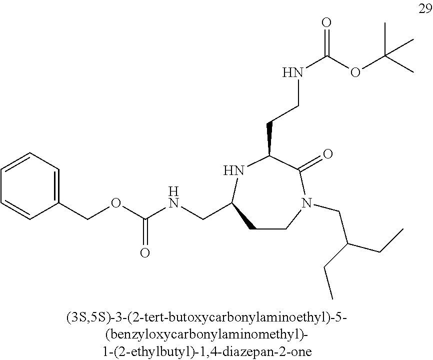 [SMILES: CCC(CC)CN1CC[C@@H](CNC(=O)OCc2ccccc2)N[C@@H](CCNC(=O)OC(C)(C)C)C1=O]

To Compound 28 (0.5 g, 0.67 mmol) in DCM (3 mL) was added diethylamine (1.5 mL). The reaction was stirred at room temperature for 1 hr. The DCM and diethylamine were removed by rotary evaporation. DCM (5 mL) and sodium triacetoxyborohydride (0.2 g, 0.94 mmol) were added and the reaction was stirred overnight at room temperature. The organic phase was washed with saturated sodium bicarbonate solution (25 mL), dried (MgSO₄) and the DCM removed to give the crude cyclised product 29 (0.4 g).

### Example 35

### Synthesis of Compound 30 (3S,5S)-3-(2-tert-butoxycarbonylaminoethyl)-5-(aminomethyl)-1-(2-ethylbutyl)-1,4-diazepan-2-one

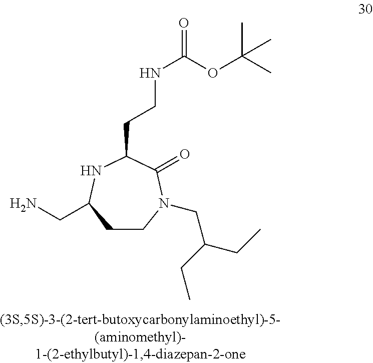 [SMILES: CCC(CC)CN1CC[C@@H](CN)N[C@@H](CCNC(=O)OC(C)(C)C)C1=O]

To the cyclised product 29 (0.4 g) in methanol (5 mL) was added catalytic Pd/C. The reaction was stirred under a hydrogen atmosphere overnight. The reaction mixture was filtered through Celite and the methanol removed by rotary evaporation to give the amine 30 (0.17 g, 68% from 28).

### Example 36

### Synthesis of Compound 146 (E)-N-(((3S,5S)-3-(2-aminoethyl)-1-(2-ethylbutyl)-2-oxo-1,4-diazepan-5-yl)methyl)-3-(4-chlorophenyl)acrylamide

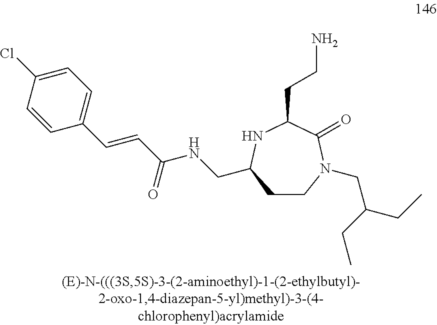 [SMILES: CCC(CC)CN1CC[C@@H](CNC(=O)/C=C/c2ccc(Cl)cc2)N[C@@H](CCN)C1=O]

To the amine 30 (0.020 g, 0.05 mmol) in DCM (3 mL) was added DIPEA (0.06 mL, 0.5 mmol), BOP reagent (0.02 g, 0.05 mmol) and 4-chlorocinnamic acid (0.015 g, 0.08 mmol). The reaction was stirred at room temperature for 2 hrs. TFA (0.5 mL) was added and the reaction stirred at room temperature for 1 hr. Rotary evaporation and preparative HPLC gave Compound 146 (23.5 mg, 95%). MS (ESI) 435.3 (M+1); HPLC t_{R} 5.46

### Example 37

### Synthesis of Compound 31 (3S,5S)-3-(2-tert-butoxycarbonylaminopropyl)-5-[benzyloxycarbonyl(methylamino)methyl]-1-(2,2 diphenylethyl)-1,4-diazepan-2-one

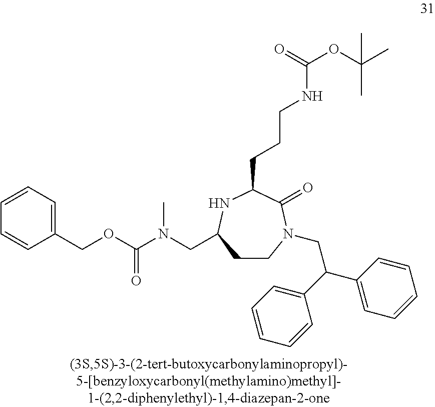 [SMILES: CN(C[C@@H]1CCN(CC(c2ccccc2)c2ccccc2)C(=O)[C@H](CCCNC(=O)OC(C)(C)C)N1)C(=O)OCc1ccccc1]

Prepared from Cbz-Sar, 2,2-diphenylethylamine and Fmoc-L-Orn(Boc) according to the procedures of Examples 16-19.

### Example 38

### Synthesis of Compound 32 (3S,5S)-3-(2-tert-butoxycarbonylaminopropyl)-5-(methylamino)methyl-1-(2,2-diphenylethyl)-1,4 diazepan-2-one

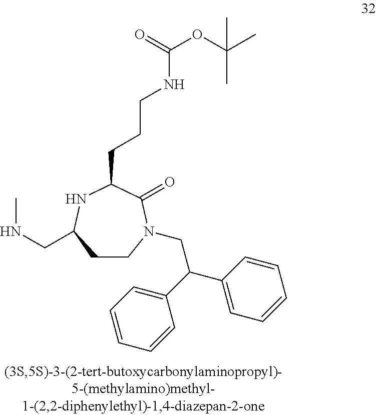 [SMILES: CNC[C@@H]1CCN(CC(c2ccccc2)c2ccccc2)C(=O)[C@H](CCCNC(=O)OC(C)(C)C)N1]

The cyclised product 31 (1.9 g) was dissolved in methanol (10 mL) with catalytic Pd/C and hydrogenated under a hydrogen atmosphere (40 psi) overnight. The reaction mixture was filtered through Celite and the methanol removed by rotary evaporation to give the amine 32 (1.86 g, 97%).

### Example 39

### Synthesis of Compound 104 N-(((3S,5S)-3-(3-aminopropyl)-1-(2,2-diphenylethyl)-2-oxo-1,4-diazepan-5-yl)methyl)-6-bromo-N-methyl-2-naphthamide

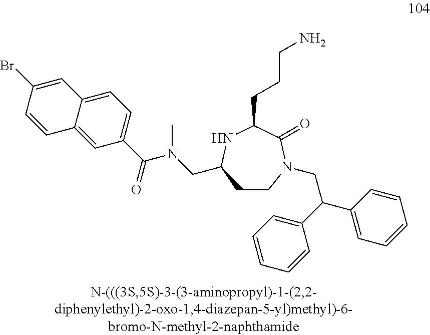 [SMILES: CN(C[C@@H]1CCN(CC(c2ccccc2)c2ccccc2)C(=O)[C@H](CCCN)N1)C(=O)c1ccc2cc(Br)ccc2c1]

The amine 32 was coupled with 6-bromo-2-naphthoic acid then deprotected with TFA according to Example 20. Rotary evaporation and preparative HPLC gave 104 (7.8 mg). MS (ESI) 629.4 (M+1); HPLC t_{R} 6.27 min.

### Example 40

### Synthesis of Compound 33 (3S,5S)-3-(tert-butoxycarbonylaminopropyl)-5-(6-bromo-2-naphthamidomethyl)-1-(2,2-diphenylethyl)-1,4-diazepan-2-one

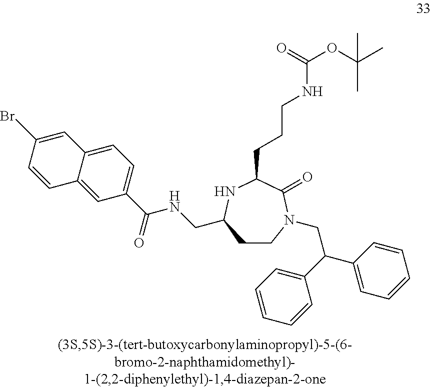 [SMILES: CC(C)(C)OC(=O)NCCC[C@@H]1N[C@H](CNC(=O)c2ccc3cc(Br)ccc3c2)CCN(CC(c2ccccc2)c2ccccc2)C1=O]

Prepared from 2,2-diphenylethylamine, Fmoc-L-Orn(Boc) and 6-bromo-2-naphthoic acid according to the procedures of Examples 17-20, without the TFA deprotection step of Example 20.

### Example 41

### Synthesis of Compound 105 N-(((3S,5S)-3-(3-aminopropyl)-1-(2,2-diphenlethyl)-4-methyl-2-oxo-1,4-diazepan-5-yl)methyl)-6-bromo-2-naphthamide

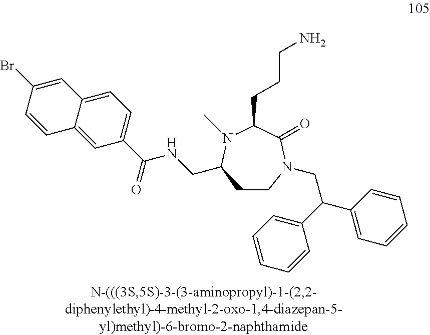 [SMILES: CN1[C@H](CNC(=O)c2ccc3cc(Br)ccc3c2)CCN(CC(c2ccccc2)c2ccccc2)C(=O)[C@@H]1CCCN]

Compound 33 (20.8 mg) was dissolved in DMF (1 mL) and treated with methyl iodide (6 μL) at room temperature for 1 week. Additional methyl iodide (0.5 mL) and K₂CO₃ were added and the reaction left at room temperature for an additional 2 days. TFA (2 mL) was added and the reaction stirred at room temperature for 2 h. Rotary evaporation followed by evaporation under high vacuum then preparative HPLC gave 105 (8.5 mg). MS (ESI) 629.3 (M+1); HPLC t_{R} 6.22 min.

### Example 42

### Synthesis of Compound 34 N-(((3S,5S)-3-(3-aminopropyl)-1-(2,2-diphenlethyl)-2-oxo-1,4-diazepan-5-yl)methyl)-2-naphthamide

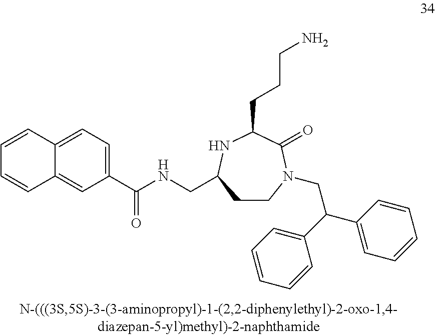 [SMILES: NCCC[C@@H]1N[C@H](CNC(=O)c2ccc3ccccc3c2)CCN(CC(c2ccccc2)c2ccccc2)C1=O]

Obtained from 9,2,2-diphenylethylamine and Fmoc-L-Orn(Boc) according to Examples 10-12. The Boc group was removed under standard conditions to give the free amine. MS (ESI) 535 (M+1); HPLC t_{R} 5.78 min

### Example 43

### Synthesis of Compound 106 N-(((3S,5S)-1-(2,2-diphenylethyl)-2-oxo-3-(3-(piperidin-1-yl)propyl)-1,4-diazepan-5-yl)methyl)-2-naphthamide

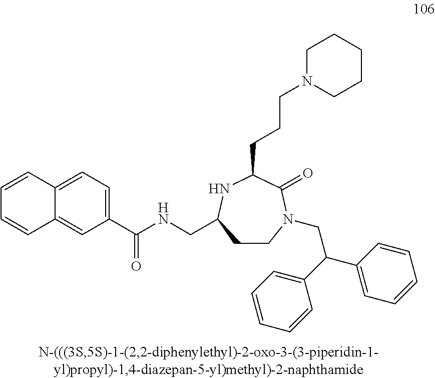 [SMILES: O=C(NC[C@@H]1CCN(CC(c2ccccc2)c2ccccc2)C(=O)[C@H](CCCN2CCCCC2)N1)c1ccc2ccccc2c1]

The amine 34 (0.79 g, 1.48 mmol), 1,5-dibromopentane (0.2 mL, 1.48 mmol) and K₂CO₃ (0.79 g) in DMF (11 mL) was stirred at room temperature for 4 h. The resulting mixture was diluted with ethylacetate (30 mL), washed with H₂O (5×30 mL), brine (10 mL) and dried over MgSO₄. Purification by preparative HPLC yielded 106 (0.23 g, 25%) MS (ESI) 603.3 (M+1); HPLC t_{R} 6.04 min

### Example 44

### Synthesis of Compound 35 (S)-2-(allyloxycarbonylamino)-3-(naphthalen-2-yl)propanoic acid

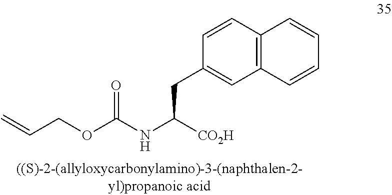 [SMILES: C=CCOC(=O)N[C@@H](Cc1ccc2ccccc2c1)C(=O)O]

To a stirred mixture of L-3-(2-naphthyl)alanine hydrochloride (5.0 g, 19.8 mmol), Na₂CO₃ (7.3 g, 69.3 mmol) and 1,4-dioxane (30 mL) in H₂O (50 mL) was added allylchloroformate (2.1 mL, 19.8 mmol) at 0° C. The resulting mixture was stirred for 16 h then concentrated under reduced pressure. The residue was diluted with ethylacetate (50 mL), and at 0° C. acidified to pH 2. The aqueous phase was extracted with ethylacetate (3×20 mL), the combined organic phase was washed with H₂O (50 mL) and brine (20 mL), dried over MgSO₄, filtered and concentrated under reduced pressure to give 35 as a colourless oil (5.8 g, 97%), which was used in the next step without further purification. HPLC t_{R} 6.60 min.

### Example 45

### Synthesis of Compound 36 (S)-allyl 1-(methoxy(methyl)amino)-3-(naphthalen-2-yl)-1-oxopropan-2-ylcarbamate

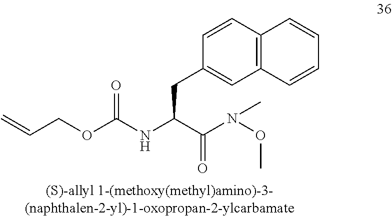 [SMILES: C=CCOC(=O)N[C@@H](Cc1ccc2ccccc2c1)C(=O)N(C)OC]

To a stirred mixture of the acid 35 (5.84 g, 19.5 mmol), DIPEA (3.7 mL, 2.09 mmol) and BOP (8.63 g, 19.5 mmol) in DCM (10 mL) was added a pre-mixed solution of N,O-dimethylhydroxylamine hydrochloride (1.9 g, 19.5 mmol) and DIPEA (7.3 mL, 41.6 mmol) in DCM (10 mL) at room temperature. Stirring continued for 16 h the reaction mixture was washed with 1N HCl (3×60 mL), H₂O (3×60 mL), saturated NaHCO₃ aqueous solution (3×60 mL) and brine (60 mL), dried over MgSO₄. Purification by silica gel chromatography using 20% ethylacetate in petroleum ether as eluent gave 36 (4.83 g, 71%) as a colourless oil. MS (ESI) 343 (M+1); HPLC t_{R} 7.07 min.

### Example 46

### Synthesis of Compound 37 (S)-allyl 1-(naphthalen-2-yl)-3-oxopent-4-en-2-ylcarbamate

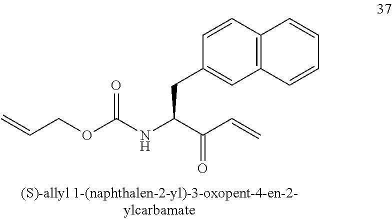 [SMILES: C=CCOC(=O)N[C@@H](Cc1ccc2ccccc2c1)C(=O)C=C]

At 0° C. a solution of vinylmagnesium bromide in THF (11.5 mL, 1 M) was added in one portion to Weinreb amide 36 (1.58 g, 4.62 mmol) under nitrogen with stirring. The resulting mixture was allowed to stir for 2 h, and poured into a 1N HCl/ice mixture (50 mL). The aqueous mixture was extracted with DCM (3×20 mL), the combined DCM extract was washed with 1N HCl (50 mL), saturated NaHCO₃ aqueous solution (50 mL) and brine (20 mL), dried over MgSO₄. Solvent was removed under reduced pressure producing the product 37 (1.14 g, 80%), which was used in the next step without further purification. MS (ESI) 310 (M+1); HPLC t_{R} 7.51 min.

### Example 47

### Synthesis of Compound 38 (S)-allyl 5-(2,2-diphenlethylamino)-1-(naphthalen-2-yl)-3-oxopentan-2-ylcarbamate

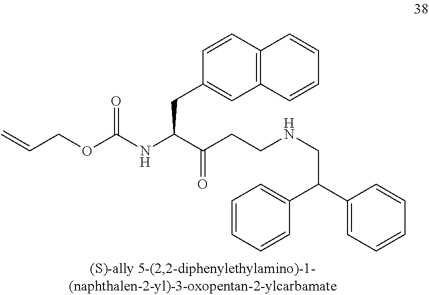 [SMILES: C=CCOC(=O)N[C@@H](Cc1ccc2ccccc2c1)C(=O)CCNCC(c1ccccc1)c1ccccc1]

To a stirred solution of 2,2-diphenylethylamine (0.45 g, 2.3 mmol) in DCM (55 mL) was added the vinyl ketone 37 (0.71 g, 2.3 mmol) in one portion. Stirring continued for 2 h, with the reaction mixture used in the next step without purification. MS (ESI) 507 (M+1); HPLC t_{R} 7.22 min.

### Example 48

### Synthesis of Compound 39 (S)-allyl 5-(N-(Boc-L-Arg(Cbz)₂) 2,2-diphenylethylamino)-1-(naphthalen-2-yl)-3-oxopentan-2-ylcarbamate

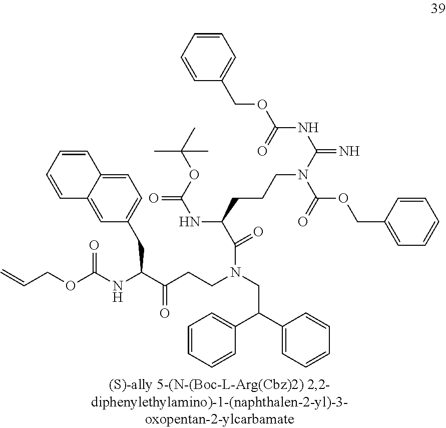 [SMILES: C=CCOC(=O)N[C@@H](Cc1ccc2ccccc2c1)C(=O)CCN(CC(c1ccccc1)c1ccccc1)C(=O)[C@H](CCCN(C(=N)NC(=O)OCc1ccccc1)C(=O)OCc1ccccc1)NC(=O)OC(C)(C)C]

To a stirred solution of the amine adduct 38 (2.3 mmol) was added a mixture of Boc-Arg(Cbz)₂—OH (1.25 g, 2.3 mmol), DIPEA (0.8 mL, 4.6 mmol) and HATU (0.87 g, 2.3 mmol) in DCM (15 mL) at room temperature. Stirring continued for 16 h, after which the reaction mixture was washed with saturated NaHCO₃ aqueous solution (3×20 mL) and brine (10 mL) then dried over MgSO₄. Purification by silica gel chromatography using 20% ethylacetate in petroleum ether as eluent gave 39 as a colourless oil (708 mg, 30% over 3 steps). MS (ESI) 1031 (M+1); HPLC t_{R} 10.80 min.

### Example 49

### Synthesis of Compound 40 alkyl (S)-1-((3S,5RS)-1-(2,2-diphenylethyl)-3-(bis Cbz 3-guanidinopropyl)-2-oxo-1,4-diazepan-5-yl)-2-(naphthalen-2-yl)ethylcarbamate

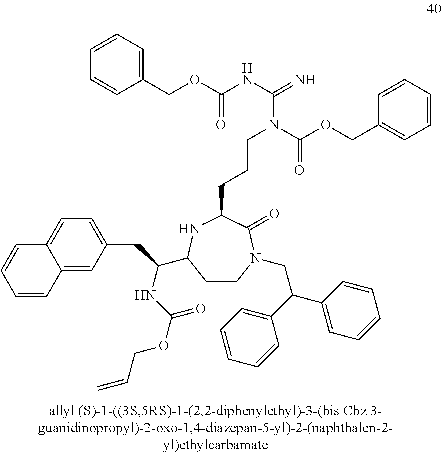 [SMILES: C=CCOC(=O)N[C@@H](Cc1ccc2ccccc2c1)C1CCN(CC(c2ccccc2)c2ccccc2)C(=O)[C@H](CCCN(C(=N)NC(=O)OCc2ccccc2)C(=O)OCc2ccccc2)N1]

To a stirred solution of acyclic intermediate 39 (0.48 g, 0.47 mmol) in DCM (5 mL) was added TFA (5 mL) at room temperature. Stirring continued for 30 min, after which the mixture was diluted with DCM (20 mL) then washed with saturated NaHCO₃ aqueous solution (3×20 mL) and brine (10 mL), and dried over MgSO₄. To the resulting solution was added sodium triacetoxyborohydride (0.2 g, 0.94 mmol) with stirring at room temperature, after 30 min the mixture was washed with saturated NaHCO₃ aqueous solution (3×20 mL) and brine (10 mL), then dried over MgSO₄. The crude 40, a mixture of diastereomers at the diazepan-2-one C5 position, was used in the next step without further purification. MS (ESI) 915 (M+1)

### Example 50

### Synthesis of Compound 41 bis(Cbz) 1-(3-((2S,7RS)-7-((S)-1-amino-2-(naphthalen-2-yl)ethyl)-4-(2,2-diphenlethyl)-3-oxo-1,4-diazepan-2-yl)propyl)guanidine

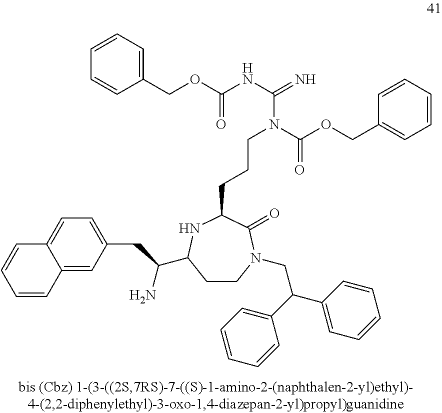 [SMILES: N=C(NC(=O)OCc1ccccc1)N(CCC[C@@H]1NC([C@@H](N)Cc2ccc3ccccc3c2)CCN(CC(c2ccccc2)c2ccccc2)C1=O)C(=O)OCc1ccccc1]

A mixture of compound 40 (36 mg, 0.039 mmol), 1,3-dimethylbarbituric acid (7.4 mg, 0.047 mmol) and Pd(PPh₃)₄ in DCM (5 mL) was stirred at room temperature under vacuum for 4 h. The resulting mixture was used in the next step without further purification. MS (ESI) 832 (M+1)

### Example 51

### Synthesis of Compounds 42 and 43 N—((S)-1-((3S,5S)-1-(2,2-diphenlethyl)-3-(3-guanidinopropyl)-2-oxo-1,4-diazepan-5-yl)-2-(naphthalen-2-yl)ethyl)acetamide and N—((S)-1-((3S,5R)-1-(2,2-diphenylethyl)-3-(3-guanidinopropyl)-2-oxo-1,4-diazepan-5-yl)-2-(naphthalen-2-yl)ethyl)acetamide

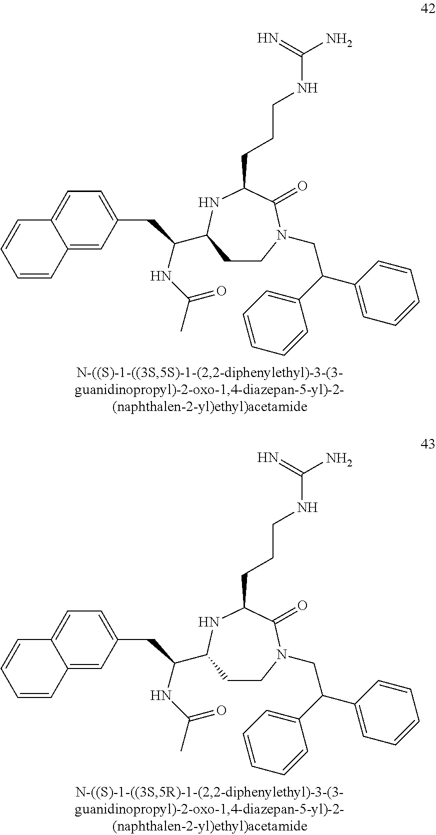 [SMILES: CC(=O)N[C@@H](Cc1ccc2ccccc2c1)[C@@H]1CCN(CC(c2ccccc2)c2ccccc2)C(=O)[C@H](CCCNC(=N)N)N1 ; CC(=O)N[C@@H](Cc1ccc2ccccc2c1)[C@H]1CCN(CC(c2ccccc2)c2ccccc2)C(=O)[C@H](CCCNC(=N)N)N1]

A solution of the amine 41 (0.09 mmol) in DCM (5 mL) was treated with acetic anhydride (8.6 μL, 0.09 mmol) with stirring at room temperature. After 3 h the mixture was concentrated, re-dissolved in EtOAc, washed with saturated NaHCO₃ aqueous solution (10 mL) and brine (10 mL), dried over MgSO₄, then concentrated under reduced pressure. The residue was dissolved in MeOH (10 mL), Pd/C (5 mg) was added, and the solution shaken under H₂ at 20 psi for 16 h. The reaction was filtered, concentrated and purified by preparative HPLC to give the minor diastereomer 42 (3 mg) and the major diastereomer 43 (6 mg) as white solids. 42: MS (ESI) 606.4 (M+1); HPLC t_{R} 6.033 min 43: MS (ESI) 606.3 (M+1); HPLC t_{R} 6.046 min

### Example 52

### Synthesis of Compounds 44 and 45 (S)-2-acetamido-N—((S)-1-((3S,5S)-1-(2,2-diphenylethyl)-3-(3-guanidinopropyl)-2-oxo-1,4-diazepan-5-yl)-2-(naphthalen-2-yl)ethyl)-3-(1H-imidazol-5-yl)propanamide and (S)-2-acetamido-N—((S)-1-((3S,5R)-1-(2,2-diphenylethyl)-3-(3-guanidinopropyl)-2-oxo-1,4-diazepan-5-yl)-2-(naphthalen-2-yl)ethyl)-3-(1H-imidazol-5-yl)propanamide

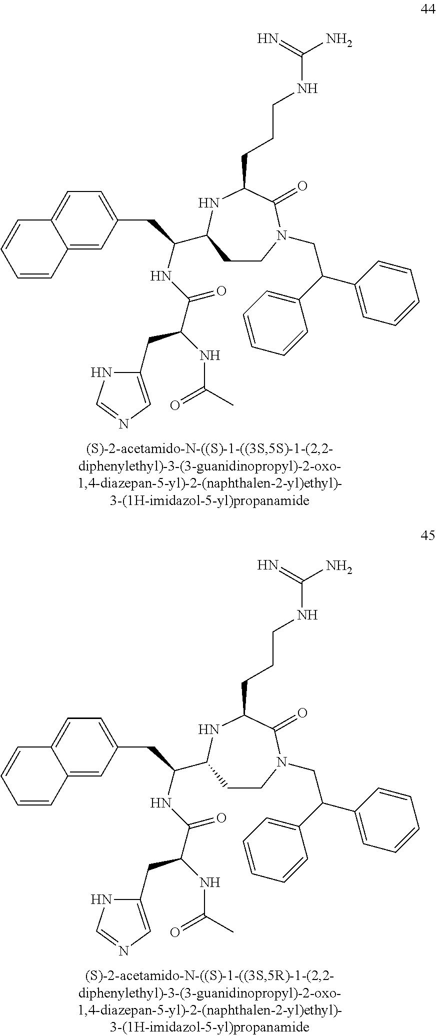 [SMILES: CC(=O)N[C@@H](Cc1cnc[nH]1)C(=O)N[C@@H](Cc1ccc2ccccc2c1)[C@@H]1CCN(CC(c2ccccc2)c2ccccc2)C(=O)[C@H](CCCNC(=N)N)N1 ; CC(=O)N[C@@H](Cc1cnc[nH]1)C(=O)N[C@@H](Cc1ccc2ccccc2c1)[C@H]1CCN(CC(c2ccccc2)c2ccccc2)C(=O)[C@H](CCCNC(=N)N)N1]

To a stirred mixture of Ac-L-His-OH (33.6 mg, 0.156 mmol), DIPEA (112.5 μL, 0.312 mmol) and BOP (68.8 mg, 0.156 mmol) in DMF (1 mL) was added the amine 41 (0.039 mmol) at room temperature. Stirring continued for 16 h, then the reaction mixture was diluted with DCM/H₂O mixture (10 mL, 1:1 v/v), and the aqueous phase was extracted with DCM (3×5 mL). The combined DCM extracts were washed with saturated NaHCO₃ aqueous solution (3×20 mL) and brine (10 mL), dried over MgSO₄, and concentrated under reduced pressure. The residue was re-dissolved in MeOH (5 mL), and Pd/C (20 mg) was added. The resulting mixture was shaken under H₂ at 30 psi for 16 h, then was filtered, concentrated and purified by preparative HPLC to give the minor diastereomer 44 (1.9 mg) and the major diastereomer 45 (0.9 mg) as white solids. 44: MS (ESI) 743.4 (M+1); HPLC t_{R} 5.489 min 45: MS (ESI) 743.4 (M+1); HPLC t_{R} 5.555 min

### Example 53

### Synthesis of Compounds 46 and 47 1-(3-((2S,7S)-7-(N—R1(R)-1-amino-2-(naphthalen-2-yl)ethyl)-4-(2,2-diphenlethyl)-3-oxo-1,4-diazepan-2-yl)propyl)guanidine

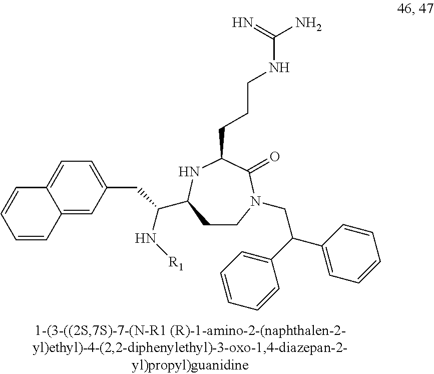

Compounds 46 and 47 were prepared in the same fashion as Compounds 42 and 44 using the procedures described in Examples 44-52, but with D-(2-naphthyl)alanine hydrochloride as the starting material.

| Compound | R₁ group | MS (M + 1) | t_{R} (min) |
|---|---|---|---|
| 46 | Ac | 606.2 | 6.01 |
| 47 | Ac-His | 743.5 | 5.41 |

### Examples 54-65

### Synthesis via Scheme 2: Preparation of all Four Diastereomers of N-((1-(2,2-diphenylethyl)-3-(3-guanidinopropyl)-2-oxo-1,4-diazepan-5-yl)methyl)-2-naphthamide

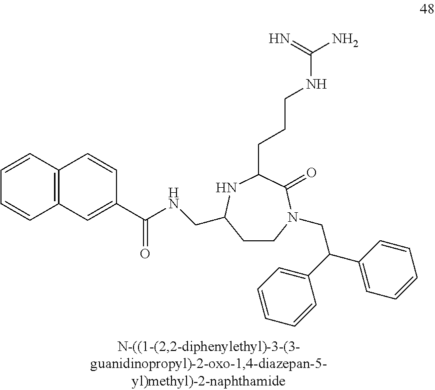 [SMILES: N=C(N)NCCCC1NC(CNC(=O)c2ccc3ccccc3c2)CCN(CC(c2ccccc2)c2ccccc2)C1=O]

### Example 54

### Synthesis of Compound 49 tert-butyl 2-(methoxy(methyl)amino)-2-oxoethylcarbamate (Boc-Gly Weinreb amide)

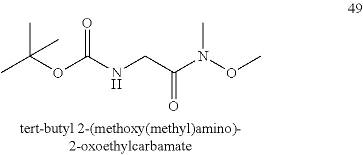

To a stirred mixture of Boc-Gly-OH (20 g, 114.1 mmol), DIPEA (19.8 mL, 114.1 mmol) and BOP (50.5 g, 114.1 mmol) in DCM (20 mL) was added a pre-mixed solution of N,O-dimethylhydroxylamine hydrochloride (11.2 g, 114.1 mmol) and DIPEA (19.8 mL, 114.1 mmol) in DCM (20 mL) at room temperature. The resulting mixture was stirred for 16 h then washed with 1N HCl (3×120 mL), H₂O (3×120 mL), saturated NaHCO₃ aqueous solution (3×120 mL) and brine (40 mL), dried over MgSO₄, filtered and concentrated under reduced pressure to give 49 as a white solid (20 g, 80%), which was used in the next step without further purification. MS (ESI) 219 (M+1); HPLC t_{R} 4.12 min.

### Example 55

### Synthesis of Compound 50 tert-butyl 2-oxobut-3-enylcarbamate

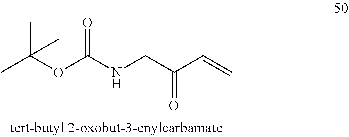

At 0° C. a solution of vinylmagnesium bromide in THF (184 mL, 1 M) was added in one portion to Weinreb amide 49 (20 g, 91.6 mmol) under nitrogen with stirring. The resulting mixture was allowed to stir for 2 h, and poured into a 1N HCl/ice mixture (400 mL). The aqueous mixture was extracted with DCM (5×100 mL), the combined DCM extract was washed with 1N HCl (2×100 mL), saturated NaHCO₃ aqueous solution (100 mL) and brine (100 mL), then dried over MgSO₄. Solvent was removed under reduced pressure gave the ketone 50 (12.9 g, 76%) as a pale yellow oil, which was used in the next step without further purification. MS (ESI) 186 (M+1); HPLC t_{R} 4.19 min.

### Example 56

### Synthesis of Compound 51 tert-butyl 4-(2,2-diphenylethylamino)-2-oxobutylcarbamate

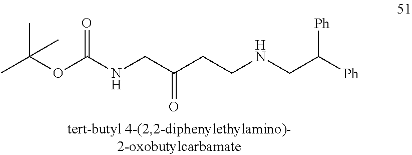 [SMILES: CC(C)(C)OC(=O)NCC(=O)CCNCC(c1ccccc1)c1ccccc1]

To a stirred solution of 2,2-diphenylethylamine (0.33 g, 1.66 mmol) in DCM (10 mL) was added α,β-unsaturated ketone 50 (0.31 g, 1.66 mmol) at room temperature. Stirring continued for 2 h; the crude reaction mixture of 51 was used in the next step without purification. MS (ESI) 383 (M+1); HPLC t_{R} 5.98 min

### Example 57

### Synthesis of Compound 52 2,2-dimethyl-10-(2,2-diphenlethyl)-4,7,11-trioxo-3,12-dioxa-5,10-diazapentadec-14-ene

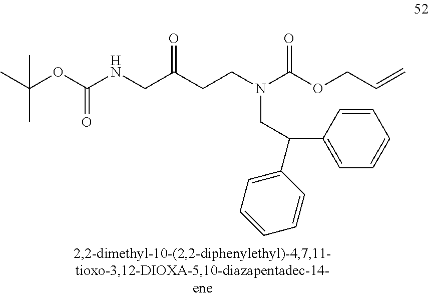 [SMILES: C=CCOC(=O)N(CCC(=O)CNC(=O)OC(C)(C)C)CC(c1ccccc1)c1ccccc1]

To the crude adduct 51 (prepared from 3 g 2,2-diphenylethylamine and 2.8 g Boc-vinylketone 50 as in Example 56) was added Alloc-C1 (1.6 mL) and the reaction stirred until TLC indicated consumption of the secondary amine. The solvent was evaporated and the residue purified by column chromatography (SiO₂ gel, pet. ether/EtOAc) to give 3.2 g (57%) of 52.

### Example 58

### Synthesis of Compound 53 (S)-allyl 2-amino-5-(benzyloxycarbonylamino)pentanoate L-H-Orn(Cbz)-Oallyl

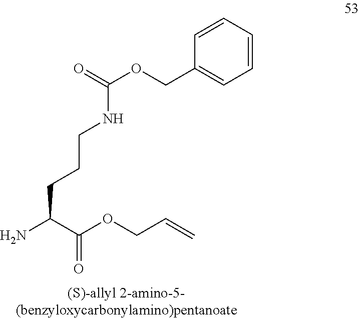 [SMILES: C=CCOC(=O)[C@@H](N)CCCNC(=O)OCc1ccccc1]

H-L-Orn(Cbz)-OH (6.66 g, 25 mmol), allyl alcohol (17.56 mL, 25 mmol) and p-TsOH (5.7 g, 30 mmol) were dissolved in benzene (200 mL) and refluxed under Dean-Stark conditions for 5 h. The majority of the solvent was then distilled off, with the remainder removed under vacuum. The resulting solid was recrystallized from DCM, filtered and dried to give 11.19 g (94%) of the tosylate salt. To obtain the free amine the solid was dissolved in DCM, washed with sat. NaHCO₃, the aqueous layer washed with DCM (3×), and the organic layers dried over MgSO₄ and evaporated to dryness.

### Example 59

### Synthesis of Compound 54 (R)-allyl 2-amino-5-(benzyloxycarbonylamino)pentanoate D-H-Orn(Cbz)-Oallyl

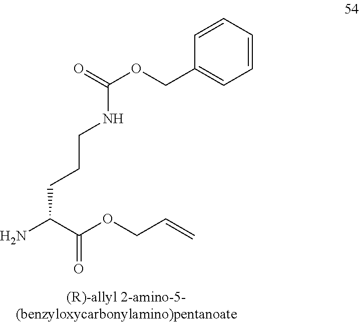 [SMILES: C=CCOC(=O)[C@H](N)CCCNC(=O)OCc1ccccc1]

H-D-Orn(Cbz)-OH (6.66 g, 25 mmol) was converted into 10.93 g (91%) of the tosylate salt of 54 as in Example 58, then converted into the free amine.

### Example 60

### Synthesis of Compound 55 (2R)-allyl 5-(benzyloxycarbonylamino)-2-(10-(2,2-diphenlethyl)-2,2-dimethyl-4,11-dioxo-3,12-dioxa-5,10-diazapentadec-14-en-7-ylamino)pentanoate

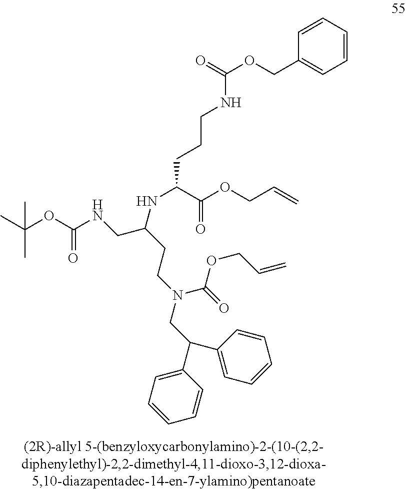

The protected aminoketone 52 (746 mg, 1.6 mmol), D-Om(Cbz)-Oallyl 54 (538 mg, 1.76 mmol) and NaBH(OAc)₃ (678 mg, 3.2 mmol) in a minimum volume of DCM were stirred for 24 h. A drop of AcOH was added just before workup, at which point saturated NaHCO₃ was added, extracted with DCM (3×), and the organic extracts combined and washed with saturated NaHCO₃ and H₂O, dried over MgSO₄, and evaporated to dryness. The product was purified by column chromatography (SiO₂ gel, pet. ether/EtOAc) to give 890 mg (74%) of 55 as a mixture of diastereoisomers.

### Example 61

### Synthesis of Compound 56 (2S)-allyl 5-(benzloxycarbonlamino)-2-(10-(2,2-diphenylethyl)-2,2-dimethyl-4,11-dioxo-3,12-dioxa-5,10-diazapentadec-14-en-7-ylamino)pentanoate

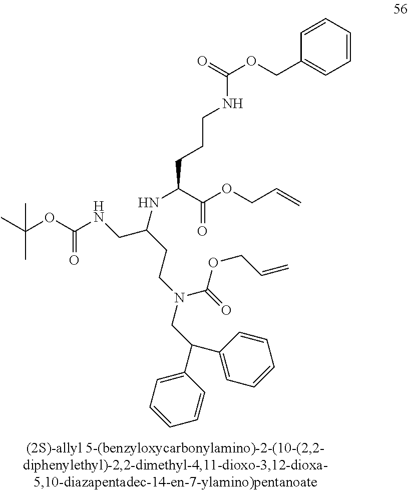 [SMILES: C=CCOC(=O)[C@H](CCCNC(=O)OCc1ccccc1)NC(CCN(CC(c1ccccc1)c1ccccc1)C(=O)OCC=C)CNC(=O)OC(C)(C)C]

Protected aminoketone 52 and L-Om(Cbz)-Oallyl 53 (592 mg, 1.93 mmol) were converted into a mixture of the set of diastereomers 56 (925 mg, 76%) following the procedures of Example 60.

### Example 62

### Synthesis of Compounds 57 and 58 (3R,5S)-5-(N-Boc aminomethyl)-3-(N-Cbz 3-aminopropyl)-1-(2,2-diphenylethyl)-1,4-diazepan-2-one and (3R,5R)-5-(N-Boc aminomethyl)-3-(N-Cbz 3-aminopropyl)-1-(2,2-diphenylethyl)-1,4-diazepan-2-one

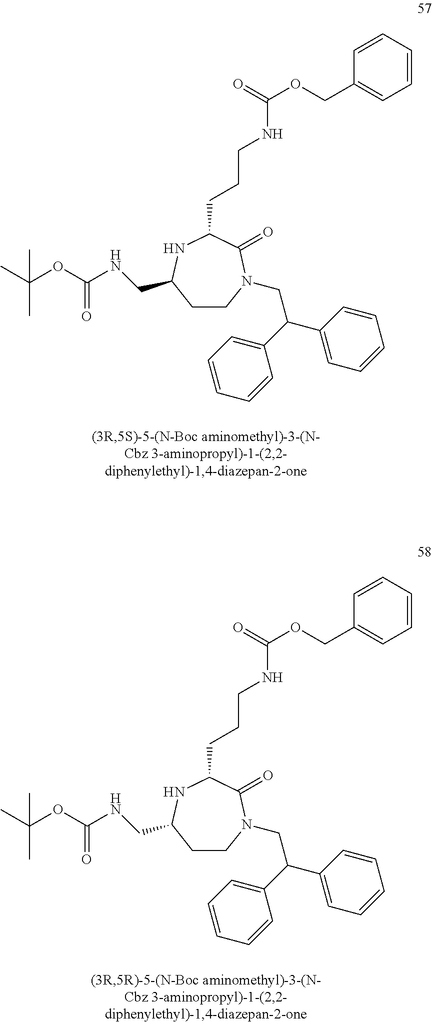 [SMILES: CC(C)(C)OC(=O)NC[C@@H]1CCN(CC(c2ccccc2)c2ccccc2)C(=O)[C@@H](CCCNC(=O)OCc2ccccc2)N1 ; CC(C)(C)OC(=O)NC[C@H]1CCN(CC(c2ccccc2)c2ccccc2)C(=O)[C@@H](CCCNC(=O)OCc2ccccc2)N1]

The Alloc/allyl protected derivative 55 (840 mg, 1.11 mmol) was dissolved in a minimum of DCM. 1,3-Dimethylbarbituric acid (346 mg, 2.22 mmol) and catalytic Pd(PPh₃)₄ were added, and the reaction degassed under vacuum, sealed and stirred overnight. The reaction was diluted to 50 mL with DCM, DIPEA (430 mg, 3.33 mmol) and BOP (540 mg, 1.22 mmol) were added, and the reaction stirred for 30 min. The DCM was removed under vacuum and the residue taken up in EtOAc, washed (saturated NaHCO₃, H₂O, saturated NaCl), dried (MgSO₄) and evaporated to dryness (TLC: EtOAc, 2 spots, R_{f} 0.33 and 0.57). The two diasteromeric products were separated by column chromatography (SiO₂ gel, pet. ether/EtOAc) to give 362 mg of the earlier eluting (3R,5S) isomer 57, and 342 mg of the later eluting (3R,5R) isomer 58.

### Example 63

### Synthesis of Compounds 59 and 60 (3S,5R)-5-(N-Boc aminomethyl)-3-(N-Cbz 3-aminopropyl)-1-(2,2-diphenylethyl)-1,4-diazepan-2-one and (3S,5S)-5-(N-Boc aminomethyl)-3-(N-Cbz 3-aminopropyl)-1-(2,2-diphenylethyl)-1,4-diazepan-2-one

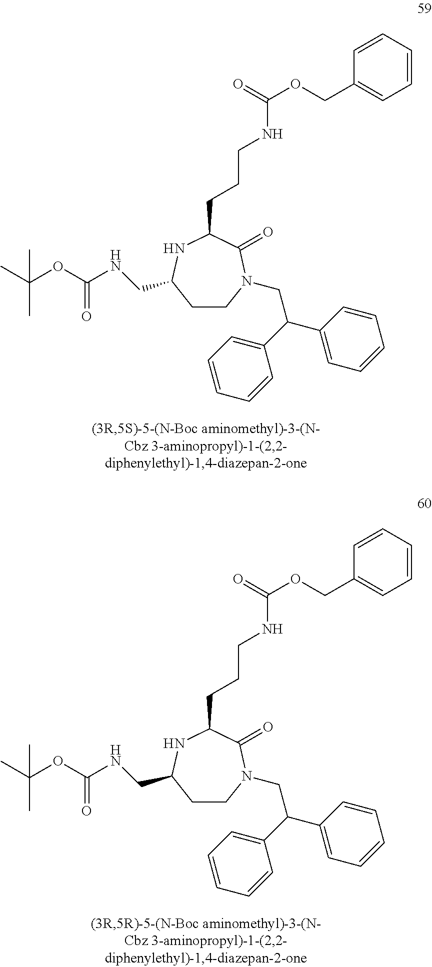 [SMILES: CC(C)(C)OC(=O)NC[C@H]1CCN(CC(c2ccccc2)c2ccccc2)C(=O)[C@H](CCCNC(=O)OCc2ccccc2)N1 ; CC(C)(C)OC(=O)NC[C@@H]1CCN(CC(c2ccccc2)c2ccccc2)C(=O)[C@H](CCCNC(=O)OCc2ccccc2)N1]

The (3S,5R) (312 mg) and (3S,5S) (331 mg) isomers were obtained from the L-Om-derived acyclic material 56 (870 mg) following the procedure of Example 62.

### Example 64

### Synthesis of Compounds 61-64 5-(N-Boc aminomethyl)-3-(N,N′-Cbz₂ 3-guanidinopropyl)-1-(2,2-diphenylethyl)-1,4-diazepan-2-one

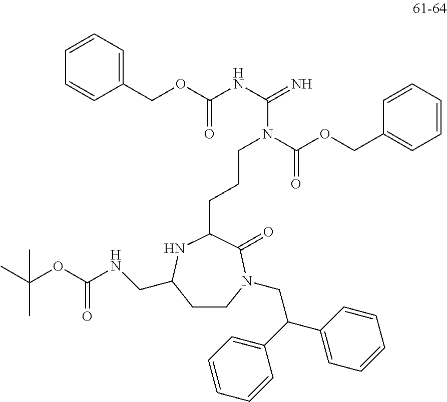 [SMILES: CC(C)(C)OC(=O)NCC1CCN(CC(c2ccccc2)c2ccccc2)C(=O)C(CCCN(C(=N)NC(=O)OCc2ccccc2)C(=O)OCc2ccccc2)N1]

The Orn Cbz group of 57 was removed by hydrogenation (H₂, 30 psi) over catalytic Pd/C in methanol overnight. The solution was filtered through Celite and evaporated to give a solid. A solution of the resulting amine (187 mg, 0.39 mmol) in DCM was mixed with a solution of the guanylating reagent CbzNHC(═NCbz)NHTf (196 mg, 0.43 mmol) in DCM. TEA (43 mg, 0.43 mmol) was added, and the reaction stirred overnight. The solution was diluted with DCM, washed (KHSO₄, sat. NaHCO₃, brine), dried (MgSO₄) and evaporated to dryness, then purified by flash chromatography over SiO₂ using hexanes/EtOAc as eluent, to give (3R,5S) 61 (182 mg, 59%). The other isomers 58-60 were converted in a similar manner to give:

62 (3R,5R): 171 mg (68%) from 148 mg of amine
63 (3S,5S): 80 mg (65%) from 72 mg of amine
64 (3S,5R): 142 mg (58%) from 144 mg of amine

### Example 65

### Synthesis of Compounds 65-67,107

65 N-(((3R,5S)-1-(2,2-diphenylethyl)-3-(3-guanidinopropyl)-2-oxo-1,4-diazepan-5-yl)methyl)-2-naphthamide
66 N-(((3R,5R)-1-(2,2-diphenylethyl)-3-(3-guanidinopropyl)-2-oxo-1,4-diazepan-5-yl)methyl)-2-naphthamide
67 N-(((3S,5R)-1-(2,2-diphenylethyl)-3-(3-guanidinopropyl)-2-oxo-1,4-diazepan-5-yl)methyl)-2-naphthamide
107 N-(((3S,5S)-1-(2,2-diphenylethyl)-3-(3-guanidinopropyl)-2-oxo-1,4-diazepan-5-yl)methyl)-2-naphthamide

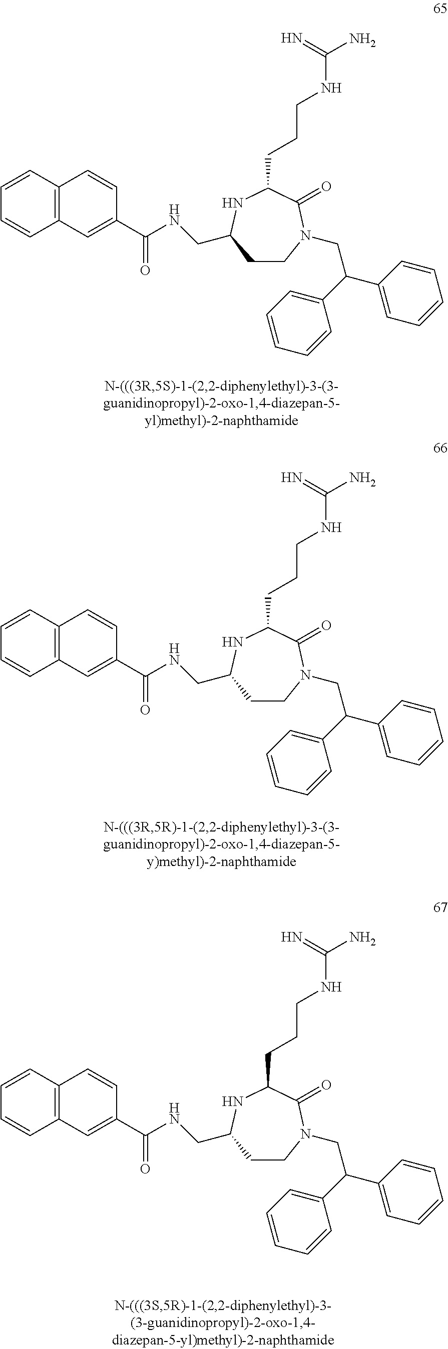 [SMILES: N=C(N)NCCC[C@H]1N[C@H](CNC(=O)c2ccc3ccccc3c2)CCN(CC(c2ccccc2)c2ccccc2)C1=O ; N=C(N)NCCC[C@H]1N[C@@H](CNC(=O)c2ccc3ccccc3c2)CCN(CC(c2ccccc2)c2ccccc2)C1=O ; N=C(N)NCCC[C@@H]1N[C@@H](CNC(=O)c2ccc3ccccc3c2)CCN(CC(c2ccccc2)c2ccccc2)C1=O]

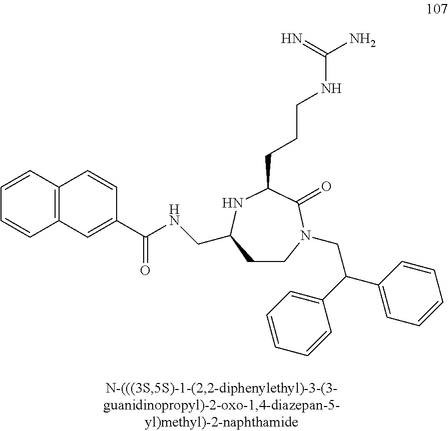 [SMILES: N=C(N)NCCC[C@@H]1N[C@H](CNC(=O)c2ccc3ccccc3c2)CCN(CC(c2ccccc2)c2ccccc2)C1=O]

The Boc derivative 62 (180 mg) in DCM (1 mL) was treated with TFA (1 mL) for 20 mL. The solvent was removed by evaporation, a solution of NaHCO₃ was added, and extracted 3× with DCM. The dichoromethane solution was dried over MgSO₄, filtered and evaporated to dryness. A portion (56 mg, 0.086 mmol) of the crude deprotected amine in DCM was stirred with 2-naphthoic acid (16 mg), DIPEA (60 uL) and BOP (42 mg) for 30 min. MeOH was added and the reaction stirred overnight. The reaction was filtered, then purified by flash chromatography over SiO₂ using petroleum ether/EtOAc as eluent, to give the Cbz-protected (3R,5R) isomer (43 mg, 94%). The other isomers were converted in a similar manner to give: (3R,5S): 41 mg (85%) from 60 mg 61, (3S,5R): 27 mg (70%) from 40 mg 64, and (3S,5S): 13 mg (74%) from 20 mg 63

Each compound was dissolved in dioxane:MeOH and hydrogenated over catalytic Pd/C under 30 psi H₂ overnight. The solution was filtered through Celite and evaporated to give a solid. 65 (3R,5S): 27 mg (96%) from 41 mg, 66 (3R,5R): 25 mg (85%) from 43 mg, 67 (3S,5R): 11 mg (quantitative) from 13 mg, and 107 (3S,5S): 3 mg (73%) from 6 mg.

| Compound | stereochemistry | MS (M + 1) | t_{R} (min) |
|---|---|---|---|
| 65 | (3R,5S) | 577.4 | 5.775 |
| 66 | (3R,5R) | 577.5 | 5.750 |
| 67 | (3S,5R) | 577.5 | 5.783 |
| 107 | (3S,5S) | 577.3 | 5.787 |

### Example 66

### Synthesis of Compound 686-chloro-2-naphthoic acid

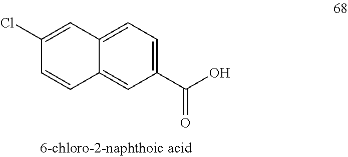 [SMILES: O=C(O)c1ccc2cc(Cl)ccc2c1]

A suspension of 6-bromo-2-naphthoic acid (3.0 g, 11.47 mmol), CuCl (11.7 g, 114.64 mmol) and CuI (2.19 g, 11.50 mmol) in degassed DMF (45 mL) was heated to reflux under argon in dark for 4 hrs. After cooling to room temperature, the solution was decanted into H₂O (200 mL) and the resulting mixture was extracted with EtOAc (2×500 mL). The combined organic layers were then washed with H₂O (4×500 mL) followed by brine (1×500 mL), dried over MgSO₄, filtered and concentrated under reduced pressure to dryness. The residue was trituated with CH₃CN and the solid obtained was then re-crystallized from EtOAc to give the pure product 68 (2.2 g, 93%) as a off-white solid. HPLC t_{R} 6.47 min.

### Example 67

### Synthesis of Compound 69 (S)-2-phenylbutanol

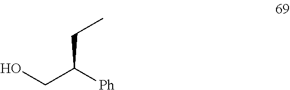

To a suspension of sodium borohydride (2.36 g, 62.4 mmol) in THF (50 mL) was added a solution of (S)-2-phenylbutyric acid (4.27 g, 26.0 mmol) in THF (40 mL) slowly at 0° C. The mixture was stirred until the evolution of gas ceased. A solution of iodine (6.60 g, 26.0 mmol) in THF (40 mL) was then added slowly at 0° C. After addition, the resulting mixture was allowed to warm to room temperature and stirred for 1 hr. The reaction solution was then slowly poured into a 1 N HCl solution (280 mL) and the resulting mixture was diluted with EtOAc (250 mL). The aqueous layer was extracted with EtOAc (150 mL×3) and the combined organic layers were then washed with saturated NaHCO₃ (aq), 0.5 M Na₂S₂O₃ (aq) and brine. This organic solution was dried over MgSO₄, filtered and concentrated under reduced pressure to give the crude product. Purification by flash chromatography on silica gel (Petroleum ether:EtOAc 4:1) gave the desired product 69 as a colorless oil in quantitative yield. HPLC t_{R} 5.24 min.

### Example 68

### Synthesis of Compound 70 (S)-1-mesyloxy-2-phenylbutane

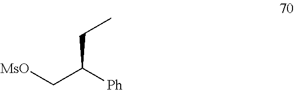

To a mixture of 69 (3.9 g, 26.0 mmol) and triethylamine (5.5 mL, 39.5 mmol) in DCM (90 mL) was added a solution of methanesulfonyl chloride (4.47 g, 39.0 mmol) in DCM (30 mL) slowly at 0° C. After addition, the resulting mixture was allowed to warm to room temperature and stirred for 2 hrs. 1 N HCl (70 mL) was then added to the above mixture and the aqueous layer was extracted with DCM (1×70 mL). The combined organic layers were washed with brine (150 mL), dried over MgSO₄, filtered and concentrated under reduced pressure to give the crude product 70 as a colorless oil. This crude product was used in the next step without further purification. HPLC t_{R} 6.48 min.

### Example 69

### Synthesis of Compound 71 (S)-1-azido-2-phenylbutane

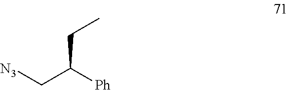

A suspension of 70 (5.93 g, 26.0 mmol) and sodium azide (5.7 g, 78.0 mmol) in DMF (60 mL) was heated at 85° C. for 3 hrs. After cooling to room temperature, the mixture was diluted with H₂O (200 mL) and extracted with EtOAc (250 mL). The organic layer was then washed with H₂O (4×150 mL) followed by brine (150 mL), dried over MgSO₄, filtered and concentrated under reduced pressure to give the crude product. Purification by flash chromatography on silica gel (100% petroleum ether as the eluent) gave the pure product 71 (4.03 g, 88%) as a colorless oil. HPLC t_{R} 7.67 min.

### Example 70

### Synthesis of Compound 72 (S)-2-phenylbutylamine

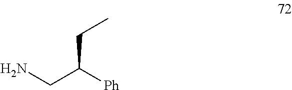

A mixture of 71 (4.0 g, 22.8 mmol) and Lindlar's catalyst (1.5 g) in EtOAc (50 mL) was shaken at room temperature under H₂ (40 psi) over-night. The mixture was then filtered through a pad of Celite and the filtrate was concentrated under reduced pressure to give the crude product 72 (3.4 g, 100%) as a light yellowish oil. This crude product was used for the conjugate addition reactions without further purification. MS (ESI) 150 (M+1); HPLC t_{R} 1.84 min.

### Example 71

### Synthesis of Compounds 73-78 182, 183

73 6-chloro-N-(((3S,5S)-2-oxo-1-((S)-2-phenylbutyl)-3-(2-(piperidin-1-yl)ethyl)-1,4-diazepan-5-yl)methyl)-2-naphthamide
74 6-chloro-N-(((3S,5S)-2-oxo-1-((R)-2-phenylbutyl)-3-(2-(piperidin-1-yl)ethyl)-1,4-diazepan-5-yl)methyl)-2-naphthamide
75 6-chloro-N-(((3S,5R)-2-oxo-1-((S)-2-phenylbutyl)-3-(2-(piperidin-1-yl)ethyl)-1,4-diazepan-5-yl)methyl)-2-naphthamide
76 6-chloro-N-(((3R,5S)-2-oxo-1-((S)-2-phenylbutyl)-3-(2-(piperidin-1-yl)ethyl)-1,4-diazepan-5-yl)methyl)-2-naphthamide
77 6-chloro-N-(((3S,5R)-2-oxo-1-((R)-2-phenylbutyl)-3-(2-(piperidin-1-yl)ethyl)-1,4-diazepan-5-yl)methyl)-2-naphthamide
78 6-chloro-N-(((3R,5S)-2-oxo-1-((R)-2-phenylbutyl)-3-(2-(piperidin-1-yl)ethyl)-1,4-diazepan-5-yl)methyl)-2-naphthamide
182 6-chloro-N-(((3R,5R)-2-oxo-1-((R)-2-phenylbutyl)-3-(2-(piperidin-1-yl)ethyl)-1,4-diazepan-5-yl)methyl)-2-naphthamide
183 6-chloro-N-(((3R,5R)-2-oxo-1-((S)-2-phenylbutyl)-3-(2-(piperidin-1-yl)ethyl)-1,4-diazepan-5-yl)methyl)-2-naphthamide

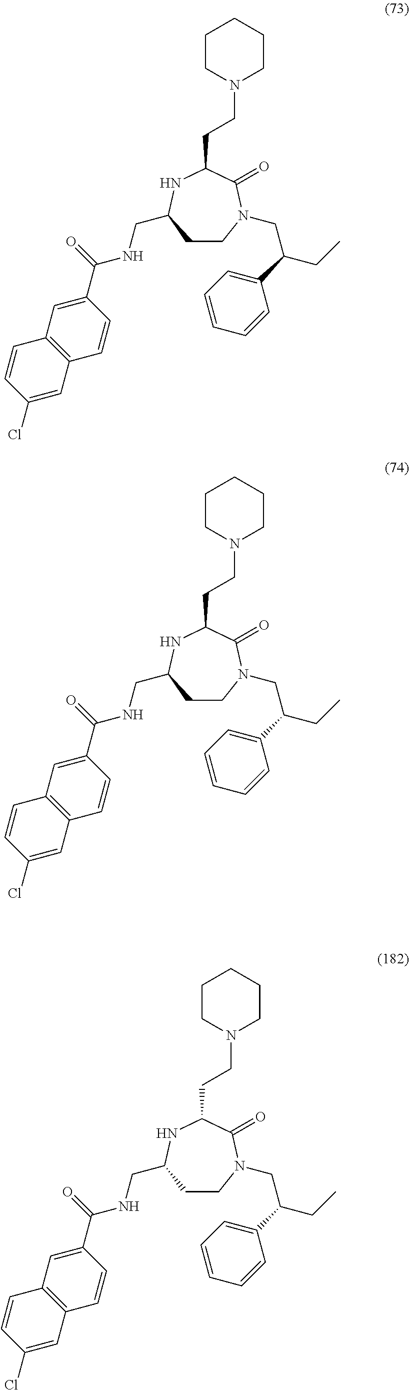 [SMILES: CC[C@H](CN1CC[C@@H](CNC(=O)c2ccc3cc(Cl)ccc3c2)N[C@@H](CCN2CCCCC2)C1=O)c1ccccc1 ; CC[C@@H](CN1CC[C@@H](CNC(=O)c2ccc3cc(Cl)ccc3c2)N[C@@H](CCN2CCCCC2)C1=O)c1ccccc1 ; CC[C@@H](CN1CC[C@H](CNC(=O)c2ccc3cc(Cl)ccc3c2)N[C@H](CCN2CCCCC2)C1=O)c1ccccc1]

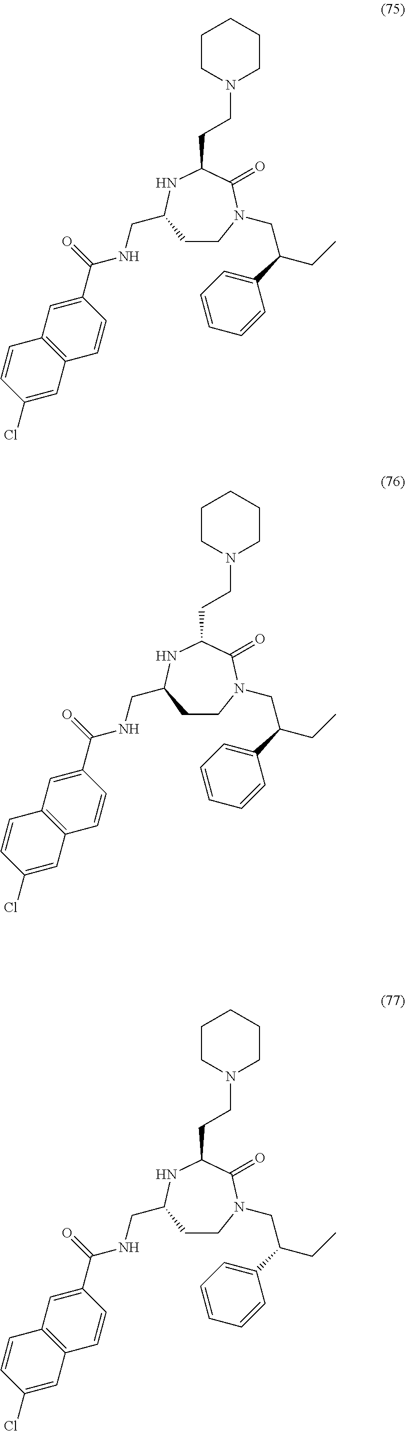 [SMILES: CC[C@H](CN1CC[C@H](CNC(=O)c2ccc3cc(Cl)ccc3c2)N[C@@H](CCN2CCCCC2)C1=O)c1ccccc1 ; CC[C@H](CN1CC[C@@H](CNC(=O)c2ccc3cc(Cl)ccc3c2)N[C@H](CCN2CCCCC2)C1=O)c1ccccc1 ; CC[C@@H](CN1CC[C@H](CNC(=O)c2ccc3cc(Cl)ccc3c2)N[C@@H](CCN2CCCCC2)C1=O)c1ccccc1]

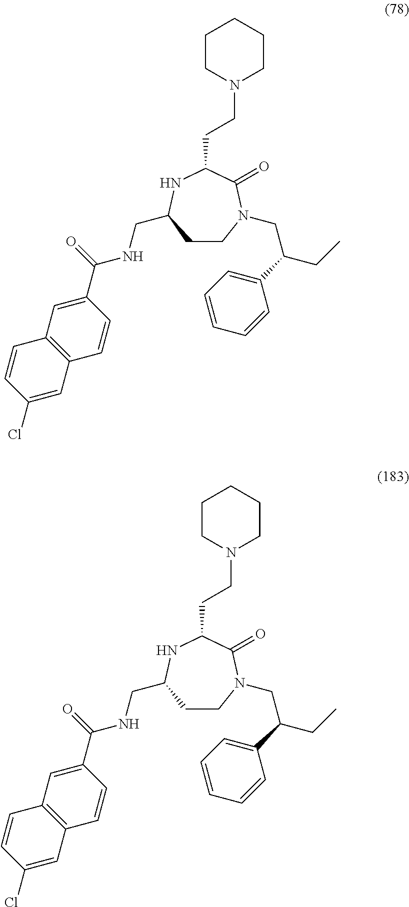 [SMILES: CC[C@@H](CN1CC[C@@H](CNC(=O)c2ccc3cc(Cl)ccc3c2)N[C@H](CCN2CCCCC2)C1=O)c1ccccc1 ; CC[C@H](CN1CC[C@H](CNC(=O)c2ccc3cc(Cl)ccc3c2)N[C@H](CCN2CCCCC2)C1=O)c1ccccc1]

Compounds 73-78, 182 and 183 were prepared following similar procedures as used to prepare Compounds 65-67 and 107 (Scheme 2 route). In addition, compounds 73, 74, 182 and 183 were also prepared according to the Scheme 1 route.

| Compound | stereochemistry | MS (M + 1) | t_{R} (min) |
|---|---|---|---|
| 73 | (3S,5S,2′S) | 575.3 | 6.269 |
| 74 | (3S,5S,2′R) | 574.8 | 6.265 |
| 182 | (3R,5R,2′R) | 575.4 | 6.404 |
| 75 | (3S,5R,2′S) | 575.2 | 6.262 |
| 76 | (3R,5S,2′S) | 575.2 | 6.110 |
| 77 | (3S,5R,2′R) | 575.1 | 6.211 |
| 78 | (3R,5S,2′R) | 575.2 | 6.253 |
| 183 | (3R,5R,2′S) | 575.4 | 6.274 |

### Example 72

### Syntheses of Compounds 100-186

Compounds 100-186, with substituents as identified in Table 2, were prepared as in the previous examples according to the routes identified in Schemes 1-5, as summarized in Table 3, with experimental properties summarized in Table 4.

TABLE 2
Identity of Compounds

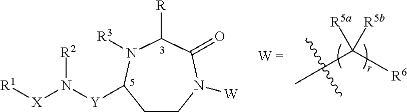

| | | | | | | | 3,5- |
|---|---|---|---|---|---|---|---|
| Cpd | R¹X | R² | R³ | Y | R | W | config |
| 100 | 2-naphthoyl | H | H | CH₂ | CH₂CH₂CH₂NHC(═NH)NH₂ | 3,5- | S,S |
| | | | | | | dichlorobenzyl | |
| 101 | 4- | H | H | CH₂ | CH₂CH₂(1-piperidinyl) | 2,2- | S,S |
| | chlorocinnamoyl | | | | | diphenylethyl | |
| 102 | 2-naphthoyl | H | H | CH₂ | CH₂CH₂NH₂ | 2-phenylbutyl | S,S |
| 103 | 2- | H | H | CH₂ | CH₂CH₂CH₂NHC(═NH)NH₂ | 2,2- | S,S |
| | naphthylsulfonyl | | | | | diphenylethyl | |
| 104 | 6-bromo-2- | Me | H | CH₂ | CH₂CH₂CH₂NH₂ | 2,2- | S,S |
| | naphthoyl | | | | | diphenylethyl | |
| 105 | 6-bromo-2- | H | Me | CH₂ | CH₂CH₂CH₂NH₂ | 2,2- | S,S |
| | naphthoyl | | | | | diphenylethyl | |
| 106 | 2-naphthoyl | H | H | CH₂ | CH₂CH₂CH₂(1-piperidinyl) | 2,2- | S,S |
| | | | | | | diphenylethyl | |
| 107 | 2-naphthoyl | H | H | CH₂ | CH₂CH₂CH₂NHC(═NH)NH₂ | 2,2- | S,S |
| | | | | | | diphenylethyl | |
| 108 | 4- | H | H | CH₂ | CH₂CH₂CH₂NHC(═NH)NH₂ | 2,2- | S,S |
| | biphenylcarbonyl | | | | | diphenylethyl | |
| 109 | quinoline-3- | H | H | CH₂ | CH₂CH₂CH₂NHC(═NH)NH₂ | 2,2- | S,S |
| | carbonyl | | | | | diphenylethyl | |
| 110 | 4- | R2 = R3 = | R2 = R3 = | CH₂ | CH₂CH₂CH₂NHC(═NH)NH₂ | diphenylmethyl | S,S |
| | biphenylmethyl | —CO— | —CO— | | | | |
| 111 | 3- | H | H | CH₂ | CH₂CH₂CH₂NHC(═NH)NH₂ | 2,2- | S,S |
| | phenoxybenzoyl | | | | | diphenylethyl | |
| 112 | 4- | H | H | CH₂ | CH₂CH₂CH₂NHC(═NH)NH₂ | 2,2- | S,S |
| | phenoxybenzoyl | | | | | diphenylethyl | |
| 113 | indole-2- | H | H | CH₂ | CH₂CH₂CH₂NHC(═NH)NH₂ | 2,2- | S,S |
| | carbonyl | | | | | diphenylethyl | |
| 114 | 4-tert- | H | H | CH₂ | CH₂CH₂CH₂NHC(═NH)NH₂ | 2,2- | S,S |
| | butylbenzoyl | | | | | diphenylethyl | |
| 115 | 1-methoxy-2- | H | H | CH₂ | CH₂CH₂CH₂NHC(═NH)NH₂ | 2,2- | S,S |
| | naphthoyl | | | | | diphenylethyl | |
| 116 | 2-naphthoyl | H | H | CH₂ | CH₂CH₂CH₂NHC(═NH)NH₂ | cyclohexane- | S,S |
| | | | | | | methyl | |
| 117 | 2-naphthoyl | H | H | CH₂ | CH₂CH₂CH₂NHC(═NH)N(Me)₂ | 2,2- | S,S |
| | | | | | | diphenylethyl | |
| 118 | N-acetyl (S)- | H | H | (S)- | CH₂CH₂CH₂NHC(═NH)NH₂ | (indol-3- | S,S |
| | histidine | | | CHBn | | yl)ethyl | |
| 119 | N-acetyl (S)- | H | H | (R)- | CH₂CH₂CH₂NHC(═NH)NH₂ | (indol-3- | S,S |
| | histidine | | | CHBn | | yl)ethyl | |
| 120 | 4-Me cinnamoyl | H | H | CH₂ | CH₂CH₂CH₂NH₂ | 2,2- | S,S |
| | | | | | | diphenylethyl | |
| 121 | 4- | H | H | CH₂ | CH₂CH₂CH₂NH₂ | 2,2- | S,S |
| | fluorocinnamoyl | | | | | diphenylethyl | |
| 122 | 6-fluoro-2- | H | H | CH₂ | CH₂CH₂CH₂NH₂ | 2,2- | S,S |
| | napthoyl | | | | | diphenylethyl | |
| 123 | 3,4- | H | H | CH₂ | CH₂CH₂CH₂NH₂ | 2,2- | S,S |
| | dichlorobenzoyl | | | | | diphenylethyl | |
| 124 | 2-naphthoyl | H | H | CH₂ | CH₂CH₂CH₂NHcHex | 2,2- | S,S |
| | | | | | | diphenylethyl | |
| 125 | 2-naphthoyl | H | H | CH₂ | CH₂CH₂CH₂NHC(═NH)NH₂ | 2-naphthyl | S,S |
| 126 | 2-naphthoyl | H | H | CH₂ | CH₂CH₂CH₂NHC(═NH)NH₂ | (9-fluorenyl)- | S,S |
| | | | | | | methyl | |
| 127 | 4- | H | H | CH₂ | CH₂CH₂CH₂NHcHex | 2,2- | S,S |
| | fluorocinnamoyl | | | | | diphenylethyl | |
| 128 | 5-(4- | H | H | CH₂ | CH₂CH₂CH₂NH₂ | 2,2- | S,S |
| | chlorophenyl)-2- | | | | | diphenylethyl | |
| | furoyl | | | | | | |
| 129 | 5-(4- | H | H | CH₂ | CH₂CH₂CH₂NH₂ | 2,2- | S,S |
| | chlorophenyl)- | | | | | diphenylethyl | |
| | isoxazole-3- | | | | | | |
| | carbonyl | | | | | | |
| 130 | 2-naphthoyl | H | H | CH₂ | CH₂CH₂CH₂NHC(═NH)NH₂ | cyclo- | S,S |
| | | | | | | hexaneethyl | |
| 131 | 2-naphthoyl | H | H | CH₂ | CH₂CH₂CH₂NHC(═NH)NH₂ | 2- | S,S |
| | | | | | | norbornaneethyl | |
| 132 | 4- | H | H | CH₂ | CH₂CH₂CH₂(1-piperidinyl) | 2,2- | S,S |
| | chlorocinnamoyl | | | | | diphenylethyl | |
| 133 | 4- | H | H | CH₂ | CH₂NH₂ | 2,2- | S,S |
| | chlorocinnamoyl | | | | | diphenylethyl | |
| 134 | 4- | H | H | CH₂ | CH₂(1-piperidinyl) | 2,2- | S,S |
| | chlorocinnamoyl | | | | | diphenylethyl | |
| 135 | 3,4- | H | H | CH₂ | CH₂CH₂NH₂ | 2,2- | S,S |
| | dichlorobenzoyl | | | | | diphenylethyl | |
| 136 | 4- | H | H | CH₂ | CH₂CH₂NH₂ | 2,2- | S,S |
| | chlorocinnamoyl | | | | | diphenylethyl | |
| 137 | 3,4- | H | H | CH₂ | CH₂CH₂(1-piperidinyl) | 2,2- | S,S |
| | dichlorobenzoyl | | | | | diphenylethyl | |
| 138 | 2-naphthoyl | H | H | CH₂ | CH₂CH₂CH₂NHC(═NH)NH₂ | CH₂CH(Ph)OPh | S,S |
| 139 | 2-naphthoyl | H | H | CH₂ | CH₂CH₂CH₂NHC(═NH)NH₂ | 3,5-dimethyl- | S,S |
| | | | | | | cyclohexylmethyl | |
| 140 | 3,4- | H | H | CH₂ | CH₂CH₂NH₂ | 3,5- | S,S |
| | dichlorobenzoyl | | | | | dichlorobenzyl | |
| 141 | 4- | H | H | CH₂ | CH₂CH₂NH₂ | 3,5- | S,S |
| | chlorocinnamoyl | | | | | dichlorobenzyl | |
| 142 | 4-fluorobenzyl | R2 = R3 = | R2 = R3 = | CH₂ | CH₂CH₂CH₂NHC(═NH)NH₂ | (S)- | S,S |
| | | —CO— | —CO— | | | CH(CONHMe)—CH₂(2- | |
| | | | | | | naphthyl | |
| 143 | 4-fluorobenzyl | H | H | CH₂ | CH₂CH₂CH₂NHC(═NH)NH₂ | (S)- | S,S |
| | | | | | | CH(CONHMe)—CH₂(2- | |
| | | | | | | naphthyl | |
| 144 | 2-naphthoyl | H | H | CH₂ | CH₂CH₂CH₂NH₂ | 1-(1-phenyl- | S,S |
| | | | | | | cyclohexyl)methyl | |
| 145 | 4- | H | H | CH₂ | CH₂CH₂(1-piperidinyl) | 3,5-dichlorobenzyl | S,S |
| | chlorocinnamoyl | | | | | | |
| 146 | 4- | H | H | CH₂ | CH₂CH₂NH₂ | 2-ethylbutyl | S,S |
| | chlorocinnamoyl | | | | | | |
| 147 | 2-naphthoyl | H | H | CH₂ | CH₂CH₂CH₂NHC(═NH)NH₂ | CH₂CH(Ph)CONHPh | S,S |
| 148 | 3,4- | H | H | CH₂ | CH₂NH₂ | 3,5- | S,S |
| | dichlorobenzoyl | | | | | dichlorobenzyl | |
| 149 | 2-naphthoyl | H | H | CH₂ | CH₂NH₂ | 3,5- | S,S |
| | | | | | | dichlorobenzyl | |
| 150 | 4- | H | H | CH₂ | CH₂NH₂ | 3,5- | S,S |
| | chlorocinnamoyl | | | | | dichlorobenzyl | |
| 151 | 3,4- | H | H | CH₂ | CH₂(1-piperidinyl) | 3,5- | S,S |
| | dichlorobenzoyl | | | | | dichlorobenzyl | |
| 152 | 4- | H | H | CH₂ | CH₂(1-piperidinyl) | 3,5- | S,S |
| | chlorocinnamoyl | | | | | dichlorobenzyl | |
| 153 | 4- | H | H | CH₂ | CH₂CH₂NH₂ | 2-phenylbutyl | S,S |
| | chlorocinnamoyl | | | | | | |
| 154 | 6-chloro-2- | H | H | CH₂ | CH₂CH₂(1-piperidinyl) | 2,2- | S,S |
| | naphthoyl | | | | | diphenylethyl | |
| 155 | 4-isopropyl- | H | H | CH₂ | CH₂CH₂(1-piperidinyl) | 2,2- | S,S |
| | cinnamoyl | | | | | diphenylethyl | |
| 156 | 4-isopropyl- | H | H | CH₂ | CH₂CH₂NH₂ | 2,2- | S,S |
| | cinnamoyl | | | | | diphenylethyl | |
| 157 | 2,4-dimethyl- | H | H | CH₂ | CH₂CH₂NH₂ | 2,2- | S,S |
| | cinnamoyl | | | | | diphenylethyl | |
| 158 | 2,4-difluoro- | H | H | CH₂ | CH₂CH₂(1-piperidinyl) | 2,2- | S,S |
| | cinnamoyl | | | | | diphenylethyl | |
| 159 | 4- | H | H | CH₂ | CH₂CH₂(4-morpholinyl) | 2,2- | S,S |
| | chlorocinnamoyl | | | | | diphenylethyl | |
| 160 | 4- | H | H | CH₂ | CH₂CH₂(2,5-Me₂- | 2,2- | S,S |
| | chlorocinnamoyl | | | | pyrrolidin-1-yl) | diphenylethyl | |
| 161 | 4- | H | H | CH₂ | CH₂CH₂(1-piperidinyl) | 2,2- | S,S |
| | bromocinnamoyl | | | | | diphenylethyl | |
| 162 | 5-(4-chlorophenyl)- | H | H | CH₂ | CH₂CH₂(1-piperidinyl) | 2,2- | S,S |
| | 3-isoxazole | | | | | diphenylethyl | |
| 163 | 6-chloro-2- | H | H | CH₂ | CH₂CH₂CH₂(1-piperidinyl) | (S)- | |
| | naphthoyl | | | | | phenylbutyl | S,S |
| 164 | 4- | H | H | C(Me)₂ | CH₂CH₂NH₂ | 2,2- | S,S |
| | chlorocinnamoyl | | | | | diphenylethyl | |
| 165 | 4- | H | H | C(Me)₂ | CH₂CH₂(1-piperidinyl) | 2,2- | S,S |
| | chlorocinnamoyl | | | | | diphenylethyl | |
| 166 | (S)-4-fluoro- | H | H | CH₂ | CH₂CH₂CH₂NHC(═NH)NH₂ | (S)- | S,R |
| | phenylalanine | | | | | CH(CONHMe)—CH₂(2- | |
| | | | | | | naphthyl) | |
| 167 | 4-chloro- | H | H | CH₂ | CH₂CH₂CH₂NHC(═NH)NH₂ | (S)- | S,R |
| | phenylacetic | | | | | CH(CONHMe)—CH₂(2- | |
| | | | | | | naphthyl) | |
| 168 | (R)-4-fluoro- | H | H | CH₂ | CH₂CH₂CH₂NHC(═NH)NH₂ | (S)- | S,R |
| | phenylalanine | | | | | CH(CONHMe)—CH₂(2- | |
| | | | | | | naphthyl) | |
| 169 | 6-chloro-2- | H | H | CH₂ | CH₂C(Me₂)NH₂ | (S)-2-phenylbutyl | R,R |
| | naphthoyl | | | | | | |
| 170 | 3,4- | H | H | CH₂ | CH₂CH₂NH₂ | (S)-2-phenylbutyl | S,S |
| | dichlorobenzoyl | | | | | | |
| 171 | 6-chloro-2- | H | H | CH₂ | CH₂C(Me₂)(1-piperidinyl) | (S)-2-phenylbutyl | S,S |
| | naphthoyl | | | | | | |
| 172 | 6-chloro-2- | H | H | CH₂ | CH₂C(Me₂)(1-piperidinyl) | (S)-2-phenylbutyl | R,R |
| | naphthoyl | | | | | | |
| 173 | (S)-4-chloro- | H | H | CH₂ | CH₂CH₂CH₂NHC(═NH)NH₂ | (S)- | S,R |
| | phenylalanine | | | | | CH(CONHMe)—CH₂(2- | |
| | | | | | | naphthyl) | |
| 174 | (R)-4-chloro- | H | H | CH₂ | CH₂CH₂CH₂NHC(═NH)NH₂ | (S)- | S,R |
| | phenylalanine | | | | | CH(CONHMe)—CH₂(2- | |
| | | | | | | naphthyl) | |
| 175 | 4- | H | H | CH₂ | CH₂CH₂CH₂NHC(═NH)NH₂ | (S)- | S,R |
| | chlorocinnamoyl | | | | | CH(CONHMe)—CH₂(2- | |
| | | | | | | naphthyl) | |
| 176 | 4- | H | H | CH₂ | CH₂CH₂CH₂NHC(═NH)NH₂ | CH₂cHex | R,R |
| | biphenylcarbonyl | | | | | | |
| 177 | 4- | H | H | CH₂ | CH₂CH₂(1-piperidinyl) | (S)-2- | S,S |
| | biphenylcarbonyl | | | | | phenylbutyl | |
| 178 | 2- | H | H | CH₂ | CH₂CH₂(1-piperidinyl) | (S)-2- | S,S |
| | phenylthiazole- | | | | | phenylbutyl | |
| | 4-carbonyl | | | | | | |
| 179 | 4-chloro- | H | H | CH₂ | CH₂CH₂(1-piperidinyl) | (S)-2- | S,S |
| | biphenyl-2- | | | | | phenylbutyl | |
| | carbonyl | | | | | | |
| 180 | 6-chloro-2- | H | H | CH₂ | CH₂CH₂N(Ac)iPr | (S)-2- | S,S |
| | naphthoyl | | | | | phenylbutyl | |
| 181 | 6-chloro-2- | H | H | CH₂ | CH₂CH₂(4-morpholinyl) | 2,2- | S,S |
| | naphthoyl | | | | | diphenylethyl | |
| 182 | 6-chloro-2- | H | H | CH₂ | CH₂CH₂(1-piperidinyl) | (R)-2- | S,S |
| | naphthoyl | | | | | phenylbutyl | |
| 183 | 6-chloro-2- | H | H | CH₂ | CH₂CH₂(1-piperidinyl) | (S)-2- | R,R |
| | naphthoyl | | | | | phenylbutyl | |
| 184 | 4- | H | H | CH₂ | CH₂CH₂CH₂CH₂NH₂ | 2-phenylethyl | R,S |
| | biphenylcarbonyl | | | | | | |
| 185 | 4- | H | H | CH₂ | CH₂CH₂CH₂NHC(═NH)NH₂ | diphenylmethyl | S,R |
| | phenylbenzoyl | | | | | | |
| 186 | 4- | H | H | CH₂ | CH₂CH₂CH₂NHC(═NH)NH₂ | diphenylmethyl | R,R |
| | phenylbenzoyl | | | | | | |

TABLE 3
Synthesis of Compounds

| | | | | Conversion | |
|---|---|---|---|---|---|
| | | Scheme 1: | | of A to | |
| Cpd. | Route to A | VN(R²)—Y—CO₂H | P²NH—CH(U)—CO₂H | Product | U modification |
| 100 | Scheme 1 | 2-naphthoic- | Fmoc-L-Arg(Pbf)-OH | Scheme 3 | P3 deprotection |
| | | Gly-OH | | | |
| 101 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 102 | Scheme 1 | Cbz-Gly-OH | Fmoc-L-Dab(Boc)—OH | Scheme 4 | P3 deprotection |
| 103 | Scheme 1 | Alloc-Gly-OH | Boc-L-Arg(Fmoc)₂—OH | Scheme 4 | P3 deprotection |
| 104 | Scheme 1 | Cbz-Sar | Fmoc-L-Orn(Boc)—OH | Scheme 4 | P3 deprotection |
| 105 | Scheme 1 | Cbz-Gly-OH | Fmoc-L-Orn(Boc)—OH | Scheme 3 | (ring methylate), |
| | | | | | P3 deprotect |
| 106 | Scheme 1 | 2-naphthoic- | Fmoc-L-Orn(Boc)—OH | Scheme 3 | P3 deprotection |
| | | Gly-OH | | | then dialkylation |
| | | | | | with alkyl |
| | | | | | dibromide |
| 107 | Scheme 2 | Boc-Gly-OH | H-L-Orn(Cbz)-Oallyl | Scheme 5 | P3 deprotection, |
| | | | | | guanidinylation, |
| | | | | | deprotection |
| 108 | Scheme 2 | Boc-Gly-OH | H-L-Orn(Cbz)-Oallyl | Scheme 5 | P3 deprotection, |
| | | | | | guanidinylation, |
| | | | | | deprotection |
| 109 | Scheme 2 | Boc-Gly-OH | H-L-Arg(Cbz)₂-Oallyl | Scheme 4 | P3 deprotection |
| 110 | Scheme 2 | Boc-Gly-OH | H-L-Arg(Cbz)₂-Oallyl | Scheme 4 | (R2 to R3 urea |
| | | | | | formation with |
| | | | | | phosgene) P3 |
| | | | | | deprotection |
| 111 | Scheme 2 | Boc-Gly-OH | H-L-Arg(Cbz)₂-Oallyl | Scheme 4 | P3 deprotection |
| 112 | Scheme 2 | Boc-Gly-OH | H-L-Arg(Cbz)₂-Oallyl | Scheme 4 | P3 deprotection |
| 113 | Scheme 2 | Boc-Gly-OH | H-L-Arg(Cbz)₂-Oallyl | Scheme 4 | P3 deprotection |
| 114 | Scheme 2 | Boc-Gly-OH | H-L-Arg(Cbz)₂-Oallyl | Scheme 4 | P3 deprotection |
| 115 | Scheme 1 | Alloc-Gly-OH | Boc-L-Arg(Fmoc)₂—OH | Scheme 4 | P3 deprotection |
| 116 | Scheme 1 | 2-naphthoic- | Fmoc-L-Arg(Pbf)-OH | Scheme 3 | P3 deprotection |
| | | Gly-OH | | | |
| 117 | Scheme 1 | 2-naphthoic- | Fmoc-L-Arg(NMe₂)Pbf-OH | Scheme 3 | P3 deprotection |
| | | Gly-OH | | | |
| 118 | Scheme 1 | Alloc-(S)-Phe | Boc-L-Arg(Cbz)₂—OH | Scheme 4 | P3 deprotection |
| 119 | Scheme 1 | Alloc-(R)-Phe | Boc-L-Arg(Cbz)₂—OH | Scheme 4 | P3 deprotection |
| 120 | Scheme 1 | Cbz-Gly-OH | Fmoc-L-Orn(Boc)—OH | Scheme 4 | P3 deprotection |
| 121 | Scheme 1 | Cbz-Gly-OH | Fmoc-L-Orn(Boc)—OH | Scheme 4 | P3 deprotection |
| 122 | Scheme 1 | Cbz-Gly-OH | Fmoc-L-Orn(Boc)—OH | Scheme 4 | P3 deprotection |
| 123 | Scheme 1 | Cbz-Gly-OH | Fmoc-L-Orn(Boc)—OH | Scheme 4 | P3 deprotection |
| 124 | Scheme 1 | 2-naphthoic- | Fmoc-L-Orn(Boc)—OH | Scheme 3 | P3 deprotection, |
| | | Gly-OH | | | reductive |
| | | | | | alkylation |
| 1257 | Scheme 1 | 2-naphthoic- | Fmoc-L-Arg(Pbf)-OH | Scheme 3 | P3 deprotection |
| | | Gly-OH | | | |
| 126 | Scheme 1 | 2-naphthoic- | Fmoc-L-Arg(Pbf)-OH | Scheme 3 | P3 deprotection |
| | | Gly-OH | | | |
| 127 | Scheme 1 | Cbz-Gly-OH | Fmoc-L-Orn(Boc)—OH | Scheme 4 | P3 deprotection, |
| | | | | | reductive |
| | | | | | alkylation |
| 128 | Scheme 1 | Cbz-Gly-OH | Fmoc-L-Orn(Boc)—OH | Scheme 4 | P3 deprotection |
| 129 | Scheme 1 | Cbz-Gly-OH | Fmoc-L-Orn(Boc)—OH | Scheme 4 | P3 deprotection |
| 130 | Scheme 1 | 2-naphthoic- | Fmoc-L-Arg(Pbf)-OH | Scheme 3 | P3 deprotection |
| | | Gly-OH | | | |
| 131 | Scheme 1 | 2-naphthoic- | Fmoc-L-Arg(Pbf)-OH | Scheme 3 | P3 deprotection |
| | | Gly-OH | | | |
| 132 | Scheme 1 | Cbz-Gly-OH | Fmoc-L-Orn(Boc)—OH | Scheme 4 | P3 deprotection |
| | | | | | then dialkylation |
| | | | | | with alkyl |
| | | | | | dibromide |
| 133 | Scheme 1 | Cbz-Gly-OH | Fmoc-L-Dap(Boc)—OH | Scheme 4 | P3 deprotection |
| 134 | Scheme 1 | Cbz-Gly-OH | Fmoc-L-Dap(Boc)—OH | Scheme 4 | P3 deprotection |
| | | | | | then dialkylation |
| | | | | | with alkyl |
| | | | | | dibromide |
| 135 | Scheme 1 | Cbz-Gly-OH | Fmoc-L-Dab(Boc)—OH | Scheme 4 | P3 deprotection |
| 136 | Scheme 1 | Cbz-Gly-OH | Fmoc-L-Dab(Boc)—OH | Scheme 4 | P3 deprotection |
| 137 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 138 | Scheme 1 | 2-naphthoic- | Fmoc-L-Arg(Pbf)-OH | Scheme 3 | P3 deprotection |
| | | Gly-OH | | | |
| 139 | Scheme 1 | 2-naphthoic- | Fmoc-L-Arg(Pbf)-OH | Scheme 3 | P3 deprotection |
| | | Gly-OH | | | |
| 140 | Scheme 1 | Aloc-Gly-OH | Fmoc-L-Dab(Boc)—OH | Scheme 4 | P3 deprotection |
| 141 | Scheme 1 | Alloc-Gly-OH | Fmoc-L-Dab(Boc)—OH | Scheme 4 | P3 deprotection |
| 142 | Scheme 2 | Cbz-Gly-OH | H-L-Arg(Pbf)-Oallyl | Scheme 4 | (R2 to R3 urea |
| | | | | | formation with |
| | | | | | phosgene) P3 |
| | | | | | deprotection |
| 143 | Scheme 2 | Cbz-Gly-OH | H-L-Arg(Pbf)-Oallyl | Scheme 4 | P3 deprotection |
| 144 | Scheme 1 | 2-naphthoic- | Fmoc-L-Orn(Boc)—OH | Scheme 3 | P3 deprotection |
| | | Gly-OH | | | |
| 145 | Scheme 1 | Alloc-Gly-OH | Fmoc-L-Dab(Boc)—OH | Scheme 4 | P3 deprotection |
| | | | | | then dialkylation |
| | | | | | with alkyl |
| | | | | | dibromide |
| 146 | Scheme 1 | Cbz-Gly-OH | Fmoc-L-Dab(Boc)—OH | Scheme 4 | P3 deprotection |
| 147 | Scheme 1 | 2-naphthoic- | Fmoc-L-Arg(Pbf)-OH | Scheme 3 | R5 deprotection |
| | | Gly-OH | | | and amidation then |
| | | | | | P3 deprotection |
| 148 | Scheme 1 | Alloc-Gly-OH | Fmoc-L-Dap(Boc)—OH | Scheme 4 | P3 deprotection |
| 149 | Scheme 1 | Alloc-Gly-OH | Fmoc-L-Dap(Boc)—OH | Scheme 4 | P3 deprotection |
| 150 | Scheme 1 | Cbz-Gly-OH | Fmoc-L-Dab(Boc)—OH | Scheme 4 | P3 deprotection |
| 151 | Scheme 1 | Cbz-Gly-OH | Fmoc-L-Dap(Boc)—OH | Scheme 5 | P3 deprotection |
| | | | | | then dialkylation |
| | | | | | with alkyl |
| | | | | | dibromide |
| 152 | Scheme 1 | Cbz-Gly-OH | Fmoc-L-Dap(Boc)—OH | Scheme 5 | P3 deprotection |
| | | | | | then dialkylation |
| | | | | | with alkyl |
| | | | | | dibromide |
| 153 | Scheme 1 | Cbz-Gly-OH | Fmoc-L-Dab(Boc)—OH | Scheme 4 | P3 deprotection |
| 154 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 155 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 156 | Scheme 1 | Cbz-Gly-OH | Fmoc-L-Dab(Boc)—OH | Scheme 4 | P3 deprotection |
| 157 | Scheme 1 | Cbz-Gly-OH | Fmoc-L-Dab(Boc)—OH | Scheme 4 | P3 deprotection |
| 158 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 159 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 160 | Scheme 1 | Cbz-Gly-OH | Fmoc-L-Dab(Boc)—OH | Scheme 4 | P3 deprotection, |
| | | | | | condensation, |
| | | | | | reduction |
| 161 | Scheme 1 | Cbz-Gly-OH | Fmoc-L-Dab(Boc)—OH | Scheme 5 | P3 deprotection |
| | | | | | then dialkylation |
| | | | | | with alkyl |
| | | | | | dibromide |
| 162 | Scheme 1 | Cbz-Gly-OH | Fmoc-L-Dab(Boc)—OH | Scheme 5 | P3 deprotection |
| | | | | | then dialkylation |
| | | | | | with alkyl |
| | | | | | dibromide |
| 163 | Scheme 1 | Cbz-Gly-OH | Fmoc-L-Orn(Boc)—OH | Scheme 5 | P3 deprotection |
| | | | | | then dialkylation |
| | | | | | with alkyl |
| | | | | | dibromide |
| 164 | Scheme 1 | Boc-Aib-OH | Fmoc-L-Dab(Boc)—OH | Scheme 4 | P3 deprotection |
| 165 | Scheme 1 | Boc-Aib-OH | Fmoc-L-Dab(Boc)—OH | Scheme 4 | P3 deprotection |
| | | | | | then dialkylation |
| | | | | | with alkyl |
| | | | | | dibromide |
| 166 | Scheme 2 | Cbz-Gly-OH | H-L-Arg(Pbf)-Oallyl | Scheme 4 | P3 deprotection |
| 167 | Scheme 2 | Cbz-Gly-OH | H-L-Arg(Pbf)-Oallyl | Scheme 4 | P3 deprotection |
| 168 | Scheme 2 | Cbz-Gly-OH | H-L-Arg(Pbf)-Oallyl | Scheme 4 | P3 deprotection |
| 169 | Scheme 1 | Cbz-Gly-OH | Fmoc-L-Dab(Boc) | Scheme 4 | P3 deprotection, |
| | | | | | dialkylation with |
| | | | | | alkyl dibromide |
| 170 | Scheme 1 | Cbz-Gly-OH | Fmoc-L-Dab(Boc) | Scheme 4 | P3 deprotection |
| 171 | Scheme 1 | Boc-Gly-OH | Cbz-DL-γ-nitro-Leu | Scheme 4 | P3 reduction to |
| | | | | | amine then |
| | | | | | dialkylation with |
| | | | | | alkyl dibromide |
| 172 | Scheme 1 | Boc-Gly-OH | Cbz-DL-γ-nitro-Leu | Scheme 4 | P3 reduction to |
| | | | | | amine then |
| | | | | | dialkylation with |
| | | | | | alkyl dibromide |
| 173 | Scheme 2 | Cbz-Gly-OH | H-L-Arg(Pbf)-Oallyl | Scheme 4 | P3 deprotection |
| 174 | Scheme 2 | Cbz-Gly-OH | H-L-Arg(Pbf)-Oallyl | Scheme 4 | P3 deprotection |
| 175 | Scheme 2 | Cbz-Gly-OH | H-L-Arg(Pbf)-Oallyl | Scheme 4 | P3 deprotection |
| 176 | Scheme 2 | Boc-Gly-OH | H-D-Arg(Cbz)2-Oallyl | Scheme 4 | P3 deprotection |
| 177 | Scheme 1 | Cbz-Gly-OH | Fmoc-L-Dab(Boc)—OH | Scheme 5 | P3 deprotection |
| | | | | | then dialkylation |
| | | | | | with alkyl |
| | | | | | dibromide |
| 178 | Scheme 1 | Cbz-Gly-OH | Fmoc-L-Dab(Boc)—OH | Scheme 5 | P3 deprotection |
| | | | | | then dialkylation |
| | | | | | with alkyl |
| | | | | | dibromide |
| 179 | Scheme 1 | Cbz-Gly-OH | Fmoc-L-Dab(Boc)—OH | Scheme 5 | P3 deprotection |
| | | | | | then dialkylation |
| | | | | | with alkyl |
| | | | | | dibromide |
| 180 | Scheme 1 | Cbz-Gly-OH | Fmoc-L-Dab(Boc)—OH | Scheme 4 | P3 deprotection |
| | | | | | then reductive |
| | | | | | alkylation then |
| | | | | | acetylation |
| 181 | Scheme 1 | Cbz-Gly-OH | Fmoc-L-Dab(Boc)—OH | Scheme 4 | P3 deprotection |
| | | | | | then dialkylation |
| | | | | | with alkyl |
| | | | | | dibromide |
| 182 | Scheme 1 | Cbz-Gly-OH | Fmoc-D-Dab(Boc)—OH | Scheme 4 | P3 deprotection |
| | | | | | then dialkylation |
| | | | | | with alkyl |
| | | | | | dibromide |
| 183 | Scheme 1 | Cbz-Gly-OH | Fmoc-D-Dab(Boc)—OH | Scheme 4 | P3 deprotection |
| | | | | | then dialkylation |
| | | | | | with alkyl |
| | | | | | dibromide |
| 184 | Scheme 2 | Boc-Gly-OH | H-D-Lys(Cbz)-Oallyl | Scheme 4 | P3 deprotection |
| 185 | Scheme 2 | Boc-Gly-OH | H-L-Arg(Cbz)₂-Oallyl | Scheme 4 | P3 deprotection |
| 186 | Scheme 2 | Boc-Gly-OH | H-D-Arg(Cbz)₂-Oallyl | Scheme 4 | P3 deprotection |

### Example 73

### Human MC1R Radioligand Binding Assay

Assessments of compound binding to human MC1R (hMC1R)) by displacement of an ¹²⁵I-labeled NDP-MSH receptor ligand peptide were performed essentially as described in the data sheets produced by Perkin Elmer to accompany their frozen hMC1R membranes (Perkin Elmer catalog number ES-195-M400UA).

[¹²⁵I] NDP-MSH: radiolabeled in house and purified by HPLC:

Na ¹²⁵I (0.5 mCi, 17.4 Ci/mg) was added to 50 μL sodium phosphate (50 mM, pH 7.4) in an eppendorf tube precoated with IODOGEN. After incubation for 10 mins the phosphate buffer containing the iodine was added to NDP-MSH (10 ul at 1 mg/mL) in a separate eppendorf tube. This was incubated for a further 10 mins. The iodinated NDP-MSH was purified by HPLC on a Zorbax SB 300 column using solvent A: 0.05% TFA and solvent B: 90% acetonitrile 0.045% TFA with a linear gradient, 0-67% B over 60 mins. The ¹²⁵I NDP-MSH eluted at 52 mins after the unlabeled starting material (48 min) and was counted and stored in the freezer. It was used within 48 hrs, as radioactive decay and ligand decomposition resulted in greatly reduced specific binding observed after 72 hrs.

### Reagents:

Incubation buffer: 25 mM HEPES-KOH (pH 7.0), 1.5 mM CaCl₂, 1 mM MgSO₄, 0.1 M NaCl, 1 mM 1,10-phenanthroline, and 1 Complete™ protease inhibitor tablet/100 mL (Roche, catalog number 1873580)

Perkin Elmer frozen hMC1 membranes: catalog number ES-195-M400UA, 0.4 mL/vial; 400 microassays/vial, 0.78 mg/mL protein concentration

Vials of frozen membranes were thawed rapidly immediately before use, diluted with binding buffer and vortexed. Resuspended membranes were kept on ice until they were added to the wells of the plate.

### Binding Protocol for 400 Microassays Per Vial:

Assays were performed in 96 well polypropylene plates. Membranes (0.78 μg 40 μL of a 1:40 dilution in incubation buffer) were added to [¹²⁵I]NDP-MSH (0.84 nM; 2200 Ci/mmol) and test compounds in a total volume of 140 μL. This was incubated for 1 hr at 37° C. Non-specific binding was determined with 3 mM NDP-MSH. Plates were filtered using a Tomtec cell harvester with GF/A filters (Wallac) (presoaked in 0.6% polyethylenimine) and washed three times with 1.0 mL ice-cold wash buffer (the above incubation buffer without 1,10-phenanthroline and Complete™ protease inhibitor tablet). The filters were dried in a 37° C. oven, placed in a sample bag and 5 mL Betaplatescint (Wallac) was added. Prepared filters were counted in cassettes in a Microbeta Trilux (Wallac) for 1 min. Non-specific binding just under 5%. Data analysis was performed using GraphPad Prism 4, employing competition binding with a single site model and a fixed Hill coefficient. The following equation was used: Y=Bottom+(Top-Bottom)/1/10̂(X−logEC₅₀), where X=log(concentration) and Y=binding to fit the data.

### Example 74

### Activity of Selected Compounds: hMC1R Binding

Representative compounds of the present invention were tested for binding in the hMC1R assay as in Example 73, as listed in Table 3. The compounds were tested as their trifluoroacetate or hydrochloride salts, or as their free base.

TABLE 4
Experimental Properties and MC1R
Radioligand Binding of Compounds

| Cpd. | MS (M + 1) | t_{R} (min) | MC1R radioligand IC₅₀ |
|---|---|---|---|
| 100 | 555.2 | 5.74 | x |
| 101 | 599.4 | 6.31 | x |
| 102 | 473.4 | 5.59 | x |
| 103 | 613.5 | 5.89 | x |
| 104 | 629.4 | 6.27 | x |
| 105 | 629.3 | 6.22 | x |
| 106 | 603.4 | 6.04 | x |
| 107 | 577.3 | 5.79 | x |
| 108 | 603.3 | 6.11 | x |
| 109 | 578.3 | 5.26 | x |
| 110 | 601.3 | 7.15 | x |
| 111 | 620.2 | 6.16 | x |
| 112 | 620.2 | 6.21 | x |
| 113 | 566.3 | 5.70 | x |
| 114 | 583.4 | 6.21 | x |
| 115 | 607.5 | 5.96 | x |
| 116 | 493.3 | 5.41 | x |
| 117 | 605.4 | 5.94 | x |
| 118 | 655.4 | 4.01 | x |
| 119 | 655.5 | 4.16 | x |
| 120 | 525 | 5.79 | x |
| 121 | 529.5 | 5.59 | x |
| 122 | 553.5 | 5.87 | x |
| 123 | 553.3 | 5.89 | x |
| 124 | 617.4 | 6.21 | x |
| 125 | 523.3 | 5.49 | x |
| 126 | 575.3 | 5.72 | x |
| 127 | 611.4 | 6.2 | x |
| 128 | 585.5 | 6.23 | x |
| 129 | 586.5 | 6.18 | x |
| 130 | 507.3 | 5.73 | x |
| 131 | 519.3 | 5.78 | x |
| 132 | 613.5 | 6.08 | x |
| 133 | 517.4 | 6.51 | x |
| 134 | 585.4 | 6.83 | x |
| 135 | 539.3 | 5.87 | x |
| 136 | 531.4 | 5.89 | x |
| 137 | 607.3 | 6.29 | x |
| 138 | 593.5 | 6.005 | x |
| 139 | 521.5 | 6.10 | x |
| 140 | 519.2 | 5.93 | x |
| 141 | 511.2 | 5.94 | x |
| 142 | 588.4 | 6.08 | x |
| 143 | 562.5 | 4.86 | x |
| 144 | 527.3 | 5.96 | x |
| 145 | 577.2 | 6.31 | x |
| 146 | 435.3 | 5.46 | x |
| 147 | 620.5 | 5.707 | x |
| 148 | 505.1 | 6.46 | x |
| 149 | 485.2 | 6.26 | x |
| 150 | 491.2 | 5.69 | x |
| 151 | 573.1 | 6.07 | x |
| 152 | 565.2 | 6.88 | x |
| 153 | 483.4 | 5.77 | x |
| 154 | 623.2 | 6.41 | x |
| 155 | 607.5 | 6.6 | x |
| 156 | 539.4 | 6.32 | x |
| 157 | 525.3 | 6.03 | x |
| 158 | 601.3 | 6.09 | x |
| 159 | 601.3 | 6.10 | x |
| 160 | 613.4 | 6.31 | x |
| 161 | 645.3 | 6.28 | x |
| 162 | 640.2 | 6.7 | x |
| 163 | 589.4 | 6.24 | x |
| 164 | 559.1 | 5.90 | x |
| 165 | 627.4 | 6.56 | x |
| 166 | 619.2 | 4.41 | x |
| 167 | 606.2 | 5.16 | x |
| 168 | 619.4 | 4.447 | x |
| 169 | 525.3 | 6.13 | x |
| 170 | 491.2 | 5.63 | x |
| 171 | 603.2 | 6.96 | xx |
| 172 | 603.2 | 7.15 | x |
| 173 | 635.3 | 4.75 | x |
| 174 | 635.3 | 4.75 | x |
| 175 | 618.3 | 5.54 | x |
| 176 | 519.3 | 5.744 | x |
| 177 | 567.3 | 6.337 | x |
| 178 | 574.2 | 6.138 | x |
| 179 | 601.3 | 6.692 | x |
| 180 | 591.3 | 7.237 | x |
| 181 | 625.4 | 6.570 | x |
| 182 | 575.4 | 6.404 | x |
| 183 | 575.4 | 6.274 | x |
| 184 | 499.3 | — | x |
| 185 | 589.3 | 6.37 | x |
| 186 | 589.4 | 6.12 | x |

x = <10 μM; xx = <1 μM

### Example 75

### Inhibition or Stimulation of cAMP Signal in Cells Expressing Human MC1R Transient Transfection of Mammalian Cell Lines

The mammalian cell line, human embryonic kidney cells (HEK 293), were maintained in Dulbeccos Modified Eagle's medium (DMEM) with 5% fetal bovine serum, L-glutamine, high glucose and antibiotics/antimycotics. On the day prior to transfection, cells were passaged using trypsin/EDTA and seeded into 75 cm² flasks so that they would be approximately 90% confluent the next day. The next day, the cell media was replaced with fresh antibiotic/antimycotic-containing DMEM. Approximately 100 μl of the transfection lipid Turbofectin 8.0 (Origene Technologies, MD, USA), was diluted in 1.0 mL of serum and antibiotic/antimycotic-free OptiMEM in a sterile 15 mL tube and incubated for 5 mins at room temperature. Following incubation, approximately 10-20 g of plasmid DNA expressing the gene of interest (for example: Homo sapiens melanocortin 1 receptor (Origene Technologies, MD, USA)) was diluted into the transfection mix and incubated for a further 30 mins at room temperature. The DNA/lipid solution was then added drop-wise to the media covering the cells while rocking the flask gently. 24 hrs post-transfection, the cells were passaged and seeded directly into two, 75 cm² flasks and left to recover. 48 hrs post transfection, cells were harvested for use in assays with cell dissociation solution.

Cyclic-Adenosine Monophosphate [cAMP]Stimulation Assay:

HEK 293 cells transiently expressing the melanocortin MC1 receptor were suspended in stimulation buffer (Hanks buffered saline solution (HBSS), 0.1% bovine serum albumin, protease inhibitors and 0.5 mM 3-Isobutyl-1-methylxanthine) at 4×10⁶ cells/mL. 5 μl of cells, plus the compounds/peptides as described below, were added to wells of a 384-well plate as soon as possible after resuspension.

To detect antagonist activity, test compounds at varying concentrations were diluted in stimulation buffer at four times concentrate and 2.5 μl was added to wells containing cells. 2.5 μl of a four times required concentration of NDP-MSH or alpha-MSH was added to all wells containing compounds. Negative control wells contained two times concentrated NDP-MSH or alpha-MSH alone without compound.

To detect agonist activity, test compounds at varying concentrations were diluted in stimulation buffer at two times concentrate and 5 μl was added to wells containing cells. Positive control wells contained NDP-MSH or alpha-MSH alone (no compound) at two times concentrate

Basal level (of cAMP) control wells contained stimulation buffer only (no agonist or compounds). Known concentrations of cAMP (standards) in stimulation buffer were included on the plate, but no cells were added to these wells. The plate was then incubated for 30 mins at 37° C. with gentle shaking. After incubation, 10 μl of lysis buffer (10% Tween 20, 1 M HEPES, 0.1% BSA, protease inhibitors, ddH₂O) was added to all wells to be measured. Detection of cAMP was then achieved using the Alphascreen cAMP kit (Perkin Elmer, USA), briefly described as follows. A dilution of 10 μl acceptor beads/mL of lysis buffer was prepared in low light conditions. 5 μl of diluted acceptor beads were added to each well to be measured, then the plate was incubated for 30 mins at room temperature, in the dark, with gentle shaking. In low light conditions, donor beads were diluted at 10 μl/mL of lysis buffer, to which 0.75 μl biotinylated cAMP/mL of lysis buffer was added. This mixture was allowed to incubate for 30 mins at room temperature (in the dark) before proceeding with the assay. Following incubation, 5 μl/mL of biotinylated cAMP/Donor bead mix were added per well in low light conditions and the plate was incubated in the dark, at room temperature, for a further hr. Plates were read on an Envision plate reader (Perkin Elmer) after 1 hr and ˜16 hrs incubation. cAMP concentration in the cells was determined by the use of a ‘standard curve’ generated from the output of known cAMP concentrations as described below.

Each assay plate contained a “standard curve” of known concentrations of cAMP, in 10 fold dilutions. This is an essential part of the assay as there is high inter-plate variability. The plates were read on an Envision multilabel plate reader fitted with Alphascreen technology and the raw data was imported into GraphPad Prism 4 software (GraphPad, USA) for analysis. A curve was fitted to the known concentrations using non-linear regression, specifically using a sigmoidal dose-response equation (Y=Bottom+(Bottom+(Top-Bottom)/1+10^{logEC50-X}), where the equation shows the response as a function of the logarithm of concentration. X is the logarithm of peptide/compound concentration and Y is the response. Also considered in this equation are bottom plateau, top plateau of the curve and EC₅₀ (effective concentration, 50%)

In a similar assay testing for MC1R agonism, MM96L cells were transiently transfected with wild type MC1R and stimulated with compound (10 μM) for different time points, with cAMP accumulation compared to basal cAMP levels and the cAMP response to stimulation with NDP-MSH

In yet another assay HEK293 cells stably expressing MC1R were incubated with compound (100 nM to 100 μM) for 30 min, then lysed and measured by Western blotting using an antibody specific to the phosphorylated form of CREB (cAMP responsive element binding protein), which is activated by cAMP and hence is a surrogate measure of cAMP activation by MC1R

### Example 76

### Activity of Selected Compounds: hMC1R Agonism

Representative compounds of the present invention were tested for agonism of the hMC1R, as in Example 75, results are listed in Table 5.

TABLE 5
Assay of hMC1 Agonism by Selected Compounds

| | | human MC1R agonism | human MC1R agonism |
|---|---|---|---|
| | | (cAMP, fold incease | (increase in |
| | human MC1R | over basal level, | phosphoCREB, 30 min |
| | agonism | 30 min stimulation | stimulation with |
| | (cAMP EC₅₀ | with 10 μM | 0.1-100 μM |
| | HEK293 cell) | compound, | compopund), |
| Cpd. | (μM) | MM96L cells) | HEK293 cell) |
| 110 | 78 | 1.5 | not assayed |
| 112 | 11 | not assayed | not assayed |
| 116 | 16 | not assayed | not assayed |
| 119 | 7 | not assayed | not assayed |
| 136 | not assayed | 1.5 | not assayed |
| 184 | not assayed | not assayed | + |
| 185 | not assayed | 1.5 | not assayed |
| 186 | not assayed | 2.5 | not assayed |

### REFERENCES

Abdel-Malek, Z. A.; Kadekaro, A. L.; Kavanagh, R. J.; Todorovic, A.; Koikov, L. N.; McNulty, J. C.; Jackson, P. J.; Millhauser, G. L.; Schwemberger, S.; Babcock, G.; Haskell-Luevano, C.; Knittel, J. J. FASEB J. 2006, 20, E888-E896 “Melanoma prevention strategy based on using tetrapeptide α-MSH analogs that protect human melanocytes from UV-induced DNA damage and cytotoxicity”
Abdel-Malek, Z. A.; Knittel, J. J.; Kadekaro, A. L.; Swope, V. B.; Starner, R. Photochem. Photobiol. 2008, 84, 501-508 “The Melanocortin-1 Receptor and the UV Response of Human Melanocytes—A Shift in Paradigm”
Bagutti, C.; Stolz, B.; Albert, R.; Bruns, C.; Pless, J.; Eberle, A. N. Int. J. Cancer 1994, 58, 749-755“[111In]-DTPA-labeled analogues of alpha-melanocyte-stimulating hormone for melanoma targeting: receptor binding in vitro and in vivo”.
Bard, D. R. Nucl. Med. Commun. 1995, 16, 860-866 “An improved imaging agent for malignant melanoma, based on [Nle4,D-Phe7]alpha-melanocyte stimulating hormone.”
Barnetson, R. S.; Ooi, T. K. T.; Zhuang, L.; Halliday, G. M.; Reid, C. M.; Walker, P. C.; Humphrey, S. M.; Kleinig, M. J. J. Invest. Dermatol. 2006, 126, 1869-1878“[Nle⁴-D-Phe⁷]-α-Melanocyte-Stimulating Hormine Significantly Increased Pigmentation and Decreased UV Damage in Fair-Skinned Caucasion Volunteers”
Bednarek, M. A.; MacNeil, T.; Tang, R.; Fong, T. M.; Cabello, M. A.; Maroto, M.; Teran, A. peptides 2008, 29, 1010-1017 “Cyclic analogs of α-melanocyte-stimulating hormone (αMSH) with high agonist potency and selectivity at human melanocortin receptor 1b”
Berardesca, E.; Adrigo, M.; Berardesca, M.; Camelli, N. Expert Rev. Dermatol. 2008, 3, 187-193 “Melasma: current and future treatments”
Bohm, M.; Luger, T. A.; Tobin, D. J.; Garcia-Borron, J. C. J. Invest. Dermatol. 2006, 126, 1966-1975 “Melanocortin Receptor Ligands: New Horizons for Skin Biology and Clinical Dermatology”
Bonetto, S.; Carlavan, I.; Baty, D. Peptides 2005, 26, 2302-2313 “Isolation and characterization of antagonist and agonist peptides to the human melanocortin 1 receptor”
Box, N. F.; Duffy, D. L.; Irving, R. E.; Russell, A.; Chen, W.; Griffiths, L. R.; Parsons, P. G.; Green, A. C.; Sturm, R. A J. Invest. Dermatol. 2001, 116, 224-229 “Melanocortin-1 Receptor Genotype is a Risk Factor for Basal and Squamous Cell Carcinoma”
Brown, D. A. J Photochem. Photobiol. B 2001, 63, 101-110 “Skin pigmentation enhancers”
Candille, S. I.; Kaelin, C. B.; Cattanach, B. M.; Yu, B.; Thompson, D. A.; Nix, M. A.; Kerns, J. A.; Schmutz, S. M.; Milhauser, G. L.; Barsh, G. S. Science 2007, 318, 1418-1423 “A β-Defensin Mutation Causes Black Coat Color in Domestic Dogs”
Catania, A.; Gatti, S.; Colombo, G.; Lipton, J. M. Pharmacol. Rev. 2004, 56, 1-29 “Targeting Melanocortin Receptors as a Novel Strategy to Control Inflammation”
Chen, J.; Giblin, M. F.; Wang, N.; Jurisson, S. S.; Quinn, T. P. Nucl. Med. Biol. 1999, 26, 687-693 “In vivo evaluation of ⁹⁹mTc/¹⁸⁸Re-labeled linear alpha-melanocyte stimulating hormone analogs for specific melanoma targeting”
Chen, J.; Cheng, Z.; Hoffman, T. J.; Jurisson, S. S.; Quinn, T. P. Cancer Res. 2000, 60, 5649-5658 “Melanoma-targeting properties of ⁹⁹mtechnetium-labeled cyclic alpha-melanocyte-stimulating hormone peptide analogues”
Chen, J.; Cheng, Z.; Owen, N. K. et al. J. Nucl. Med. 2001, 42, 1847-1855 “Evaluation of an ¹¹¹In-DOTA-rhenium cyclized alpha-MSH analog: a novel cyclic-peptide analog with improved tumor targeting properties”
Cheng, Z.; Chen, J.; Miao, Y.; Owen, N. K.; Quinn, T. P.; Jurisson, S. S. J. Med. Chem. 2002, 45, 3048-3056 “Modification of the structure of a metallopeptide: synthesis and biological evaluation of ¹¹¹In-labeled DOTA-conjugated rhenium-cyclized alpha-MSH analogues”
Cheng, Z.; Chen, J.; Quinn, T. P.; Jurisson, S. S. Cancer Res. 2004, 64, 1411-1418 “Radioiodination of rhenium cyclized alpha-melanocyte-stimulating hormone resulting in enhanced radioactivity localization and retention in melanoma”
Cheng, Z.; Xiong, Z. M.; Wu, Y.; Zhang, X.; Chen, X.; Gambhir, S. S. Mol. Imaging. Biol. 2005, 7, 126 “MicroPET imaging of melanoma using Cu-64 labeled alpha-melanocyte stimulating hormone peptide analogue”
Cheng, Z.; Xiong, Z. M.; Subbarayan, M.; Chen, X.; Gambhir, S. S.; Bioconjug. Chem. 2007, 18, 765-772“⁶⁴Cu labeled alphamelanocyte stimulating hormone analog for microPET imaging of melanocortin 1 receptor expression”
Chhajlani, V; Wikberg, J. E. S. FEBS Lett., 2002, 309, 417-420 “Molecular cloning and expression of the human melanocyte stimulating hormone receptor cDNA”
De-Misa, R. F.; Hernández-Jimenez, J. G.; Hernández, G. C.; Pérez-Méndez, L.; Aguirre-Jaime, A.; Flores, C. Hernandez, J. S.; Molinero, A. P.; Clayeríe-Martin, F. J. Dermatol. Sc. 2008, 52, 186-192 “The D84E variant of the α-MSH receptor 1 gene is associated with cutaneous maloignant melanoma early onset”
Dorr, R. T.; Dvorakova, K.; Brooks, C.; Lines, R.; Levine, N.; Schram, K.; Miketova, P.; Hruby, V.; Alberts, D. S Photochem. Photobiol. 2000, 72, 526-532 “Increased Eumelanin Expression and Tanning is Induced by a Superpotent Melanotropin [Nle⁴-D-Phe⁷]-α-MSH in Humans”
Dorr, R. T.; Ertl, G.; Levine, N.; Brooks, C.; Bangert, J. L.; Powell, M. B.; Humphrey, S.; Alberts, D. S Arch. Dermatol. 2004, 140, 827-835 “Effects of a Superpotent Melanotropic Peptide in Combination with Solar UV Radiation on Tanning of the Skin in Human Volunteers”
FitzGerald, L. M.; Fryer, J. L.; Dwyer, T.; Humphrey, S. M. Peptides 2006, 27, 388-394 “Effect of MELANOTAN®, [Nle⁴-D-Phe⁷]-α-MSH], on melanin synthesis in humans with MC1R variant alleles”
Froidevaux, S.; Calame-Christe, M.; Tanner, H.; Sumanovski, L.; Eberle, A. N. J. Nucl. Med. 2002, 43, 1699-1706 “A novel DOTA-alpha-melanocyte-stimulating hormone analog for metastatic melanoma diagnosis”
Froidevaux, S.; Calame-Christe, M.; Schuhmacher, J. et al. J. Nucl. Med. 2004, 45, 116-123 “A gallium-labeled DOTA-alpha-melanocyte-stimulating hormone analog for PET imaging of melanoma metastases”
Froidevaux, S.; Calame-Christe, M.; Tanner, H.; Eberle, A. N. J. Nucl. Med. 2005, 46, 887-895 “Melanoma targeting with DOTA-alpha-melanocyte-stimulating hormone analogs: structural parameters affecting tumor uptake and kidney uptake”
García-Barrón, J. C.; Sánchez-Laorden, B. L.; Jimenez-Cervantes, C. Pigment Cell Res. 2005, 18 393-410 “Melanocortin-1 receptor structure and functional regulation”
Gawkrodger, D. J.; Ormerod, A. D.; Shaw, L.; Mauri-Sole, I.; Whitton, M. E.; Watts, M. J.; Anstey, A. V.; Ingham, J.; Young, K. Brit. J. Dermatol. 2008, 159, 1051-1076 “Guideline for the diagnosis and management of vitiligo”
Getting, S. J. Trends Pharmacol. Sci. 2002, 23, 447-449 “Melanocortin peptides and their receptors: new targets for anti-inflammatory therapy”
Hadley, M. E.; Dorr, R. T. Peptides 2006, 27, 921-930 “Melanocortin peptide therapeutics: Historical milestones, clinical studies and commercialization”
Halder, R. M.; Nootheri, P. K. J. Amer. Acad. Dermatol. 2003, 48(suppl), S143-S148 “Ethnic skin disorders overview”
Haskell-Luevano, C.; Rosenquist, A.; Souers, A.; Khong, K. C.; Ellman, J. A.; Cone, R. D. J. Med. Chem. 1999, 42, 4380-4387 “Compounds That Activate the Mouse Melanocortin-1 Receptor Identified by Screening a Small Molecule Library Based upon the β-Turn”
Holder, J. R.; Bauzo, R. M.; Xiang, Z.; Haskell-Luevano, C. J. Med. Chem. 2002, 45, 3073-3081 “Structure-Activity Relationships of the Melanocortin Tetrapeptide Ac-His-DPhe-Arg-Trp-NH₂ at the Mouse Melanocortin Receptors: Part 2 Modifications at the Phe Position”
Holder, J. R.; Marques, F. F.; Xiang, Z.; Bauzo, R. M.; Haskell-Luevano, C. Eur. J. Pharmacol. 2003, 462, 41-52 “Characterization of apliphatic, cyclic and aromatic N-terminally “capped” His-DPhe-Arg-Trp-NH₂ tetrapeptides at the melanocortin receptors”
Joseph, C. G.; Wilson, K. R.; Wood, M. S.; Sorenson, N. B.; Phan, D. V.; Xiang, Z.; Witek, R. M.; Haskell-Luevano, C. J. Med. Chem. 2008, 51, 1423-1431 “The 1,4-Benzodiazepine-2,5-dione Small Molecule Template Results in Melanocortin Receptor Agonists with Nanomolar Potencies”
Kingo, K.; Aunin, E.; Karelson, M.; Philips, M.-A.; Rätsep, R.; Silm, H.; Vasar, E.; Soomets, U.; Kõks, S.; J. Dermatol. Sci. 2007, 48, 113-122 “Gene expression analysis of melanocortin system in vitiligo”
Levine, N.; Sheftel, S. N.; Eytan, T.; Dorr, R. T.; Hadley, M. E.; Weinrach, J. C.;. Ertl, G. A; Toth, K.; McGee, D. L.; Hruby, V. J. JAMA, 1991, 266, 2730-2736 “Induction of skin tanning by subcutaneous administration of a potent synthetic melanotropin”
Lin, J. Y.; Fisher, D. E. Nature 2007, 445, 843-850 “Melanocyte biology and skin pigmentation”
Mazur, A. W.; Kulesza, A.; Mishra, R. K.; Cross-Doersen, D.; Russell, A. F.; Ebetino, F. H. Bioorg. Med. Chem. 2003, 11, 3053-3063 “Novel tetrahydropyran-based peptidomimetics from a bioisosteric transformation of a tripeptide. Evidence of their activity at melanocortin receptors”
McQuade, P.; Miao, Y. Yoo, J.; Quinn, T. P.; Welch, M. J.; Lewis, J. S. J. Med. Chem. 2005, 48, 2985-2992 “Imaging of melanoma using 64Cu- and 86Y-DOTA-ReCCMSH(Arg11), a cyclized peptide analogue of alpha-MSH”
Miao, Y.; Owen, N. K.; Fisher, D. R.; Hoffman, T. J.; Quinn, T. P. J. Nucl. Med. 2005a, 46, 121-129 “Therapeutic efficacy of a 188Re-labeled alpha-melanocyte-stimulating hormone peptide analog in murine and human melanoma-bearing mouse models”
Miao, Y.; Hylarides, M.; Fisher, D. R., et al. Clin. Cancer Res. 2005b, 11, 5616-5621 “Melanoma therapy via peptide-targeted α-radiation”
Miller, K. A.; Gunn, T. M.; Carrasquillo, M. M.; Lamoreux, M. L.; Galbraith, D. B.; Barsh, G. S. Genetics 1997, 146, 1407-1415 “Genetic Studies of the Mouse Mutations mahogany and mahoganoid”
Mogil, J. S.; Wilson, S. G.; Chesler, E. J.; Rankin, A. L.; Nemmani, K. V. S.; Lariviere, W. R.; Groce, M. K.; Wallace, M. R.; Kaplan, L.; Staud, R.; Ness, T. J.; Glover, T. L.; Stankova, M.; Mayorov, A.; Hruby, V. J.; Grisel, J. E.; Fillingim, R. B. PNAS 2003, 100, 4867-4872 “The melanocortin-1 receptor gene mediates female-specific mechanisms of analgesia in mice and humans”
Mogil, J. S.; Ritchie, J.; Smith, S. B.; Strasburg, K.; Kaplan, L.; Wallace, M. R.; Romberg, R. R.; Bijl, H.; Sarton, E. Y.; Fillingim, R. B.; Dahan, A. J. Med. Genet. 2005, 42, 583-587 “Melanocortin-1 receptor gene variants affect pain and μ-opioid analgesia in mice and humans”
Mountjoy, K. G.; Robbins, L. S.; Mortrud, M. T.; Cone, R. D. Science 1992, 257 1248-1251 “The cloning of a family of genes that encode the melanocortin receptors”
Mutulis, F.; Mutule, I.; Liepinsh, E.; Yahorau, A.; Lapinsh, M.; Kopantshuk, S.; Veiksina, S.; Rinken, A.; Wikberg, J. E. S. Peptides 2005, 26, 1997-2016 “N-Alkylated dipeptide amides and related structures as imitations of the melanocortins' active core”
Mutulis, F.; Kreieberga, J.; Yahorava, S.; Mutule, I.; Borisova-Jan, L.; Yahorau, A.; Muceniece, R.; Azena, S.; Veiksina, S.; Petrovska, R.; Wikberg, J. E. S. Bioorg. Med. Chem. 2007, 15, 5787-5810 “Design and synthesis of a library of tertiary amide: Evaluation as mimetics of the melanocortins' active core”
Naysmith, L.; Waterston, K.; Ha, T.; Flanagan, N.; Bisset, Y.; Ray, A.; Wakamatsu, K.; Ito, S.; Rees, J. L. J. Invest. Dermatol. 2004, 122, 423-428 “Quantitative Measures of the Effect of the Melanocortin 1 Receptor on Human Pigmentary Status”
Newton, R. A.; Roberts, D. W.; Leonard, J. H.; Sturm, R. A. Peptides 2007, 28, 2387-2396 “Human melanocytes expressing MC1R variant alleles show imparied activation of multiple signaling pathways”
Olumide, Y. M.; Akinkugbe, A. O.; Altraide, D.; Mohammed, T.; Ahamefule, N.; Ayanlowo, S.; Onyekonwu, C.; Essen, N. Int. J. Dermatol. 2008, 47, 344-353 “Complications of chronic use of skin lightenting cosmetics”
Pharoah, P. D. Nature Genetics 2008, 40, 817-818 “Shedding light on skin cancer”
Quillan, J. M.; Jayawickreme, C. K.; Lerner, M. R. Proc. Natl. Acad. Sci. USA 1995, 92, 2894-2898 “Combinatorial diffusion assay used to identify topically active melanocyte-stimulating hormone receptor antagonists”
Rees, J. L. Pigment Cell Res. 2000, 13, 135-140 “The Melanocortin 1 Receptor (MC1R): More than Just Red Hair”
Rendon, M. I.; Gaviria, J. I. Dermatol. Surg. 2005, 31, 886-889 “Review of Skin-Lightening Agents”
Robbins, L. S.; Nadeau, J. H.; Johnson, K. R.; Kelly, M. A.; Roselli-Rehfuss, L.; Baack, E.; Mountjoy, K. G.; Cone, R. D. Cell 1993, 72, 827-834 “Pigmentation phenotypes of variant extension locus alleles result from point mutations that alter MSH receptor function”
Roberts, D. W.; Newton, R. A; Beaumont, K. A.; Sturm, R. A. Pigment Cell Res. 2006, 19, 76-89 “Quantitative analysis of MC1R gene expression in human skin cell cultures”
Roberts, D. W. J. Invest. Dermatol. 2007, 127, 2472-2473 “MC1R Expression in Skin: Is it Confined to Melanocytes?”
Schaffer, J. V.; Bolognia, J. L. in: Principles of Molecular Medicine. Second Edition. Humana Press 2006 “Disorders of Hypopigmentation”
Siegrist, W.; Solca, F.; Stutz, S. et al. Cancer Res. 1989, 49, 6352-6358 “Characterization of receptors for alphamelanocyte-stimulating hormone on human melanoma cells”
Siegrist. W.; Stutz, S.; Eberle, A. N. Cancer Res. 1994, 54, 2604-2610 “Homologous and heterologous regulation of alpha-melanocyte-stimulating hormone receptors in human and mouse melanoma cell lines”
Slominski, A.; Tobin, D. J.; Shibahara, S.; Wortsman, J. Physiol. Rev. 2004, 84, 1155-1228 “Melanin Pigmentation in Mammalian Skin and Its Hormonal Regulation”
Stratigos, A. I.; Dismisianos, G.; Nikolaou, V.; Poulou, M.; Sypsa, V.; Stefanaki, I.; Papadopoulos, O.; Polydorou, D.; Plaka, M.; Christofidou, E.; Gofas, H.; Tsoutsos, D.; Kastana, O.; Antoniou, C.; Hatzakis, A.; Kanavakis, E.; Katsambas, A. D. J. Invest. Dermatol 2006, 126, 1842-1849 “Melanocortin Receptor-1 Gene Polymorphisms and the Risk of Cutaneous Melanoma in a Low-Risk Southern European Population”
Stulberg, D. L.; Clark, N.; Tovey, D. Am. Fam. Physician 2003a, 68, 1955-1960 “Common Hyperpigmentation Disorders in Adults: Part I. Diagnostic Approach, Café au Lait Macules, Diffuse Hyperpigmentation, Sun Exposure, and Phototoxic Reactions”
Stulberg, D. L.; Clark, N.; Tovey, D. Am. Fam. Physician 2003b, 68, 1963-1968 “Common Hyperpigmentation Disorders in Adults: Part II. Melanoma, Seborrheic Keratoses, Acanthosis Nigricans, Melasma, Diabetic Dermopathy, Tinea Versicolor, and Postinflammatory Hyperpigmentation”
Vaidyanathan, G.; Zalutsky, M. R. Nucl. Med. Biol. 1997, 24, 171-178 “Fluorine-18-labeled [Nle4,D-Phe7]-alpha-MSH, an alpha-melanocyte stimulating hormone analogue”
Verdié, P.; Subra, G.; Feliu, L.; Sanchez, P.; Berge, G.; Garcin, G.; martinez, J. J. Comb. Chem. 2007, 9, 254-262 “On-line synthesis of pseudopeptide library incorporating a benzodiazepine turn mimic: biological evaluation on MC1 receptors”
Voisey, J.; Carroll, L.; van Daal, A. Curr. Drug Targets 2003, 4, 586-597 “Melanocortins and their Receptors and Antagonists”
Wikberg, J. E. S.; Muceniece, R.; Mandrika, I.; Prusis, P.; Lindblom, J.; Post, C.; Skottner, A. Pharmacol. Res. 2000, 42, 393-420 “New Aspects on the Melanocortins and their Receptors”
Wikberg, J. E. S. Exp. Opin. Ther. Patents 2001, 11, 61-76 “Melanocortin receptors: new opportunities in drug discovery”
Wikelgren, I. Science, 2007, 315, 1214-1216 “A Healthy Tan?”

The details of specific embodiments described in this invention are not to be construed as limitations. Various equivalents and modifications may be made without departing from the essence and scope of this invention, and it is understood that such equivalent embodiments are part of this invention.

1. A method of modulating the activity of MC1R or a fragment, analogue or functional equivalent thereof comprising exposing the MC1R or a fragment or analogue or functional equivalent thereof to a compound of the formula (I): 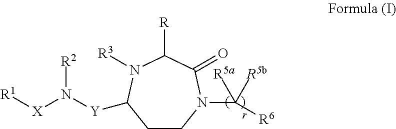
wherein
Y is a group of formula —(CR⁹R¹⁰)ₙ—;
X is selected from the group consisting —C(═O)—, —OC(═O)—, —NHC(═O)—, —(CR¹¹R¹²)ₛ, and —S(═O)₂—;
R is an amino acid side chain group;
R¹ is selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂heterocycloalkyl, optionally substituted C₆-C₁₈aryl, and optionally substituted C₁-C₁₈heteroaryl;
R² and R³ are each independently selected from the group consisting of H, optionally substituted C₁-C₁₂alkyl, optionally substituted C₂-C₁₂alkenyl, optionally substituted C₂-C₁₂alkynyl, optionally substituted C₁-C₁₂heteroalkyl, optionally substituted C₃-C₁₂cycloalkyl, optionally substituted C₂-C₁₂heterocycloalkyl, optionally substituted C₆-C₁₈aryl, and optionally substituted C₁-C₁₈heteroaryl, or
R² and R³ may be joined to form a linker between the two nitrogen atoms to which they are attached, wherein the linker is selected from the group consisting of —C(═O)—, —CH₂—, —C(═O)CH₂— and —CH₂C(═O)—;
R^{5a}, R^{5b} and R⁶ are each independently selected from the group consisting of H, halogen, hydroxy, optionally substituted C₁-C₁₂alkyl, optionally substituted C₂-C₁₂alkenyl, optionally substituted C₂-Ciz₂alkynyl, optionally substituted C₁-C₁₂ heteroalkyl, optionally substituted C₁-C₁₀ heteroalkenyl, optionally substituted C₃-C₁₂cycloalkyl, optionally substituted C₂-C₁₂ heterocycloalkyl, optionally substituted C₆-C₁₈aryl, optionally substituted C₁-C₁₈heteroaryl, optionally substituted amino, optionally substituted carboxy, optionally substituted C₁-C₁₂alkyloxy, and optionally substituted thio;
each R⁹ and R¹⁰ is independently selected from the group consisting of H, optionally substituted C₁-C₁₂alkyl, optionally substituted C₆-C₁₈aryl, and optionally substituted C₁-C₁₈heteroaryl;
each R¹¹ and R¹² is independently selected from the group consisting of H, and optionally substituted C₁-C₁₂alkyl;
n is an integer selected from the group consisting of 1, 2, 3 and 4;
r is an integer selected from the group consisting of 0, 1, 2, 3, and 4;
s is an integer selected from the group consisting of 0, 1, 2, 3, and 4;
or a pharmaceutically acceptable salt or prodrug thereof.

2. A method of preventing, treating, or inhibiting a condition in a mammal, wherein the condition is selected from the group consisting of (i) a condition associated with the activity or presence of MC1R or a fragment, analogue or functional equivalent thereof in a mammal and (ii) a condition that may be prevented or treated by modification of skin pigmentation in the mammal, the method comprising administering a therapeutically effective amount of a compound of formula (I) as described in claim 1 to the mammal.

3. A composition for inducing UV-independent pigmentation of human skin and/or for enhancing UV-dependent pigmentation of human skin, comprising a compound of formula (I) as described in claim 1 and a dermatologically acceptable carrier, excipient or diluent, wherein the composition is formulated to penetrate the human skin to the stratum basale.
